SEQUENCE LISTING (1 sequence)

SEQ ID NO: 1 - DNA, 40 bp, wild-type homo sapiens
taaattggatccaccatggaatcatctttctcatttggag

(19) United States
(12) Patent Application Publication
(10) Pub. No.: US 2011/0020460 A1
(43) Pub. Date: Jan. 27, 2011

(54) GPR 119 MODULATORS

(75) Inventors: Vincent Mascitti, Groton, CT (US); Kim F. McClure, Mystic, CT (US); Michael J. Munchhof, Salem, CT (US); Ralph P. Robinson, Gales Ferry, CT (US)

Correspondence Address: PFIZER INC.;PATENT DEPARTMENT, Bld 114 M/S 9114, EASTERN POINT ROAD, GROTON, CT, 06340, US

(73) Assignee: Pfizer Inc.

(21) Appl. No.: 12/793,938

(22) Filed: Jun. 4, 2010

Related U.S. Application Data
(60) Provisional application No. 61/184,355, filed on Jun. 5, 2009.
(60) Provisional application No. 61/257,621, filed on Nov. 3, 2009.

(51) Int. Cl. A61K 31/454; C07D 401/04; C07D 401/14; C07F 9/40; A61K 31/4545; A61K 31/675; A61K 31/55; A61K 31/536; A61K 31/485; A61K 31/566; A61K 38/22; A61K 31/46; A61K 31/473; A61K 31/4965; A61K 31/472; A61K 31/702; A61K 38/16; A61K 35/56; A61K 31/4985; A61P 3/10; A61P 3/04; A61P 3/00; A61P 3/06; A61P 9/10; A61P 1/00; A61P 1/04

(52) U.S. Cl. 424/520; 546/211; 546/193; 546/22; 546/194; 514/326; 514/318; 514/89; 514/217.01; 514/230.5; 514/282; 514/171; 514/5.3; 514/299; 514/284; 514/255.06; 514/309; 514/217.11; 514/61; 514/6.9; 514/249

(57) ABSTRACT

Compounds of Formula I that modulate the activity of the G-protein-coupled receptor GPR119 and their uses in the treatment of diseases linked to the modulation of the G-protein-coupled receptor GPR119 in animals are described herein.

### FIELD OF THE INVENTION

The present invention relates to a new class of cyanopyrazoles, pharmaceutical compositions containing these compounds, and their use to modulate the activity of the G-protein-coupled receptor, GPR119.

### BACKGROUND

Diabetes mellitus are disorders in which high levels of blood glucose occur as a consequence of abnormal glucose homeostasis. The most common forms of diabetes mellitus are Type I (also referred to as insulin-dependent diabetes mellitus) and Type II diabetes (also referred to as non-insulin-dependent diabetes mellitus). Type II diabetes, accounting for roughly 90% of all diabetic cases, is a serious progressive disease that results in microvascular complications (including retinopathy, neuropathy and nephropathy) as well as macrovascular complications (including accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin, thiazolidinediones, or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion only in the presence of high glucose, but must be injected due to a lack of oral bioavailability. Sitagliptin, a dipeptidyl peptidase IV inhibitor, is a new drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

In Type II diabetes, muscle, fat and liver cells fail to respond normally to insulin. This condition (insulin resistance) may be due to reduced numbers of cellular insulin receptors, disruption of cellular signaling pathways, or both. At first, the beta cells compensate for insulin resistance by increasing insulin output. Eventually, however, the beta cells become unable to produce sufficient insulin to maintain normal glucose levels (euglycemia), indicating progression to Type II diabetes.

In Type II diabetes, fasting hyperglycemia occurs due to insulin resistance combined with beta cell dysfunction. There are two aspects of beta cell defect dysfunction: 1) increased basal insulin release (occurring at low, non-stimulatory glucose concentrations). This is observed in obese, insulin-resistant pre-diabetic stages as well as in Type II diabetes, and 2) in response to a hyperglycemic challenge, a failure to increase insulin release above the already elevated basal level. This does not occur in pre-diabetic stages and may signal the transition from normo-glycemic insulin-resistant states to frank Type II diabetes. Current therapies to treat the latter aspect include inhibitors of the beta-cell ATP-sensitive potassium channel to trigger the release of endogenous insulin stores, and administration of exogenous insulin. Neither achieves accurate normalization of blood glucose levels and both carry the risk of eliciting hypoglycemia.

Thus, there has been great interest in the discovery of agents that function in a glucose-dependent manner. Physiological signaling pathways which function in this way are well known, including gut peptides GLP-1 and GIP. These hormones signal via cognate G-protein coupled receptors to stimulate production of cAMP in pancreatic beta-cells. Increased cAMP apparently does not result in stimulation of insulin release during the fasting or pre-prandial state. However, a number of biochemical targets of cAMP, including the ATP-sensitive potassium channel, voltage-sensitive potassium channels and the exocytotic machinery, are modulated such that insulin secretion due to postprandial glucose stimulation is significantly enhanced. Therefore, agonist modulators of novel, similarly functioning, beta-cell GPCRs, including GPR119, would also stimulate the release of endogenous insulin and promote normalization of glucose levels in Type II diabetes patients. It has also been shown that increased cAMP, for example as a result of GLP-1 stimulation, promotes beta-cell proliferation, inhibits beta-cell death and thus improves islet mass. This positive effect on beta-cell mass should be beneficial in Type II diabetes where insufficient insulin is produced.

It is well known that metabolic diseases have negative effects on other physiological systems and there is often co-occurrence of multiple disease states (e.g. Type I diabetes, Type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy. Thus, treatment of the diabetic condition should be of benefit to such interconnected disease states.

### SUMMARY OF THE INVENTION

In accordance with the present invention, a new class of GPR 119 modulators has been discovered. These compounds may be represented by Formula I, as shown below:

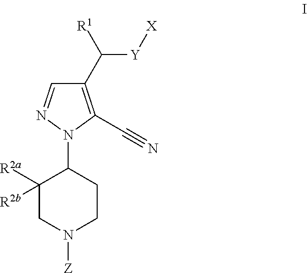

wherein:

X is

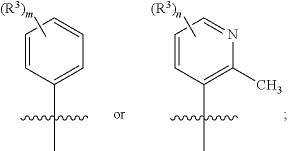

Y is O, CH(R⁵), or NR⁵;

Z is —C(O)—O—R⁶ or pyrimidine substituted with C₁-C₄ alkyl, CF₃, halogen, cyano, C₃-C₆ cycloalkyl or C₃-C₆ cycloalkyl wherein one carbon atom of said cycloalkyl moiety may optionally be substituted with methyl or ethyl;

m is 1, 2, or 3;

n is 0, 1 or 2;

R¹ is hydrogen, C₁-C₄ alkyl, or C₃-C₆ cycloalkyl;

R^{2a} is hydrogen, fluoro or C₁-C₄ alkyl;
R^{2b} is hydrogen or fluoro, with the proviso that when R^{2a} is C₁-C₄ alkyl, R^{2b} is hydrogen;

each R³ is individually selected from the group consisting of: hydroxy, halogen, cyano, C₁-C₄ alkyl, C₁-C₄ alkoxy, C₁-C₄ haloalkyl, C₁-C₄ haloalkoxy, —SO₂—R⁷, —P(O)(OR⁸)(OR⁹), —C(O)—NR⁸R⁹, —N(CH₃)—CO—O—(C₁-C₄)alkyl, —NH—CO—O—(C₁-C₄)alkyl, —NH—CO—(C₁-C₄)alkyl, —N(CH₃)—CO—(C₁-C₄)alkyl, —NH—(CH₂)₂—OH and a 5 to 6-membered heteroaryl group containing 1, 2, 3 or 4 heteroatoms each independently selected from oxygen, nitrogen and sulfur, wherein a carbon atom on said heteroaryl group is optionally substituted with R^{4a} or a nitrogen atom on said heteroaryl group is optionally substituted with R^{4b};

R^{4a} is hydrogen, C₁-C₄ alkyl, C₁-C₄ alkoxy, C₁-C₄ haloalkyl, or halogen, wherein said alkyl is optionally substituted with hydroxyl or C₁-C₄ alkoxy;

R^{4b} is hydrogen, C₁-C₄ alkyl, —CH₂-C₁-C₃ haloalkyl, -C₂-C₄ alkyl-OH or —CH₂-C₁-C₄ alkoxy;

R⁵ is hydrogen or when R¹ is hydrogen then R⁵ is hydrogen or C₁-C₄ alkyl;

R⁶ is C₁-C₄ alkyl or C₃-C₆ cycloalkyl wherein one carbon atom of said cycloalkyl moiety may optionally be substituted with methyl or ethyl;

R⁷ is represented by C₁-C₄ alkyl, C₃-C₆ cycloalkyl, NH₂, or —(CH₂)₂—OH;

R⁸ is represented by hydrogen or C₁-C₄ alkyl; and

R⁹ is represented by hydrogen, C₁-C₄ alkyl, C₃-C₆ cycloalkyl, —(CH₂)₂—OH, —(CH₂)₂—O—CH₃, —(CH₂)₃—OH, —(CH₂)₃—O—CH₃, 3-oxetanyl, or 3-hydroxycyclobutyl;

or when R³ is —C(O)—NR⁸R⁹, R⁸ and R⁹ can be taken together with the nitrogen atom to which they are attached to form an azetidine, pyrrolidine, piperidine or morpholine ring;

or a pharmaceutically acceptable salt thereof.

Moreover, the present invention is directed at the compounds:

1-methylcyclopropyl 4-{4-[(4-carbamoyl-3-fluorophenoxy)methyl]-5-cyano-1H-pyrazol-1-yl}piperidine-1-carboxylate;

1-methylcyclopropyl 4-{4-[(4-carbamoyl-2-fluorophenoxy)methyl]-5-cyano-1H-pyrazol-1-yl}piperidine-1-carboxylate;

isopropyl 4-(5-cyano-4-{[4-(1H-pyrazol-1-yl)phenoxy]methyl}-1H-pyrazol-1-yl)piperidine-1-carboxylate;

1-methylcyclopropyl 4-{5-cyano-4-[(2,3-difluorophenoxy)methyl]-1H-pyrazol-1-yl}piperidine-1-carboxylate;

1-methylcyclopropyl 4-{5-cyano-4-[(2,5-difluorophenoxy)methyl]-1H-pyrazol-1-yl}piperidine-1-carboxylate;

1-methylcyclopropyl 4-{5-cyano-4-[(2,3,6-trifluorophenoxy)methyl]-1H-pyrazol-1-yl}piperidine-1-carboxylate;

isopropyl 4-[5-cyano-4-({2-fluoro-4-[1-(2-hydroxyethyl)-1H-tetrazol-5-yl]phenoxy}methyl)-1H-pyrazol-1-yl]piperidine-1-carboxylate;

isopropyl 4-[5-cyano-4-({2-fluoro-4-[2-(2-hydroxyethyl)-2H-tetrazol-5-yl]phenoxy}methyl)-1H-pyrazol-1-yl]piperidine-1-carboxylate;

isopropyl 4-(5-cyano-4-{[2-fluoro-4-(1-methyl-1H-imidazol-2-yl)phenoxy]methyl}-1H-pyrazol-1-yl)piperidine-1-carboxylate;

1-methylcyclopropyl 4-{5-cyano-4-[(4-cyanophenoxy)methyl]-1H-pyrazol-1-yl}piperidine-1-carboxylate;

1-methylcyclopropyl 4-{4-[(4-carbamoylphenoxy)methyl]-5-cyano-1H-pyrazol-1-yl}piperidine-1-carboxylate;

1-methylcyclopropyl 4-(5-cyano-4-{[4-(1-methyl-1H-tetrazol-5-yl)phenoxy]methyl}-1H-pyrazol-1-yl)piperidine-1-carboxylate;

1-methylcyclopropyl 4-(5-cyano-4-{[2-fluoro-4-(1-methyl-1H-tetrazol-5-yl)phenoxy]methyl}-1H-pyrazol-1-yl)piperidine-1-carboxylate;

isopropyl 4-(5-cyano-4-{[2-fluoro-4-(1-methyl-1H-imidazol-5-yl)phenoxy]methyl}-1H-pyrazol-1-yl)piperidine-1-carboxylate;

isopropyl 4-{5-cyano-4-[(2,3,6-trifluorophenoxy)methyl]-1H-pyrazol-1-yl}piperidine-1-carboxylate;

isopropyl 4-{5-cyano-4-[(2,4-difluorophenoxy)methyl]-1H-pyrazol-1-yl}piperidine-1-carboxylate;

1-methylcyclopropyl 4-(5-cyano-4-{[(2-methylpyridin-3-yl)oxy]methyl}-1H-pyrazol-1-yl)piperidine-1-carboxylate;

isopropyl 4-[5-cyano-4-({[2-methyl-6-(1H-1,2,4-triazol-1-yl)pyridin-3-yl]oxy}methyl)-1H-pyrazol-1-yl]piperidine-1-carboxylate;

isopropyl 4-[5-cyano-4-({[2-methyl-6-(1H-1,2,4-triazol-1-yl)pyridin-3-yl]amino}methyl)-1H-pyrazol-1-yl]piperidine-1-carboxylate;

isopropyl 4-[5-cyano-4-({[2-methyl-6-(methylsulfonyl)pyridin-3-yl]amino}methyl)-1H-pyrazol-1-yl]piperidine-1-carboxylate;

isopropyl 4-{5-cyano-4-[(2-methylphenoxy)methyl]-1H-pyrazol-1-yl}piperidine-1-carboxylate;

isopropyl 4-(5-cyano-4-{[2-fluoro-4-(1-methyl-1H-tetrazol-5-yl)phenoxy]methyl}-1H-pyrazol-1-yl)piperidine-1-carboxylate;

isopropyl 4-(5-cyano-4-{[1-fluoro-4-(2-methyl-2H-tetrazol-5-yl)phenoxy]methyl}-1H-pyrazol-1-yl)piperidine-1-carboxylate;

isopropyl 4-(5-cyano-4-{[(2-methylpyridin-3-yl)amino]methyl}-1H-pyrazol-1-yl)piperidine-1-carboxylate;

isopropyl 4-(5-cyano-4-{1-[(2-methylpyridin-3-yl)oxy]ethyl}-1H-pyrazol-1-yl)piperidine-1-carboxylate;

isopropyl 4-[5-cyano-4-({[2-fluoro-4-(methylsulfonyl)phenyl]amino}methyl)-1H-pyrazol-1-yl]piperidine-1-carboxylate;

isopropyl 4-(5-cyano-4-{1-[2-fluoro-4-(methylsulfonyl)phenoxy]ethyl}-1H-pyrazol-1-yl)piperidine-1-carboxylate;

isopropyl 4-(5-cyano-4-{2-[2-fluoro-4-(methylsulfonyl)phenyl]propyl}-1H-pyrazol-1-yl)piperidine-1-carboxylate;

isopropyl 4-(5-cyano-4-{[2-fluoro-4-(1H-tetrazol-5-yl)phenoxy]methyl}-1H-pyrazol-1-yl)piperidine-1-carboxylate;

isopropyl 4-(5-cyano-4-{2-[2-fluoro-4-(methylsulfonyl)phenyl]ethyl}-1H-pyrazol-1-yl)piperidine-1-carboxylate;

isopropyl 4-{5-cyano-4-[(4-cyano-2-fluorophenoxy)methyl]-1H-pyrazol-1-yl}piperidine-1-carboxylate;

isopropyl 4-(5-cyano-4-{[4-(dimethoxyphosphoryl)-2-fluorophenoxy]methyl}-1H-pyrazol-1-yl)piperidine-1-carboxylate;

isopropyl 4-(5-cyano-4-{[(2-methylpyridin-3-yl)oxy]methyl}-1H-pyrazol-1-yl)piperidine-1-carboxylate;

isopropyl 4-[5-cyano-4-({2-fluoro-4-[(2-hydroxyethyl)sulfonyl]phenoxy}methyl)-1H-pyrazol-1-yl]piperidine-1-carboxylate;

isopropyl 4-(5-cyano-4-{[2-fluoro-4-(1H-tetrazol-1-yl)phenoxy]methyl}-1H-pyrazol-1-yl)piperidine-1-carboxylate;

isopropyl 4-(5-cyano-4-{[4-(1H-tetrazol-1-yl)phenoxy]methyl}-1H-pyrazol-1-yl)piperidine-1-carboxylate;

isopropyl 4-(5-cyano-4-{[2-fluoro-4-(methylsulfonyl)phenoxy]methyl}-1H-pyrazol-1-yl)piperidine-1-carboxylate;

or a pharmaceutically acceptable salt thereof.

The compounds of Formula I modulate the activity of the G-protein-coupled receptor. More specifically the compounds modulate GPR119. As such, said compounds are useful for the treatment of diseases, such as diabetes, in which the activity of GPR119 contributes to the pathology or symptoms of the disease. Examples of such conditions include hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, transient ischemic attacks, stroke, vascular restenosis, hyperglycemia, hyperinsulinemia, hyperlipidemia, hypertriglyceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. The compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

A further embodiment of the invention is directed to pharmaceutical compositions containing a compound of Formula I. Such formulations will typically contain a compound of Formula I in admixture with at least one pharmaceutically acceptable excipient. Such formulations may also contain at least one additional pharmaceutical agent. Examples of such agents include anti-obesity agents and/or anti-diabetic agents. Additional aspects of the invention relate to the use of the compounds of Formula I in the preparation of medicaments for the treatment of diabetes and related conditions as described herein.

It is to be understood that both the foregoing summary and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number:
a. “halogen” refers to a chlorine, fluorine, iodine, or bromine atom.
b. “C₁-C₅ alkyl” refers to a branched or straight chained alkyl group containing from 1 to 5 carbon atoms, such as methyl, ethyl, n-propyl, isopropyl, n-butyl, isobutyl, pentyl, etc.
c. “C₁-C₅ alkoxy” refers to a straight or branched chain alkoxy group containing from 1 to 5 carbon atoms, such as methoxy, ethoxy, n-propoxy, isopropoxy, n-butoxy, isobutoxy, pentoxy, etc.
d. “C₃-C₆ cycloalkyl” refers to a nonaromatic ring that is fully hydrogenated and exists as a single ring. Examples of such carbocyclic rings include cyclopropyl, cyclobutyl, cyclopentyl, and cyclohexyl,
e. “5 to 10 membered heteroaryl” means a carbocyclic aromatic system having a total of 5 to 10 ring atoms and containing one, two, three or four heteroatoms selected independently from oxygen, nitrogen and sulfur and having one, two or three rings wherein such rings may be fused. The term “fused” means that a second ring is present (ie, attached or formed) by having two adjacent atoms in common (ie, shared) with the first ring. The term “fused” is equivalent to the term “condensed”. The term “heteroaryl” embraces aromatic radicals such as pyridine, pyridazine, pyrazine, pyrimidine, imidazo[1,2-a]pyridine, imidazo[1,5-a]pyridine, [1,2,4]triazolo[4,3-a]pyridine, [1,2,4]triazolo[4,3-b]pyridazine, [1,2,4]triazolo[4,3-a]pyrimidine, and [1,2,4]triazolo[1,5-a]pyridine.
f. “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.
g. “patient” refers to warm blooded animals such as, for example, guinea pigs, mice, rats, gerbils, cats, rabbits, dogs, monkeys, chimpanzees, and humans.
h. “treat” embraces both preventative, i.e., prophylactic, and palliative treatment, i.e., relieve, alleviate, or slow the progression of the patient's disease (or condition) or any tissue damage associated with the disease.
i. “the terms “modulated”, “modulating”, or “modulate(s)”, as used herein, unless otherwise indicated, refers to the activation of the G-protein-coupled receptor GPR119 with compounds of the present invention.
j. “pharmaceutically acceptable” indicates that the substance or composition must be compatible chemically and/or toxicologically, with the other ingredients comprising a formulation, and/or the mammal being treated therewith.
k. “salts” is intended to refer to pharmaceutically acceptable salts and to salts suitable for use in industrial processes, such as the preparation of the compound.
l. “pharmaceutically acceptable salts” is intended to refer to either pharmaceutically acceptable acid addition salts” or “pharmaceutically acceptable basic addition salts” depending upon actual structure of the compound.
m. “pharmaceutically acceptable acid addition salts” is intended to apply to any non-toxic organic or inorganic acid addition salt of the base compounds represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
n. “pharmaceutically acceptable basic addition salts” is intended to apply to any non-toxic organic or inorganic basic addition salts of the compounds represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
o. “compound of Formula I”, “compounds of the invention”, and “compounds” are used interchangeably throughout the application and should be treated as synonyms.
p. “isomer” means “stereoisomer” and “geometric isomer” as defined below. “stereoisomer” refers to compounds that possess one or more chiral centers and each center may exist in the R or S configuration. Stereoisomers includes all diastereomeric, enantiomeric and epimeric forms as well as racemates and mixtures thereof. “geometric isomer” refers to compounds that may exist in cis, trans, anti, syn, entgegen (E), and zusammen (Z) forms as well as mixtures thereof.

Certain of the compounds of the Formula (I) may exist as geometric isomers. The compounds of the Formula (I) may possess one or more asymmetric centers, thus existing as two, or more, stereoisomeric forms. The present invention includes all the individual stereoisomers and geometric isomers of the compounds of formula (I) and mixtures thereof. Individual enantiomers can be obtained by chiral separation or using the relevant enantiomer in the synthesis.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention. The compounds may also exist in one or more crystalline states, i.e. as co-crystals, polymorphs, or they may exist as amorphous solids. All such forms are encompassed by the invention and claims.

In one embodiment of the compounds of this invention,

X is

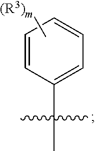

Y is O;

m is 1 or 2;

Z is —C(O)—O—R⁶;

R¹ is hydrogen;

R^{2a} is hydrogen;

R^{2b} is hydrogen; and

each R³ is independently hydroxy, halogen, cyano, CF₃, OCF₃, C₁-C₄ alkyl, C₁-C₄ alkoxy, SO₂—R⁷, —P(O)(OR⁸)(OR⁹), —CO—NR⁸R⁹, or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

In another embodiment of the compounds of this invention,

X is

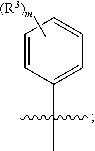

Y is O;

m is 1 or 2;

Z is —C(O)—O—R⁶;

R¹ is hydrogen;

R^{2a} is fluoro;

R^{2b} is hydrogen; and

each R³ is independently hydroxy, halogen, cyano, CF₃, OCF₃, C₁-C₄ alkyl, C₁-C₄ alkoxy, SO₂—R⁷, —P(O)(OR⁸)(OR⁹), —CO—NR⁸R⁹, or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

In another embodiment in the compounds of this invention, each R³ is independently fluoro, methyl, cyano, —C(O)NR⁸R⁹, —SO₂—R⁷, tetrazole, pyrazole, imidazole or triazole.

In another embodiment in the compounds of this invention, each R³ is independently fluoro, methyl, cyano, —C(O)NR⁸R⁹, —SO₂—R⁷,

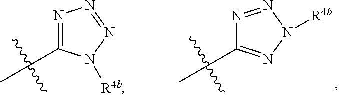

, or

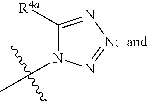

R^{4a} and R^{4b} are each independently hydrogen, C₁-C₄ alkyl, or C₂-C₄ alkyl-OH.

In another embodiment in the compounds of this invention,

X is

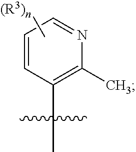

Y is O or NH;

Z is —C(O)—O—R⁶,

n is 0 or 1;

R¹ is hydrogen;

R^{2a} is hydrogen;

R^{2b} is hydrogen; and

R³, if present, is C₁-C₄ alkyl or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

In another embodiment in the compounds of this invention,

X is

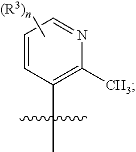

Y is O or NH;

Z is —C(O)—O—R⁶;

n is 0 or 1;

R¹ is hydrogen;

R^{2a} is fluoro;

R^{2b} is hydrogen; and

R³, if present, is C₁-C₄ alkyl or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

In another embodiment in the compounds of this invention, R⁶ is isopropyl or 1-methylcyclopropyl.

In another embodiment in the composition of this invention, the composition further includes at least one additional pharmaceutical agent selected from the group consisting of an anti-obesity agent and an anti-diabetic agent. Example anti-obesity agents include dirlotapide, mitratapide, implitapide, R56918 (CAS No. 403987), CAS No. 913541-47-6, lorcaserin, cetilistat, PYY_{3-36,} naltrexone, oleoyl-estrone, obinepitide, pramlintide, tesofensine, leptin, liraglutide, bromocriptine, orlistat, exenatide, AOD-9604 (CAS No. 221231-10-3) and sibutramine. Example anti-diabetic agents include metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

In another embodiment of the method of this invention, the compounds or compositions of this invention may be administered in an effective amount for treating a condition selected from the group consisting of hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome.

In a further embodiment, the method further includes administering a second composition comprising at least one additional pharmaceutical agent selected from the group consisting of an anti-obesity agent and an anti-diabetic agent, and at least one pharmaceutically acceptable excipient. This method may be used for administering the compositions simultaneously or sequentially and in any order.

In yet another embodiment, the compounds of this invention are useful in the manufacture of a medicament for treating a disease, condition or disorder that modulates the activity of G-protein-coupled receptor GPR119. Furthermore, the compounds are useful in the preparation of a medicament for the treatment of diabetes or a morbidity associated with said diabetes.

### Synthesis

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database).

The compounds of Formula I can be prepared using methods analogously known in the art for the production of ethers. The reader's attention is directed to texts such as: 1) Hughes, D. L.; Organic Reactions 1992, 42 Hoboken, N.J., United States; 2) Tikad, A.; Routier, S.; Akssira, M.; Leger, J.-M. I; Jarry, C.; Guillaumet, G. Synlett 2006, 12, 1938-42; and 3) Loksha, Y. M.; Globisch, D.; Pedersen, E. B.; La Colla, P.; Collu, G.; Loddo, R. J. Het. Chem. 2008, 45, 1161-6 which describe such reactions in greater detail.

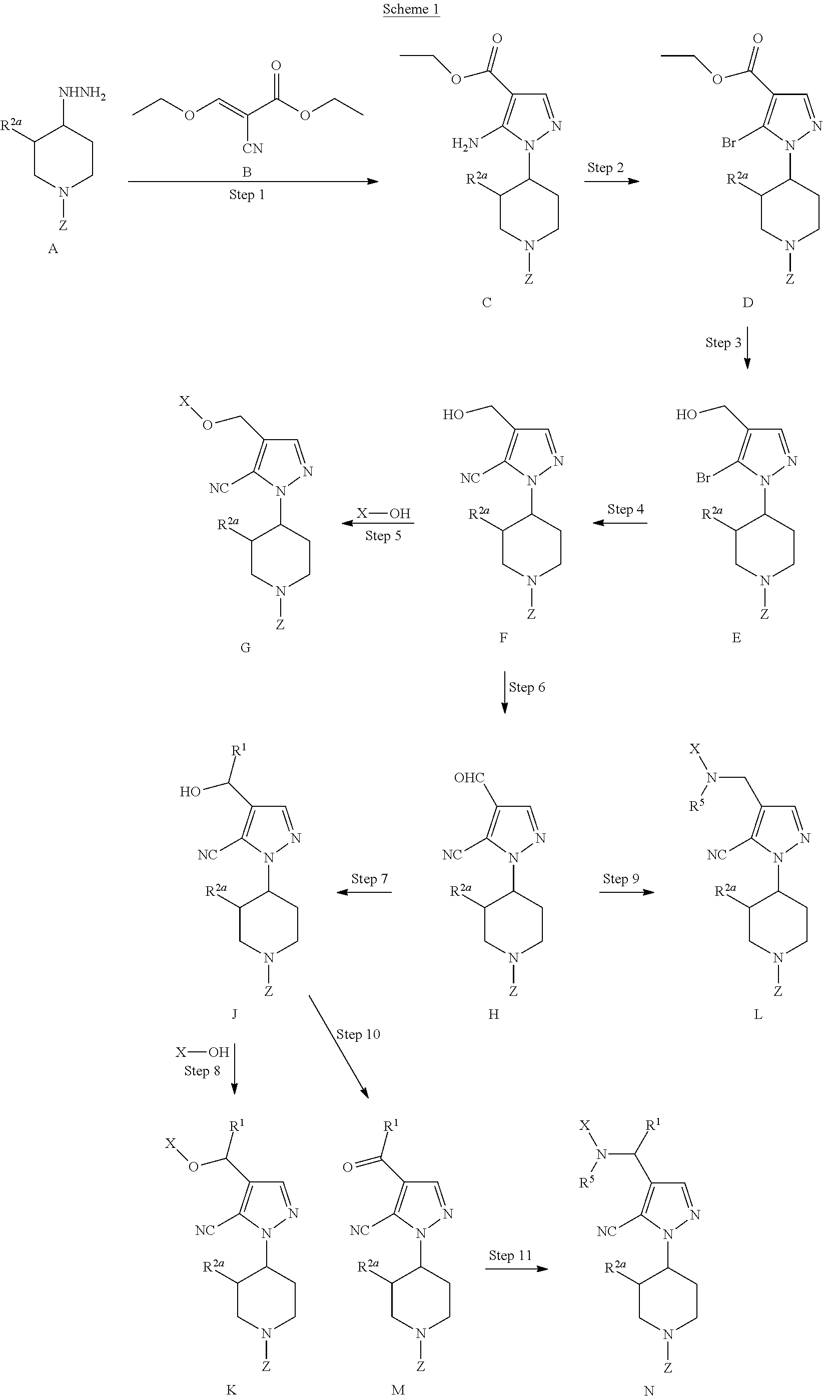

Compounds of Formula I wherein R^{2b} is H, may be prepared as shown in Scheme 1. In Step 1, compounds of Formula C can be prepared via a condensation reaction of compounds of Formula A and the commercial compound B (Sigma-Aldrich) in a diverse array of solvents including but not limited to ethanol, toluene and acetonitrile at temperatures ranging from 22° C. to 130° C. depending upon the solvent utilized for a period of 1 to 72 hours. In cases where compounds of Formula A are hydrogen chloride or trifluoroacetic acid salts, base modifiers such as sodium acetate or sodium bicarbonate may be added in one to three equivalents to neutralize the salts. The reaction may be conducted in polar protic solvents such as methanol and ethanol at temperatures ranging from 22° C. to 85° C. Typical conditions for this transformation include the use of 3 equivalents of sodium acetate in ethanol heated at 85° C. for 3 hours.

Compounds of Formula A can be prepared via a four-step procedure starting with substituted or unsubstituted 4-piperidinone hydrochloride salts (J. Med. Chem. 2004, 47, 2180). First these salts are treated with an appropriate alkyl chloroformate or bis(alkyl) IS dicarbonate in the presence of excess base to form the corresponding alkyl carbamate. The ketone group is then condensed with tert-butoxycarbonyl hydrazide to form the corresponding N-(tert-butoxy)carbonyl (BOC) protected hydrazone derivative. This is subsequently reduced to the corresponding BOC protected hydrazine derivative using reducing agents such as sodium cyanoborohydride or sodium triacetoxyborohydride. Finally, the N-(tert-butoxy)carbonyl group is cleaved under acidic conditions such as trifluoroacetic acid or hydrochloric acid to give compounds of Formula A, which are typically isolated and used as the corresponding salts (e.g., dihydrochloride salt).

In Step 2, compounds of Formula D may be prepared from compounds of Formula C via the formation of intermediate diazonium salts via the Sandmeyer reaction (Comp. Org. Synth., 1991, 6, 203) These salts may be prepared via diazotization of compounds of Formula C with sodium nitrite and aqueous acids such as hydrochloric, hydrobromic, sulfuric, nitric, phosphoric and acetic alone or in combinations. This reaction is typically carried out in water at 0° C. to 100° C. Alternatively, anhydrous conditions using alkyl nitrites such as tert-butylnitrite with solvents such as acetonitrile may be utilized (J. Med. Chem. 2006, 49, 1562) at temperatures ranging from 0° C. to 95° C. These diazonium intermediates are then allowed to react with copper salts such as copper(II) bromide, copper(I) bromide or with tribromomethane to form compounds of Formula D. Typical conditions for this transformation include the use of tert-butylnitrite, copper(II) bromide in acetonitrile at 65° C. for 30 minutes.

In Step 3, compounds of Formula E may be prepared from compounds of Formula D via the use of reducing agents such as lithium aluminum hydride, sodium borohydride, lithium borohydride, borane-dimethylsulfide, borane-tetrahydrofuran in polar aprotic solvents such as tetrahydrofuran, diethyl ether, 1,4-dioxane or 1,2-dimethoxyethane at temperatures ranging from 0° C. to 110° C. for 1 to 24 hours. Typical conditions include the use of borane-dimethylsulfide in tetrahydrofuran at 70° C. for 14 hours.

In order to prepare compounds of Formula F from compounds of Formula E, a cyano group must be introduced (Step 4) This may be achieved via a range of conditions. One method of cyano group introduction may be the use of a copper salt such as copper cyanide in a polar aprotic solvent such as N,N-dimethylformamide (DMF), N-methylpyrrolidinone (NMP), N,N-dimethylacetamide (DMA) at temperatures ranging from 22° C. to 200° C. for 1 to 24 hours. Copper cyanide in N,N-dimethylformamide heated at 165° C. for 5 hours is a typical protocol for this transformation.

Alternatively in Step 4, alkali cyanide salts such as potassium or sodium cyanide may be used in conjunction with catalysts such as 18-crown-6 (US2005020564) and or tetrabutylammonium bromide (J. Med. Chem. 2003, 46, 1144) in polar aprotic solvents such acetonitrile and dimethylsulfoxide at temperatures ranging from 22° C. to 100° C. for the addition of a cyano group to this template.

Finally, the use of metal catalysis is common for the transformation depicted in Step 4. Common cyanide salts used in catalytic procedures include zinc cyanide, copper cyanide, sodium cyanide, and potassium hexacyanoferrate (II). The metal catalysts can be copper catalysts such as copper iodide and or palladium catalysts such as tris(dibenzylideneacetone)dipalladium (Pd₂(dba)₃), palladium tetrakis-triphenylphosphine (Pd(PPh₃)₄), or dichloro(diphenyl-phosphinoferrocene)-palladium (Pd(dppf)Cl₂). These catalysts may be used alone or in any combination with any of the above cyanide salts. To these reactions may be added ligands such as 1,1′-bis(diphenylphosphino)-ferrocene (dppf) or metal additives such as zinc or copper metal. The reactions are carried out in polar aprotic solvents such as NMP, DMF, DMA with or without water as an additive. The reactions are carried out at temperatures ranging from 22° C. to 150° C. via conventional or microwave heating for 1 to 48 hours and may be conducted in a sealed or non-sealed reaction vessel. Typical conditions for Step 4 include the use of zinc cyanide, Pd₂(dba)₃, dppf, and zinc dust in DMA heated at 120° C. in a microwave for 1 hour (J. Med. Chem. 2005, 48, 1132).

In Step 5, compounds of Formula G, wherein X, Z and R^{2a} are as defined for compounds of Formula I, can be synthesized from compounds of Formula F via the Mitsunobu reaction. The Mitusunobu reaction has been reviewed in the synthetic literature (e.g., Chem. Asian. J. 2007, 2, 1340; Eur. J. Org. Chem. 2004, 2763; S. Chem. Eur. J. 2004, 10, 3130), and many of the synthetic protocols listed in these reviews may be used. The use of Mitsunobu reaction protocols utilizing azodicarboxylates such as diethyl azodicarboxylate (DEAD), di-tert-butyl azodicarboxylate (TBAD), diisopropyl azodicarboxylate (DIAD) and a phosphine reagent such as triphenylphosphine (PPh₃), tributylphoshine (PBu₃) and polymer supported triphenylphosphine (PS-PPh₃) are combined with compounds of Formula F and a compound of general structure X—OH, wherein X is as defined for compounds of Formula I. Solvents utilized in this reaction may include aprotic solvents such as toluene, benzene, THF, 1,4-dioxane and acetonitrile at temperatures ranging from 0° C. to 130° C. depending on the solvent and azodicarboxylates utilized. Typical conditions for this transformation are the use of DEAD with PS-PPh₃ in 1,4-dioxane at 22° C. for 15 hours.

An alternative to the Mitsunobu reaction for preparing compounds of Formula G, wherein X, Z, and R^{2a} are as defined for compounds of Formula I, is to convert the compounds of Formula F to the corresponding methanesulfonate or para- toluenesulphonate derivatives using methanesulfonyl chloride or para-toluenesulfonyl chloride, respectively, in the presence of a base such as triethylamine or pyridine. The intermediate sulfonate ester is then combined with a compound of general X—OH, wherein X is as defined for compounds of Formula I, in the presence of a base such as potassium carbonate, sodium hydride, or potassium tert-butoxide to yield compounds of Formula G, wherein X, Z, and R^{2a} are as defined for compounds of Formula I.

Compounds of Formula K, wherein R¹ is C₁-C₄ alkyl or C₃-C₆ cycloalkyl and X, Z and R^{2a} are as defined for compounds of Formula I, may be prepared from compounds of Formula F in three Steps: 1) oxidation of the primary alcohol to the corresponding aldehyde of Formula H (Step 6, Scheme 1), 2) reaction of the aldehyde intermediate of Formula H with an organometallic reagent of the Formula R¹M, wherein M is lithium (Li) or magnesium halide (MgCl, MgBr or MgI) to provide a secondary alcohol of Formula J, wherein R¹ is C₁-C₄ alkyl or C₃-C₆ cycloalkyl (Step 7), and 3) reaction of the secondary alcohol of Formula J with a phenol of the Formula X—OH, wherein X is as defined for compounds of Formula I, under Mitsunobu reaction conditions (Step 8).

In Step 6 (Scheme 1), compounds of Formula H can are formed via oxidation procedures including the use of 1 to 20 equivalents of activated manganese dioxide in solvents including but not limited to dichloromethane, acetonitrile, hexane or acetone alone or in combinations for 1 to 72 hours at 22° C. to 80° C. Alternatively, this oxidation can be conducted with 1 to 3 equivalents of trichloroisocyanuric acid in the presence of 0.1 to 1 equivalents of 2,2,6,6-tetramethylpiperidine-1-oxyl (TEMPO) in dichloromethane or chloroform at temperatures ranging from 0° C. to 22° C. for 0.1 to 12 hours. Typical conditions for this transformation are the use of trichloroisocyanuric acid in the presence of 0.1 equivalent of TEMPO in dichloromethane at 22° C. for 1 hour.

The preparation of compounds of Formula I wherein Y is NR⁵ is also shown in Scheme 1. Compounds of Formula L wherein X, Z, R^{2a} and R⁵ are as defined for compounds of Formula I may be prepared from the intermediate compound of Formula H (Scheme 1) by reaction with an amino compound of the Formula X—NH—R⁵, wherein X and R⁵ are as defined for compounds of Formula I, under reductive amination conditions (Step 9) (J. Org. Chem., 1996, 61, 3849; Org. React. 2002, 59, 1). Similarly compounds of Formula N, wherein R¹ is C₁-C₄ alkyl or C₃-C₆ cycloalkyl and X, Z, R^{2a} and R⁵ are as defined for compounds of Formula I, may be prepared in two steps from the intermediate of Formula J wherein R¹ is C₁-C₄ alkyl or C₃-C₆ cycloalkyl, by 1) oxidation to the corresponding ketone of Formula M (Step 10), and 2) reaction of the ketone of Formula M with an amino compound of the Formula X—NH—R⁵, wherein X and R⁵ are as defined for compounds of-Formula I, under reductive amination conditions (Step 11). Alternatively compounds of Formula L and Formula N, wherein R⁵ is C₁-C₄ alkyl may be prepared from the corresponding compounds of Formula L, wherein R⁵ is H, or the corresponding compounds of Formula N, wherein R⁵ is H, by alkylation with an alkyl halide of Formula (C₁-C₄)—Cl, (C₁-C₄)—Br or (C₁-C₄)—I in the presence of a base.

Compounds of Formula I wherein Y is CHR⁵ and R^{2b} is hydrogen may be prepared as shown in Schemes 2 and 3. Compounds of Formula R, wherein X, Z and R^{2a} are as defined for compounds of Formula I may be prepared as shown in Scheme 2.

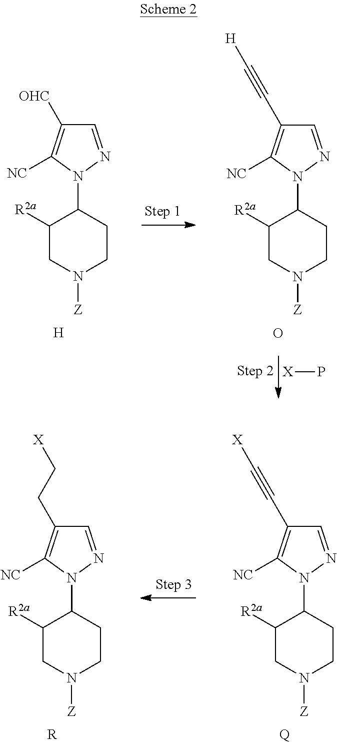

In Step 1 of Scheme 2, compounds of the Formula O can be formed from aldehydes of Formula H (see also Scheme 1) via the use of either dimethyl(diazomethyl)phosphonate or dimethyl-1-diazo-2-oxopropylphosphonate and bases such as potassium carbonate or potassium tert-butoxide in solvents including methanol, ethanol or tetrahydrofuran at temperatures ranging from −78° C. to 22° C. for 0.1 to 24 hours. Typical conditions for this transformation include the use of dimethyl-1-diazo-2-oxopropylphosphonate and 2 equivalents of potassium carbonate in methanol at 22° C. for 0.75 hour.

In Step 2, compounds of Formula Q can be formed from compounds of Formula O via a metal-catalyzed Sonagashira coupling procedure with compounds of general structure X—P wherein X is as defined for compounds of Formula I and P is a halide or trifluoromethsulfonate(triflate). The Sonogashira reaction has been extensively reviewed (Chem. Rev. 2007, 107, 874; Angew. Chem. Int. Ed. 2007, 46, 834; Angew. Chem. Int. Ed. 2008, 47, 6954), and many of the synthetic protocols listed in these reviews may be used for the synthesis of compounds of Formula Q. Typically, the use of metal catalysts in this reaction can be copper catalysts such as copper iodide and or palladium catalysts such as Pd₂(dba)₃, Pd(PPh₃)₄, Pd(dppf)Cl₂ or Pd(PPh₃)₂Cl₂. These catalysts may be used alone or in any combination. Base additives are typically used in this reaction and may include amine bases such as diethylamine, triethylamine, diisopropylethylamine or pyrrolidine or inorganic bases such as potassium carbonate or potassium fluoride. The reactions are carried out in solvents such as dichloromethane, chloroform, acetonitrile, DMF, toluene or 1,4-dioxane with or without water as an additive. The reactions are carried out at temperatures ranging from 0° C. to 150° C. depending on the solvent for times ranging from 0.1 to 48 hours. Typical conditions for this transformation include the use of CuI and Pd(PPh₃)₂Cl₂ in DMF at 90° C. for 2 hours.

Finally, in Step 3 compounds of Formula R, wherein X, Z and R^{2a} are as defined for compounds of Formula I, can be formed from compounds of Formula Q via hydrogenation in the presence of transition metal catalysts. Common catalysts include the use of 5-20% palladium on carbon or 5-20% palladium hydroxide on carbon. These reactions can be conducted in a Parr shaker apparatus or in an H-Cube hydrogenation flow reactor (ThalesNano, U.K.) under pressures of hydrogen ranging from 1 to 50 psi in polar solvents such as tetrahydrofuran, ethyl acetate, methanol or ethanol at temperatures of 22° C. to 50° C. for times ranging from 0.1 to 24 hours. Typical conditions for Step 3 include the use compound of Formula Q in ethyl acetate at a flow rate of 1 mL/min through a 10% palladium on carbon cartridge on the H-Cube flow apparatus set at the “full hydrogen” setting.

Scheme 3 shows methods for the preparation of compounds of Formula W, wherein X, Z, R^{2a} and R⁵ are as defined for compounds of Formula I.

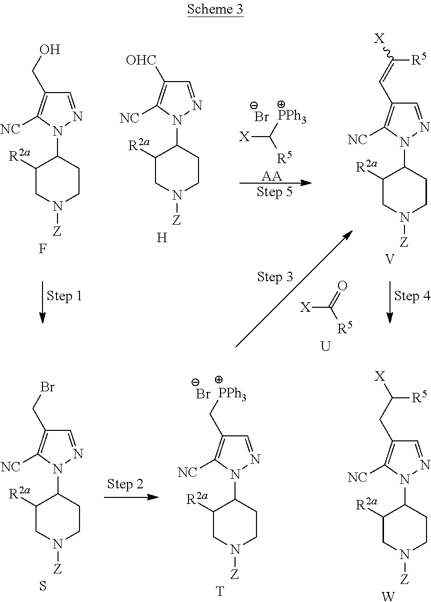

In Step 1 of Scheme 3, compounds of Formula F (see also Scheme 2) can be treated with reagents such as phosphorus tribromide or carbon tetrabromide and triphenylphosphine to give compounds of Formula S. In Step 2, compounds of Formula S are then allowed to react with triphenylphosphine in solvents such as dichloromethane, chloroform, toluene, benzene, tetrahydrofuran (THF) or acetonitrile to give triphenylphosphonium salts of Formula T. The salts of Formula T, are then combined with carbonyl compounds of Formula U, where X and R⁵ are as defined for compounds of Formula I, in the presence of bases such as n-butyllithium, sodium bis(trimethylsilyl)amide, lithium bis(trimethylsilyl)amide, potassium bis(trimethylsilyl)amide or lithium diisopropylamide in solvents such as THF, diethylether or 1,4-dioxane, to yield alkene compounds of Formula V, which are typically isolated as mixtures of E and Z geometric isomers (Step 3). This reaction, commonly known as the Wittig olefination reaction, has been reviewed extensively in the literature (Chem. Rev. 1989, 89, 863; Modern Carbonyl Olefination 2004, 1-17; Liebigs Ann. Chem. 1997, 1283).

In Step 4, compounds of Formula W, wherein X, Z, R^{2a} and R⁵ are as defined for compounds of Formula I, are formed from compounds of Formula V via hydrogenation in the presence of transition metal catalysts. Common catalysts include the use of 5-20% palladium on carbon or 5-20% palladium hydroxide on carbon. These reactions can be conducted in a similar manner as described for Step 3 of Scheme 2.

Alternatively compounds of Formula W, wherein X, Z, and R^{2a} are as defined for compounds of Formula I, may be prepared from aldehydes of Formula H via Wittig reaction with triphenylphosphonium salts of Formula AA (Step 5, Scheme 3). As for Step 3, this reaction produces alkene compounds of Formula V, which again are typically isolated as mixtures of E and Z geometric isomers, and may be converted to compounds of Formula W, wherein X, Z, R^{2a} and R⁵ are as defined for compounds of Formula I, by hydrogenation. The salts of Formula AA are obtained in a similar manner to that used for preparing salts of Formula T via conversion of the corresponding alcohol to the bromide and subsequent reaction with triphenylphosphine.

Compounds of Formula BB shown below, wherein X, Z, R¹ and R^{2a} are as defined for compounds of Formula I, can be prepared from secondary alcohols of Formula J (see Scheme 2) or ketones of Formula M (see Scheme 2) through reaction sequences similar to those shown in Scheme 3. Conversion of compounds of Formula J to the corresponding bromides, followed by Wittig olefination with aldehydes of general formula X—CHO, wherein X is as defined for compounds of Formula I, provides alkenes of Formula CC. Alkenes of Formula CC may also be obtained via Wittig reaction of ketones of Formula M with salts of the general structure X—CH₂—PPh₃⁺Br⁻. The alkenes of Formula CC are then converted to compounds of Formula BB, wherein X, Z, R¹ and R^{2a} are as defined for compounds of Formula I, by hydrogenation.

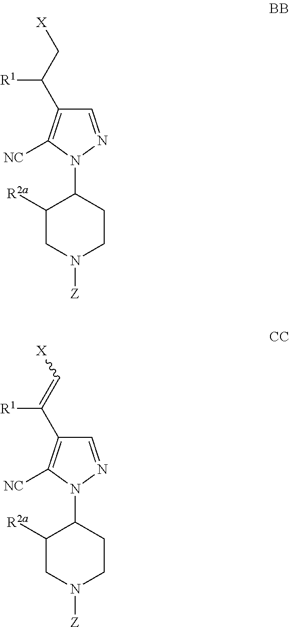

In certain instances it is possible to change the order of steps shown in Schemes 1, 2 and 3. For example, in Scheme 1, it is sometimes possible to introduce the cyano group on the pyrazole ring as the last step, i.e., inverting the order in which Steps 4 and 5 are carried out. Also, in certain cases, it is preferable to introduce or modify substituents R³ on the group X (wherein R³ and X are as defined for compounds of Formula I) later in the synthesis, even as the last step. For example, when R³ is SO₂R⁷, the SO₂R⁷ group may be in formed in the last step by oxidation of the corresponding compound bearing a substituent of general formula S—R⁷.

Compounds of Formula I wherein R^{2a} and R^{2b} are fluoro may be prepared according to sequences analogous to those shown in Schemes 1, 2 and 3 starting with 3,3-difluoro-4,4-dihydroxy 1-piperidine carboxylic acid 1,1-dimethylethyl ester (WO 2008121687). In a manner similar to that described for the preparation of intermediates of formula A in Scheme 1, this material may be converted to hydrazine derivatives of formula DD, which are then used similarly to the intermediates of formula A in Scheme 1 for the preparation of compounds of Formula I wherein R^{2a} and R^{2-b} are fluoro.

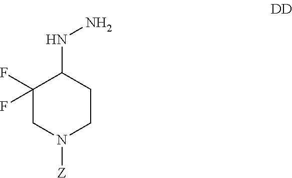

As is readily apparent to one skilled in the art, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBZ) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

As noted above, some of the compounds of this invention are acidic and they form salts with pharmaceutically acceptable cations. Some of the compounds of this invention are basic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures

As noted above, some of the compounds exist as isomers. These isomeric mixtures can be separated into their individual isomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Certain isotopically labeled ligands including tritium, ¹⁴C, ³⁵S and ¹²⁵I could be useful in radioligand binding assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

### Medical Uses

Compounds of the present invention modulate the activity of G-protein-coupled receptor GPR119. As such, said compounds are useful for the prophylaxis and treatment of diseases, such as diabetes, in which the activity of GPR119 contributes to the pathology or symptoms of the disease. Consequently, another aspect of the present invention includes a method for the treatment of a metabolic disease and/or a metabolic-related disorder in an individual which comprises administering to the individual in need of such treatment a therapeutically effective amount of a compound of the invention, a salt of said compound or a pharmaceutical composition containing such compound. The metabolic diseases and metabolism-related disorders are selected from, but not limited to, hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations, endothelial dysfunction, hyper apo B lipoproteinemia and impaired vascular compliance. Additionally, the compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

In accordance with the foregoing, the present invention further provides a method for preventing or ameliorating the symptoms of any of the diseases or disorders described above in a subject in need thereof, which method comprises administering to a subject a therapeutically effective amount of a compound of the present invention. Further aspects of the invention include the preparation of medicaments for the treating diabetes and its related co-morbidities.

In order to exhibit the therapeutic properties described above, the compounds need to be administered in a quantity sufficient to modulate activation of the G-protein-coupled receptor GPR119. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the present invention may be administered by a variety of routes. They may be administered orally. The compounds may also be administered parenterally (i.e., subcutaneously, intravenously, intramuscularly, intraperitoneally, or intrathecally), rectally, or topically.

### Co-Administration

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a diacylglycerol O-acyltransferase 1 (DGAT-1) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an a-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, and a SGLT2 inhibitor (sodium dependent glucose transporter inhibitors such as dapagliflozin, etc). Preferred anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆ (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

All of the above recited U.S. patents and publications are incorporated herein by reference.

### Pharmaceutical Formulations

The present invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington“s Pharmaceutical Sciences, 21^{st} Edition, by University of the Sciences in Philadelphia.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### Examples

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England), Mallinckrodt Baker (Phillipsburg N.J.); EMD (Gibbstown, N.J.).

### General Experimental Procedures

NMR spectra were recorded on a Varian Unity^{SM} 400 (DG400-5 probe) or 500 (DG500-5 probe—both available from Varian Inc., Palo Alto, Calif.) at room temperature at 400 MHz or 500 MHz respectively for proton analysis. Chemical shifts are expressed in parts per million (delta) relative to residual solvent as an internal reference. The peak shapes are denoted as follows: s, singlet; d, doublet; dd, doublet of doublet; t, triplet; q, quartet; m, multiplet; bs, broad singlet; 2s, two singlets.

Atmospheric pressure chemical ionization mass spectra (APCI) were obtained on a Waters™ Spectrometer (Micromass ZMD, carrier gas: nitrogen) (available from Waters Corp., Milford, Mass., USA) with a flow rate of 0.3 mL/minute and utilizing a 50:50 water/acetonitrile eluent system. Electrospray ionization mass spectra (ES) were obtained on a liquid chromatography mass spectrometer from Waters™ (Micromass ZQ or ZMD instrument (carrier gas: nitrogen) (Waters Corp., Milford, Mass., USA) utilizing a gradient of 95:5-0:100 water in acetonitrile with 0.01% formic acid added to each solvent. These instruments utilized a Varian Polaris 5 C18-A20×2.0 mm column (Varian Inc., Palo Alto, Calif.) at flow rates of 1 mL/minute for 3.75 minutes or 2 mL/minute for 1.95 minutes.

Column chromatography was performed using silica gel with either Flash 40 Biotage™ columns (ISC, Inc., Shelton, Conn.) or Biotage™ SNAP cartridge KPsil or Redisep Rf silica (from Teledyne Isco Inc) under nitrogen pressure. Preparative HPLC was performed using a Waters FractionLynx system with photodiode array (Waters 2996) and mass spectrometer (Waters/Micromass ZQ) detection schemes. Analytical HPLC work was conducted with a Waters 2795 Alliance HPLC or a Waters ACQUITY UPLC with photodiode array, single quadrupole mass and evaporative light scattering detection schemes.

Concentration in vacuo refers to evaporation of solvent under reduced pressure using a rotary evaporator.

Unless otherwise noted, chemical reactions were performed at room temperature (about 23 degrees Celsius). Also, unless otherwise noted chemical reactions were run under an atmosphere of nitrogen.

### Pharmacological Data

The practice of the invention for the treatment of diseases modulated by the agonist activation of G-protein-coupled receptor GPR119 with compounds of the invention can be evidenced by activity in one or more of the functional assays described herein below. The source of supply is provided in parenthesis.

### In-Vitro Functional Assays

β-lactamase:

The assay for GPR119 agonists utilizes a cell-based (hGPR119 HEK293-CRE beta-lactamase) reporter construct where agonist activation of human GPR119 is coupled to beta-lactamase production via a cyclic AMP response element (CRE). GPR119 activity is then measured utilizing a FRET-enabled beta-lactamase substrate, CCF4-AM (Live Blazer FRET-B/G Loading kit, Invitrogen cat #K1027). Specifically, hGPR119-HEK-CRE-beta-lactamase cells (Invitrogen 2.5×10⁷/mL) were removed from liquid nitrogen storage, and diluted in plating medium (Dulbecco's modified Eagle medium high glucose (DMEM; Gibco Cat #11995-065), 10% heat inactivated fetal bovine serum (HIFBS; Sigma Cat #F4135), 1× MEM Nonessential amino acids (Gibco Cat #15630-080), 25 mM HEPES pH 7.0 (Gibco Cat #15630-080), 200 nM potassium clavulanate (Sigma Cat #P3494). The cell concentration was adjusted using cell plating medium and 50 microL of this cell suspension (12.5×10⁴ viable cells) was added into each well of a black, clear bottom, poly-d-lysine coated 384-well plate (Greiner Bio-One cat #781946) and incubated at 37 degrees Celsius in a humidified environment containing 5% carbon dioxide. After 4 hours the plating medium was removed and replaced with 40 microL of assay medium (Assay medium is plating medium without potassium clavulanate and HIFBS). Varying concentrations of each compound to be tested was then added in a volume of 10 microL (final DMSO≦0.5%) and the cells were incubated for 16 hours at 37 degrees Celsius in a humidified environment containing 5% carbon dioxide. Plates were removed from the incubator and allowed to equilibrate to room temperature for approximately 15 minutes. 10 microL of 6×CCF4/AM working dye solution (prepared according to instructions in the Live Blazer FRET-B/G Loading kit, Invitrogen cat #K1027) was added per well and incubated at room temperature for 2 hours in the dark. Fluorescence was measured on an EnVision fluorimetric plate reader, excitation 405 nm, emission 460 nm/535 nm. EC₅₀ determinations were made from agonist-response curves analyzed with a curve fitting program using a 4-parameter logistic dose-response equation.

cAMP:

GPR119 agonist activity was also determined with a cell-based assay utilizing an HTRF (Homogeneous Time-Resolved Fluorescence) cAMP detection kit (cAMP dynamic 2 Assay Kit; Cis Bio cat #62AM4PEC) that measures cAMP levels in the cell. The method is a competitive immunoassay between native cAMP produced by the cells and the cAMP labeled with the dye d2. The tracer binding is visualized by a Mab anti-cAMP labeled with Cryptate. The specific signal (i.e. energy transfer) is inversely proportional to the concentration of cAMP in either standard or sample.

Specifically, hGPR119 HEK-CRE beta-lactamase cells (Invitrogen 2.5×10⁷/mL; the same cell line used in the beta-lactamase assay described above) are removed from cryopreservation and diluted in growth medium (Dulbecco's modified Eagle medium high glucose (DMEM; Gibco Cat #11995-065), 1% charcoal dextran treated fetal bovine serum (CD serum; HyClone Cat #SH30068.03), 1× MEM Nonessential amino acids (Gibco Cat #15630-080) and 25 mM HEPES pH 7.0 (Gibco Cat #15630-080)). The cell concentration was adjusted to 1.5×10⁵ cells/mL and 30 mLs of this suspension was added to a T-175 flask and incubated at 37 degrees Celsius in a humidified environment in 5% carbon dioxide. After 16 hours (overnight), the cells were removed from the T-175 flask (by rapping the side of the flask), centrifuged at 800×g and then re-suspended in assay medium (1× HBSS+CaCl₂+MgCl₂ (Gibco Cat #14025-092) and 25 mM HEPES pH 7.0 (Gibco Cat #15630-080)). The cell concentration was adjusted to 6.25×10⁵ cells/mL with assay medium and 8 μl of this cell suspension (5000 cells) was added to each well of a white Greiner 384-well, low-volume assay plate (VWR cat #82051-458).

Varying concentrations of each compound to be tested were diluted in assay buffer containing 3-isobutyl-1-methylxanthin (IBMX; Sigma cat #I5879) and added to the assay plate wells in a volume of 2 microL (final IBMX concentration was 400 microM and final DMSO concentration was 0.58%). Following 30 minutes incubation at room temperature, 5 microL of labeled d2 cAMP and 5 microL of anti-cAMP antibody (both diluted 1:20 in cell lysis buffer; as described in the manufacturers assay protocol) were added to each well of the assay plate. The plates were then incubated at room temperature and after 60 minutes, changes in the HTRF signal were read with an Envision 2104 multilabel plate reader using excitation of 330 nm and emissions of 615 and 665 nm. Raw data were converted to nM cAMP by interpolation from a cAMP standard curve (as described in the manufacturer's assay protocol) and EC50 determinations were made from an agonist-response curves analyzed with a curve fitting program using a 4-parameter logistic dose response equation.

It is recognized that cAMP responses due to activation of GPR119 could be generated in cells other than the specific cell line used herein.

### β-Arrestin:

GPR119 agonist activity was also determined with a cell-based assay utilizing DiscoverX PathHunter β-arrestin cell assay technology and their U2OS hGPR119 β-arrestin cell line (DiscoverX Cat #93-0356C3). In this assay, agonist activation is determined by measuring agonist-induced interaction of 13-arrestin with activated GPR119. A small, 42 amino acid enzyme fragment, called ProLink was appended to the C-terminus of GPR119. Arrestin was fused to the larger enzyme fragment, termed EA (Enzyme Acceptor). Activation of GPR119 stimulates binding of arrestin and forces the complementation of the two enzyme fragments, resulting in formation of a functional β-galactosidase enzyme capable of hydrolyzing substrate and generating a chemiluminescent signal.

Specifically, U2OS hGPR119 β-arrestin cells (DiscoverX 1×10⁷/mL) are removed from cryopreservation and diluted in growth medium (Minimum essential medium (MEM; Gibco Cat #11095-080), 10% heat inactivated fetal bovine serum (HIFBS; Sigma Cat #F4135-100), 100 mM sodium pyruvate (Sigma Cat #S8636), 500 microg/mL G418 (Sigma Cat #G8168) and 250 microg/mL Hygromycin B (Invitrogen Cat #10687-010). The cell concentration was adjusted to 1.66×10⁵ cells/mL and 30 mLs of this suspension was added to a T-175 flask and incubated at 37 degrees Celsius in a humidified environment in 5% carbon dioxide. After 48 hours, the cells were removed from the T-175 flask with enzyme-free cell dissociation buffer (Gibco cat #13151-014), centrifuged at 800×g and then re-suspended in plating medium (Opti-MEM I (Invitrogen/BRL Cat #31985-070) and 2% charcoal dextran treated fetal bovine serum (CD serum; HyClone Cat #SH30068.03). The cell concentration was adjusted to 2.5×10⁵ cells/mL with plating medium and 10 microL of this cell suspension (2500 cells) was added to each well of a white Greiner 384-well low volume assay plate (VWR cat #82051-458) and the plates were incubated at 37 degrees Celsius in a humidified environment in 5% carbon dioxide.

After 16 hours (overnight) the assay plates were removed from the incubator and varying concentrations of each compound to be tested (diluted in assay buffer (1× HBSS+CaCl₂+MgCl₂ (Gibco Cat #14025-092), 20 mM HEPES pH 7.0 (Gibco Cat #15630-080) and 0.1% BSA (Sigma Cat #A9576)) were added to the assay plate wells in a volume of 2.5 microL (final DMSO concentration was 0.5%). After a 90 minute incubation at 37 degrees Celsius in a humidified environment in 5% carbon dioxide, 7.5 microL of Galacton Star β-galactosidase substrate (PathHunter Detection Kit (DiscoveRx Cat #93-0001); prepared as described in the manufacturers assay protocol) was added to each well of the assay plate. The plates were incubated at room temperature and after 60 minutes, changes in the luminescence were read with an Envision 2104 multilabel plate reader at 0.1 seconds per well. EC50 determinations were made from an agonist-response curves analyzed with a curve fitting program using a 4-parameter logistic dose response equation.

### Expression of GPR119 using BacMam and GPR119 Binding Assay

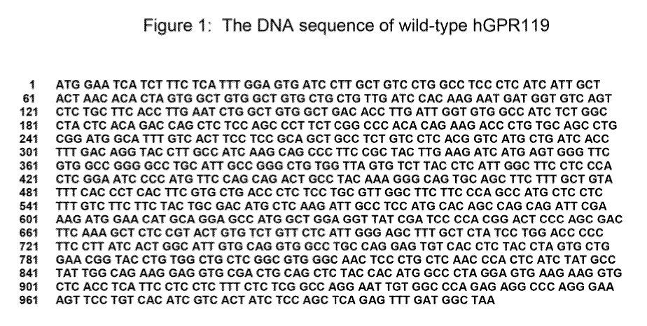

Wild-type human GPR119 (FIG. 1) was amplified via polymerase chain reaction (PCR) (Pfu Turbo Mater Mix, Stratagene, La Jolla, Calif.) using pIRES-puro-hGPR119 as a template and the following primers:

hGPR119 BamH1, Upper
5′-TAAATTGGATCCACCATGGAATCATCTTTCTCATTTGGAG-3′
(inserts a BamHI site at the 5′ end)
hGPR119 EcoRI, Lower
5′-TAAATTGAATTCTTATCAGCCATCAAACTCTGAGC-3′
(inserts a EcoRI site at the 3′ end)

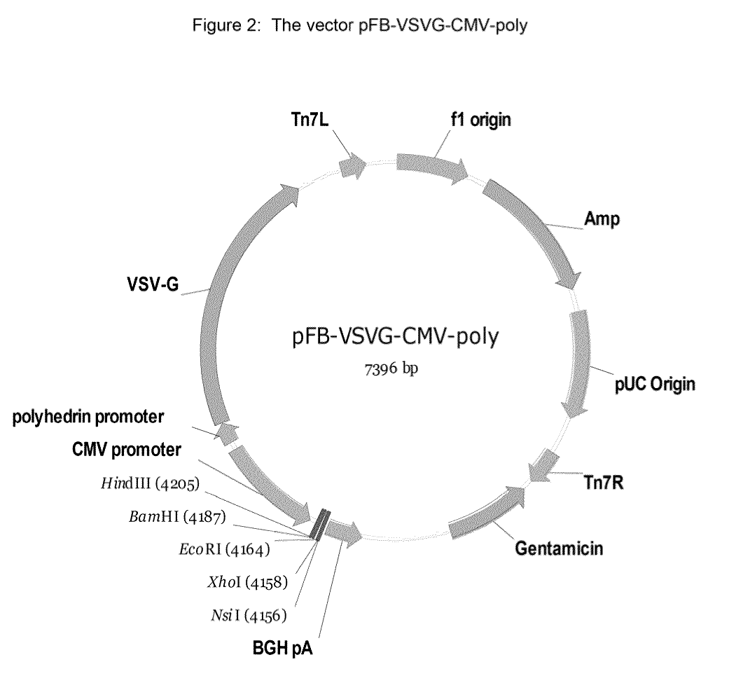

The amplified product was purified (Qiaquick Kit, Qiagen, Valencia, Calif.) and digested with BamH1 and EcoRI (New England BioLabs, Ipswich, Mass.) according to the manufacturer's protocols. The vector pFB-VSVG-CMV-poly (FIG. 2) was digested with BamHI and EcoRI (New England BioLabs, Ipswich, Mass.). The digested DNA was separated by electrophoresis on a 1% agarose gel; the fragments were excised from the gel and purified (Qiaquick Kit, Qiagen, Valencia, Calif.). The vector and gene fragments were ligated (Rapid Ligase Kit, Roche, Pleasanton, Calif.) and transformed into OneShot DH5alpha T1R cells (Invitrogen, Carlsbad, Calif.). Eight ampicillin-resistant colonies (“clones 1-8”) were grown for miniprep (Qiagen Miniprep Kit, Qiagen, Valencia, Calif.) and sequenced to confirm identity and correct insert orientation.

The pFB-VSVG-CMV-poly-hGPR119 construct (clone #1) was transformed into OneShot DH10Bac cells (Invitrogen, Carlsbad, Calif.) according to manufacturers' protocols. Eight positive (i.e. white) colonies were re-streaked to confirm as “positives” and subsequently grown for bacmid isolation. The recombinant hGPR119 bacmid was isolated via a modified Alkaline Lysis procedure using the buffers from a Qiagen Miniprep Kit (Qiagen, Valencia, Calif.). Briefly, pelleted cells were lysed in buffer P1, neutralized in buffer P2, and precipitated with buffer N3. Precipitate was pelleted via centrifugation (17,900×g for 10 minutes) and the supernatant was combined with isopropanol to precipitate the DNA. The DNA was pelleted via centrifugation (17,900×g for 30 minutes), washed once with 70% ethanol, and resuspended in 50 μL buffer EB (Tris-HCL, pH 8.5). Polymerase chain reaction (PCR) with commercially available primers (M13F, M13R, Invitrogen, Carlsbad, Calif.) was used to confirm the presence of the hGPR119 insert in the Bacmid.

Generation of hGPR119 Recombinant Baculovirus

### Creation of P0 Virus Stock

Suspension adapted Sf9 cells grown in Sf900II medium (Invitrogen, Carlsbad, Calif.) were transfected with 10 microL hGPR119 bacmid DNA according to the manufacturer's protocol (Cellfectin, Invitrogen, Carlsbad, Calif.). After five days of incubation, the conditioned medium (i.e. “P0” virus stock) was centrifuged and filtered through a 0.22 μm filter (Steriflip, Millipore, Billerica, Mass.).

### Creation of Frozen Virus (BIIC) Stocks

For long term virus storage and generation of working (i.e. “P1”) viral stocks, frozen BIIC (Baculovirus Infected Insect Cells) stocks were created as follows: suspension adapted Sf9 cells were grown in Sf900II medium (Invitrogen, Carlsbad, Calif.) and infected with hGPR119 P0 virus stock. After 24 hours of growth, the infected cells were gently centrifuged (approximately 100×g), resuspended in Freezing Medium (10% DMSO, 1% Albumin in Sf900II medium) to a final density of 1×10⁷ cells/mL and frozen according to standard freezing protocols in 1 mL aliquots.

### Creation of Working (“P1”) Virus Stock

Suspension adapted Sf9 cells grown in Sf900II medium (Invitrogen, Carlsbad, Calif.) were infected with a 1:100 dilution of a thawed hGPR119 BIIC stock and incubated for several days (27 degrees Celsius with shaking). When the viability of the cells reached 70%, the conditioned medium was harvested by centrifugation and the virus titer determined by ELISA (BaculoElisa Kit, Clontech, Mountain View, Calif.)

Over-Expression of hGPR119 in Suspension-Adapted HEK 293FT Cells

HEK 293FT cells (Invitrogen, Carlsbad, Calif.) were grown in a shake flask in 293Freestyle medium (Invitrogen) supplemented with 50 microg/mL neomycin and 10 mM HEPES (37 C, 8% carbon dioxide, shaking). The cells were centrifuged gently (approximately 500×g, 10 minutes) and the pellet resuspended in a mixture of Dulbecco's PBS (minus Mg++/−Ca++) supplemented with 18% fetal bovine serum (Sigma Aldrich) and P1 virus such that the multiplicity of infection (MOI) was 10 and the final cell density was 1.3×10⁶/mL (total volume 2.5 liters). The cells were transferred to a 5 liter Wave Bioreactor Wavebag (Wave Technologies, MA) and incubated for 4 hours at 27 degrees Celsius (17 rocks/min, 7 degrees platform angle); at the end of the incubation period, an equal volume(2.5 liters) of 293Freestyle medium supplemented with 30 mM sodium butyrate (Sigma Aldrich) was added (final concentration=15 mM), and the cells were grown for 20 hours (37 degrees Celsius, 8% CO2 [0.2 liters/min}, 25 rocks/minute, 7 degrees platform angle). Cells were harvested via centrifugation (3,000×g, 10 minutes), washed once on DPBS (minus Ca++/Mg++), resuspended in 0.25M sucrose, 25 mM HEPES, 0.5 mM EDTA, pH 7.4 and frozen at −80 degrees Celsius.

### Membrane Preparation for Radioligand Binding Assays

The frozen cells were thawed on ice and centrifuged at 700×g (1400 rpm) for 10 minutes at 4 degrees Celsius. The cell pellet was resuspended in 20 mL phosphate-buffered saline, and centrifuged at 1400 rpm for 10 minutes. The cell pellet was then resuspended in homogenization buffer (10 mM HEPES (Gibco #15630), pH 7.5, 1 mM EDTA (BioSolutions, #BIO260-15), 1 mM EGTA (Sigma, #E-4378), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) and incubated on ice for 10 minutes. Cells were then lysed with 15 gentle strokes of a tight-fitting glass Dounce homogenizer. The homogenate was centrifuged at 1000×g (2200 rpm) for 10 minutes at 4 degrees Celsius. The supernatant was transferred into fresh centrifuge tubes on ice. The cell pellet was resuspended in homogenization buffer, and centrifuged again at 1000×g (2200 rpm) for 10 minutes at 4 degrees Celsius after which the supernatant was removed and the pellet resuspended in homogenization buffer. This process was repeated a third time, after which the supernatants were combined, Benzonase (Novagen #71206) and MgCl₂ (Fluka #63020) were added to final concentrations of 1 U/mL and 6 mM, respectively, and incubated on ice for one hour. The solution was then centrifuged at 25,000×g (15000 rpm) for 20 minutes at 4 degrees Celsius, the supernatant was discarded, and the pellet was resuspended in fresh homogenization buffer (minus Benzonase and MgCl₂). After repeating the 25,000×g centrifugation step, the final membrane pellet was resuspended in homogenization buffer and frozen at −80 degrees Celsius. The protein concentration was determined using the Pierce BCA protein assay kit (Pierce reagents A #23223 and B #23224).

### Synthesis and Purification of [³H]-Compound A

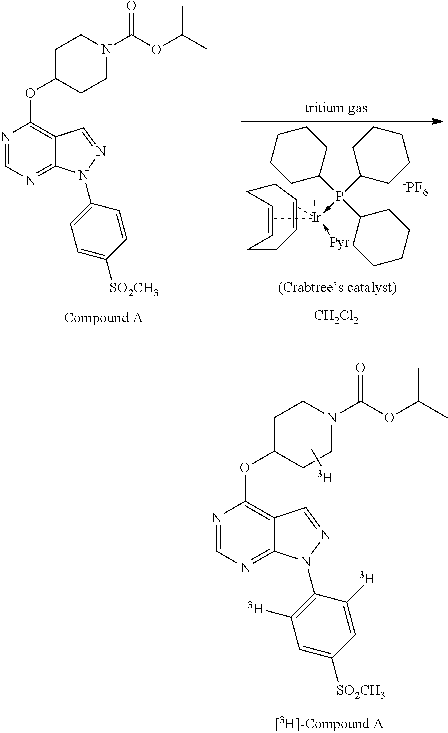

Compound A (isopropyl 4-(1-(4-(methylsulfonyl)phenyl)-3a,7a-dihydro-1H-pyrazolo[3,4-d]pyrimidin-4-yloxy)piperidine-1-carboxylate, as shown above) (4 mg, 0.009 mmol) was dissolved in 0.5 mL of dichloromethane, and the resulting solution was treated with (1,5-cyclooctadiene)(pyridine)(tricyclohexylphosphine)-iridium(1) hexaflurophosphate (J. Organometal. Chem. 1979, 168, 183) (5 mg, 0.006 mmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound A was performed by preparative HPLC using the following conditions.

| Column: | Atlantis, 4.6 × 150 mm, 5 μm | |
|---|---|---|
| Mobil Phase A: | water/acetonitrile/formic acid (98/2/0.1) | |
| Mobil Phase B: | acetonitrile | |
| Gradient: | Time | % B |
| | 0.00 | 30.0 |
| | 1.00 | 30.0 |
| | 13.00 | 80.0 |

| | |
|---|---|
| Run time: | 16 min |
| Post time: | 5 min |
| Flow Rate: | 1.5 mL/minute |
| Inj. Volume: | 20~50 μL |
| Inj. Solvent: | DMSO |
| Detection: | UV at 210 nm and 245 nm |

The specific activity of purified [³H]-Compound A was determined by mass spectroscopy to be 70 Ci/mmol.

Alternatively the binding assay can be performed with [³H]-Compound B.

### Synthesis and Purification of [³H]-Compound B

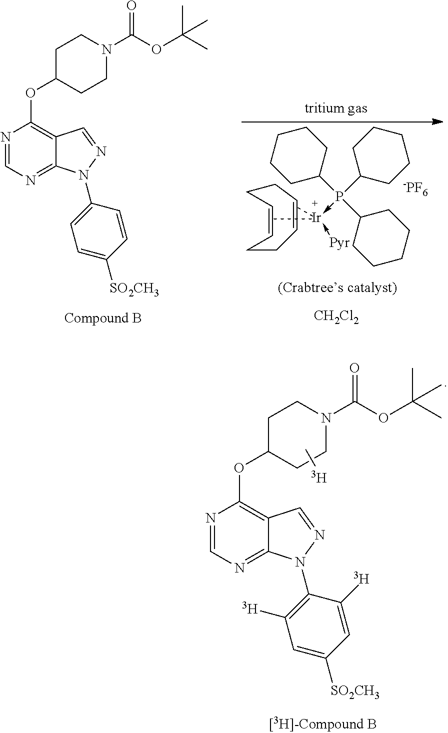

Compound B (tert-butyl 4-(1-(4-(methylsulfonyl)phenyl)-1H-pyrazolo[3,4-d]pyrimidin-4-yloxy)piperidine-1-carboxylate, as shown above) (5 mg, 10.6 μmol) was dissolved in 1.0 mL of dichloromethane and the resulting solution was treated with Crabtree's catalyst (5 mg, 6.2 μmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound B was performed by silica gel flash column chromatography eluting with 70% hexanes/30% ethyl acetate, followed by silica gel flash column chromatography eluting with 60% petroleum ether/40% ethyl acetate.

The specific activity of purified [³H]-Compound B was determined by mass spectroscopy to be 57.8 Ci/mmol.

### GPR119 Radioligand Binding Assay

Test compounds were serially diluted in 100% DMSO (J. T. Baker #922401). 2 microL of each dilution was added to appropriate wells of a 96-well plate (each concentration in triplicate). Unlabeled Compound A (or Compound B), at a final concentration of 10 microM, was used to determine non-specific binding.

[³H]-Compound A (or [³H]-Compound B) was diluted in binding buffer (50 mM Tris-HCl, pH 7.5, (Sigma #T7443), 10 mM MgCl₂ (Fluka 63020), 1 mM EDTA (BioSolutions #BIO260-15), 0.15% bovine serum albumin (Sigma #A7511), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) to a concentration of 60 nM, and 100 microL added to all wells of 96-well plate (Nalge Nunc #267245).

Membranes expressing GPR119 were thawed and diluted to a final concentration of 20 μg/100 microL per well in Binding Buffer, and 100 microL of diluted membranes were added to each well of 96-well plate.

The plate was incubated for 60 minutes w/shaking at room temperature (approximately 25 degrees Celsius). The assay was terminated by vacuum filtration onto GF/C filter plates (Packard #6005174) presoaked in 0.3% polyethylenamine, using a Packard harvester. Filters were then washed six times using washing buffer (50 mM Tris-HCl, pH 7.5 kept at 4 degrees Celsius). The filter plates were then air-dyed at room temperature overnight. 30 μl of scintillation fluid (Ready Safe, Beckman Coulter #141349) was added to each well, plates were sealed, and radioactivity associated with each filter was measured using a Wallac Trilux MicroBeta, plate-based scintillation counter.

The Kd for [³H]-Compound A (or [³H]-Compound B) was determined by carrying out saturation binding, with data analysis by non-linear regression, fit to a one-site hyperbola (Graph Pad Prism). IC₅₀ determinations were made from competition curves, analyzed with a proprietary curve fitting program (SIGHTS) and a 4-parameter logistic dose response equation. Ki values were calculated from IC₅₀ values, using the Cheng-Prusoff equation.

The following results were obtained for the Beta-lactamase and Beta-arrestin functional assays:

| | B- | | | | | |
|---|---|---|---|---|---|---|
| | lactamase | Human B- | | | Human B- | |
| | Functional | lactamase | | B-arrestin | arrestin | Intrinsic |
| | Run | Functional | Intrinsic | Functional | Functional | Activity* |
| Example | Number | EC50 (nM) | Activity* (%) | Run Number | EC50 (nM) | (%) |
| Example 1 | 1 | 68 | 100 | | | |
| | 2 | 122 | 101 | | | |
| Example 2 | 1 | 17 | 93 | | | |
| | 2 | 17 | 84 | | | |
| | 3 | 9 | 100 | | | |
| Example 3 | 1 | 8 | 98 | 1 | 4 | 111 |
| | 2 | 11 | 105 | | | |
| | 3 | 6 | 96 | | | |
| Example 4 | 1 | 15 | 97 | | | |
| | 2 | 16 | 95 | | | |
| Example 5 | 1 | 250 | 100 | | | |
| | 2 | 227 | 88 | | | |
| | 3 | 395 | 104 | | | |
| Example 6 | 1 | 6 | 31 | | | |
| Example 7 | 1 | 622 | 98 | | | |
| Example 8 | 1 | 20 | 100 | 1 | 16 | 80 |
| | 2 | 39 | 102 | | | |
| | 3 | 64 | 94 | | | |
| Example 9 | 1 | 56 | 98 | | | |
| | 2 | 89 | 93 | | | |
| Example 10 | 1 | 34 | 100 | | | |
| | 2 | 38 | 94 | | | |
| Example 11 | 1 | 782 | 98 | | | |
| Example 12 | 1 | 5200 | 100** | | | |
| Example 13 | | | | 1 | 217 | 119 |
| Example 14 | | | | 1 | 495 | 109 |
| Example 15 | | | | 1 | 63 | 104 |
| Example 16 | | | | 1 | 472 | 87 |
| | | | | 2 | 420 | 100** |
| Example 17 | | | | 1 | 161 | 67 |
| Example 19 | 1 | 4000 | 100** | | | |
| | 2 | 4810 | 100** | | | |
| Example 20 | 1 | 61 | 107 | | | |
| | 2 | 131 | 100** | | | |
| Example 21 | 1 | 688 | 100 | | | |
| | 2 | 201 | 96 | | | |
| Example 24 | 1 | 141 | 103 | 1 | 52 | 129 |
| Example 27 | 1 | 1870 | 102 | | | |
| | 2 | 3400 | 100 | | | |
| Exmaple 28 | 1 | 5 | 27 | | | |
| | 2 | 3 | 22 | | | |
| Example 29 | 1 | 3010 | 100** | | | |
| Example 30 | 1 | 136 | 113 | | | |
| | 2 | 166 | 76 | | | |
| Example 31 | | | | 1 | 155 | 92 |
| Example 32 | 1 | 281 | 86 | | | |
| Example 33 | | | | 1 | 1330 | 100** |
| Example 34 | 1 | 270 | 102 | | | |
| Example 35 | 1 | 76 | 83 | | | |
| | 2 | 125 | 101 | | | |
| Example 36 | 1 | 848 | 100 | | | |
| Example 37 | 1 | 328 | 114 | | | |
| | 2 | 805 | 105 | | | |

*The intrinsic activity is the percent of maximal activity of the test compound, relative to the activity of a standard GPR119 agonist, 4-[[6-[(2-fluoro-4 methylsulfonylphenyl)amino]pyrimidin-4-yl]oxy]piperidine-1-carboxylic acid isopropyl ester (WO2005121121), at a final concentration of 10 microM.
**the curve was extrapolated to 100% to calculate an EC50.

The following results were obtained for the cAMP and binding assays:

| | cAMP | Human cAMP | | | Human |
|---|---|---|---|---|---|
| | Functional | Functional | Intrinsic | Binding Run | Binding Ki |
| Example | Run Number | EC50 (nM) | Activity* (%) | Number | (nM) |
| Example 1 | 1 | 217 | 56 | 1 | 47 |
| | 2 | 180 | 37 | 2 | 45 |
| Example 2 | 1 | 29 | 60 | 1 | 19 |
| | 2 | 29 | 63 | 2 | 13 |
| | 3 | 27 | 60 | 3 | 40 |
| | | | | 4 | 10 |
| | | | | 5 | 10 |
| | | | | 6 | 10 |
| Example 3 | 1 | 13 | 86 | 1 | 5 |
| | 2 | 214 | 91 | 2 | 10 |
| | 3 | 239 | 74 | 3 | 37 |
| | 4 | 152 | 79 | | |
| | 5 | 11 | 84 | | |
| Example 4 | 1 | 10 | 80 | 1 | 5 |
| | 2 | 9 | 60 | | |
| Example 5 | 1 | 442 | 39 | 1 | 939 |
| | 2 | 650 | 34 | 2 | 1710 |
| Example 6 | 1 | >10000 | 17 | 1 | >6100 |
| | | | | 2 | >6100 |
| Example 7 | 1 | 225 | 80 | 1 | 293 |
| | 2 | 256 | 74 | 2 | 283 |
| | 3 | 169 | 85 | | |
| Example 8 | 1 | 9 | 89 | 1 | 1 |
| | 2 | 6 | 75 | 2 | 58 |
| | 3 | 7 | 74 | 3 | 43 |
| | | | | 4 | 47 |
| | | | | 5 | 14 |
| Example 9 | | | | 1 | 35 |
| | | | | 2 | 42 |
| Example 10 | 1 | 87 | 62 | 1 | 28 |
| | 2 | 98 | 57 | 2 | 30 |
| Example 11 | 1 | 163 | 61 | 1 | 530 |
| | 2 | 98 | 74 | | |
| | 3 | 154 | 62 | | |
| Example 12 | 1 | >10000 | 31 | 1 | 1700 |
| Example 13 | 1 | 180 | 95 | 1 | 149 |
| | 2 | 112 | 87 | | |
| Example 14 | 1 | 436 | 74 | 1 | 723 |
| | 2 | 494 | 78 | | |
| Example 15 | 1 | 198 | 102 | 1 | 268 |
| | 2 | 106 | 112 | 2 | 160 |
| | 3 | 129 | 131 | | |
| Example 16 | 1 | 204 | 105 | | |
| | 2 | 165 | 102 | | |
| | 3 | 316 | 119 | | |
| Example 17 | 1 | 39 | 95 | | |
| | 2 | 26 | 91 | | |
| Example 18 | 1 | 67 | 80 | 1 | 153 |
| | 2 | 69 | 75 | | |
| Example 19 | 1 | 4480 | 100** | 1 | >6100 |
| | 2 | 4550 | 99 | 2 | >6100 |
| Example 20 | 1 | 59 | 123 | 1 | 41 |
| | 2 | 90 | 107 | 2 | 144 |
| Example 21 | 1 | 44 | 33 | 1 | 58 |
| | 2 | 32 | 31 | 2 | 128 |
| | 3 | 57 | 34 | | |
| Example 22 | 1 | 147 | 29 | 1 | 159 |
| | 2 | 161 | 29 | | |
| Example 23 | 1 | 1640 | 100** | 1 | 150 |
| | 2 | 1470 | 100** | | |
| Example 24 | 1 | 27 | 111 | 1 | 54 |
| | 2 | 29 | 100 | | |
| Example 25 | 1 | 57 | 88 | 1 | 418 |
| | 2 | 111 | 114 | | |
| Example 26 | 1 | 254 | 62 | | |
| Example 27 | 1 | 394 | 27 | 1 | 1640 |
| | | | | 2 | 528 |
| Example 28 | 1 | >10000 | | 1 | >6100 |
| | | | | 2 | >6100 |
| Example 29 | 1 | >10000 | 27 | 1 | 1550 |
| | 2 | 648 | 33 | 2 | 3380 |
| Example 30 | 1 | 256 | 68 | 1 | 2020 |
| | 2 | 245 | 60 | | |
| Example 31 | 1 | 56 | 107 | 1 | 462 |
| | 2 | 52 | 109 | | |
| Example 32 | 1 | 66 | 66 | 1 | 514 |
| | 2 | 64 | 78 | | |
| | 3 | 58 | 60 | | |
| Example 33 | 1 | 244 | 85 | | |
| | 2 | 274 | 92 | | |
| Example 34 | 1 | 55 | 94 | 1 | 87 |
| | 2 | 70 | 78 | | |
| Example 35 | 1 | 18 | 76 | 1 | 31 |
| | 2 | 31 | 66 | 2 | 29 |
| Example 36 | 1 | 281 | 51 | 1 | 521 |
| | 2 | 256 | 76 | 2 | 1450 |
| Example 37 | 1 | 1160 | 56 | 1 | 416 |

*The intrinsic activity is the percent of maximal activity of the test compound, relative to the activity of a standard GPR119 agonist, 4-[[6-[(2-fluoro-4 methylsulfonylphenyl)amino]pyrimidin-4-yl]oxy]piperidine-1-carboxylic acid isopropyl ester (WO2005121121), at a final concentration of 10 μM.
**the curve was extrapolated to 100% to calculate an EC50.

### Preparation of Starting Materials

### Preparation 1: Isopropyl 4-hydrazinopiperidine-1-carboxylate dihydrochloride salt

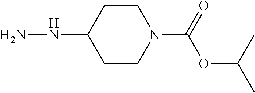

Isopropyl 4-{2-(tert-butoxycarbonyl)hydrazinyl}piperidine-1-carboxylate (obtained as described in WO2008137436) (20.2 g, 67.02 mmol), was dissolved in absolute ethanol (250 mL), and the solution was stirred under nitrogen at room temperature. Concentrated aqueous hydrochloric acid (27.9 mL, 335 mmol) was added slowly. The solution was stirred under nitrogen at room temperature for 4 hours. The reaction was concentrated to a white solid that contained some starting material. The solid was treated with a 4 M solution of hydrogen chloride in 1,4-dioxane (100 mL, 400 mmol) and the resulting mixture was stirred for 14 hours at room temperature. The reaction was then concentrated under reduced pressure to give a white solid, which was treated with heptane (100 mL) and concentrated again to yield the title compound as a white solid (15 g, 81%). ¹H NMR (400 MHz, methanol-d₄) delta 4.9 (m, 1H), 4.1 (m, 2H), 3.2 (m, 1H), 2.9 (m, 2H), 2.0 (m, 2H), 1.4 (m, 2H), 1.2, (d, 6H); LCMS (ES+): 202 (M+1).

### Preparation 2: Isopropyl 4-[5-amino-4-(ethoxycarbonyl)-1H-pyrazol-1-yl]-piperidine-1-carboxylate

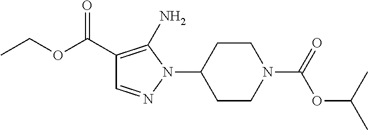

A mixture of isopropyl 4-hydrazinopiperidine-1-carboxylate dihydrochloride salt (7.08 g, 25.8 mmol), ethyl 2-cyano-3-ethoxyacrylate (4.81 g, 28.4 mmol), sodium acetate (6.49 g, 77.5 mmol), and ethanol (80 mL) was stirred at 85° C. for 3 hours. The mixture was concentrated to about a third of the initial volume. Water (50 mL), saturated sodium bicarbonate (50 mL), and brine (50 mL) were added. The resulting mixture was extracted with ethyl acetate (2×50 mL). The combined organic extracts were washed with brine and dried over magnesium sulfate. The mixture was filtered, and the filtrate concentrated under vacuum to obtain the crude title compound as a light yellow solid (9.8 g), which was used in the next step without purification. An analytical sample was prepared by purification via chromatography on silica gel, eluting with a 30% to 60% solution of ethyl acetate in heptane. ¹H NMR (500 MHz, deuterochloroform) delta 1.26 (d, 6H) 1.35 (t, 3H) 1.86-1.95 (m, 2H) 2.04-2.17 (m, 2H) 2.84-2.96 (m, 2H) 3.89-3.98 (m, 1H) 4.28 (q, 2H). 4.25-4.40 (m, 2H) 4.89-4.97 (m, 1H) 5.06 (s, 2H) 7.64 (s, 1H); LCMS (ES+): 325.1 (M+1).

### Preparation 3: Isopropyl 4-[5-bromo-4-(ethoxycarbonyl)-1H-pyrazol-1-yl]piperidine-1-carboxylate

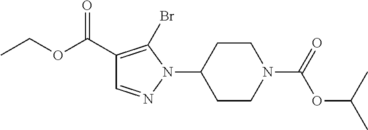

Neat tent-butyl nitrite (4.8 mL, 39.3 mmol) was added slowly to a stirred mixture of isopropyl 4-[5-amino-4-(ethoxycarbonyl)-1H-pyrazol-1-yl]-piperidine-1-carboxylate (Preparation 2) (8.5 g, 26.2 mmol) and copper (II) bromide (3.7 g, 16 mmol) in acetonitrile (100 mL) at room temperature. A significant exothermic effect was observed with the mixture warming to about 50° C. After continued heating at 65° C. for 30 minutes, the reaction was cooled to room temperature, and then concentrated under vacuum. An excess of 10% aqueous ammonia was added, and the mixture was extracted with ethyl acetate. The organic phase was washed with water and brine, and concentrated under vacuum. The residue was purified by chromatography on silica gel eluting with 30% to 70% ethyl acetate in heptane to provide the title compound as a yellow oil, which was about 70% pure by NMR and LCMS. The material was used in the next step without further purification. ¹H NMR (400 MHz, deuterochloroform) delta 1.23 (d, 6H) 1.34 (t, 3H) 1.84-1.95 (m, 2H) 2.01-2.15 (m, 2H) 2.82-2.98 (m, 2H) 4.25-4.36 (m, 2H) 4.30 (q, 2H) 4.45-4.56 (m, 1H) 4.86-4.96 (m, 1H) 7.95 (s, 1H); LCMS (ES+): 387.9 (M+1).

### Preparation 4: Isopropyl 4-[5-bromo-4-(hydroxymethyl)-1H-pyrazol-1-yl]piperidine-1-carboxylate

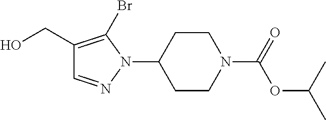

To a solution of isopropyl 4-[5-bromo-4-(ethoxycarbonyl)-1H-pyrazol-1-yl]piperidine-1-carboxylate (3.59 g, 6.5 mmol) in tetrahydrofuran (32 mL) cooled to 0° C. was added a 2 M solution of borane-methyl sulfide complex in tetrahydrofuran (14.6 mL, 29.2 mmol). The reaction mixture was heated at reflux for 21 hours and then stirred for 4 hours at room temperature. The mixture was cooled to 0° C., and methanol was added. The resulting solution was warmed to room temperature and stirred for 10 minutes. The solution was re-cooled to 0° C. and aqueous 2 M sodium hydroxide solution (10 mL) was added dropwise. The resulting mixture was diluted with ethyl acetate and stirred vigorously for 30 minutes. The layers were separated, and the aqueous phase was extracted twice with ethyl acetate. The combined organic layers were washed sequentially with water and brine and then dried over magnesium sulfate. The mixture was filtered, and the filtrate concentrated under vacuum. Chromatography over silica gel eluting with 55% to 70% ethyl acetate in heptane gave the title compound as an oil (1.89 g, 84%). ¹H NMR (400 MHz, deuterochloroform) delta 1.23 (d, 6H), 1.87-1.95 (br m, 3H), 2.06 (qd, 2H), 2.89 (br t, 2H), 4.29 (br s, 2H), 4.39 (tt, 1H), 4.50 (d, 2H), 4.90 (m, 1H), 7.58 (s, 1H); LCMS (ES+) 348.0 (M+1).

### Preparation 5: Isopropyl 4-[5-cyano-4-(hydroxymethyl)-1H-pyrazol-1-yl]piperidine-1-carboxylate

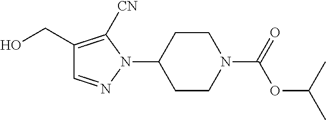

Isopropyl 4-[5-bromo-4-(hydroxymethyl)-1H-pyrazol-1-yl]piperidine-1-carboxylate (1.42 g, 4.10 mmol), tris-(dibenzylideneacetone)dipalladium (156 mg, 0.170 mmol), 1-1′-bis-(diphenylphosphino)ferrocene (192 mg, 0.346 mmol), zinc dust (68.8 mg, 1.06 mmol), zinc cyanide (497 mg, 4.23 mmol) and N,N-dimethylacetamide (20 mL) were combined in a microwave vial. The vial was flushed with nitrogen, sealed and heated at 120° C. for 1 hour in a microwave reactor (Biotage Initiator 2.2). The reaction mixture was passed through a pad of Florisil™, diluted with ethyl acetate and then water was added. The aqueous phase was extracted 3 times with ethyl acetate and the combined organic layers were dried over magnesium sulfate. The mixture was filtered, and the filtrate evaporated under vacuum. Chromatography on silica gel eluting with 55% to 70% ethyl acetate in heptane gave the title compound as a green oil that solidified upon standing (1.06 g, 88%). ¹H NMR (400 MHz, deuterochloroform) delta 1.24 (d, 6H), 1.99 (br d, 2H), 2.06-2.17 (m, 3H), 2.93 (br t, 2H), 4.31 (br s, 2H), 4.48 (tt, 1H), 4.71 (d, 2H), 4.92 (m, 1H), 7.60 (s, 1H); LCMS (ES+): 293.1 (M+H).

### Preparation 6: 2-Fluoro-4-[(2-hydroxyethyl)thio]phenol

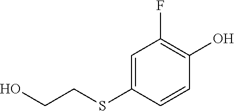

To a solution of 4-bromo-2-fluorophenol (0.75 mL, 6.8 mmol) and diisopropylethylamine (3.5 mL, 20.09 mmol) in 1,4-dioxane (35 mL) was added 9,9-dimethyl-4,5-bis(diphenylphosphino)xanthene (415 mg, 0.717 mmol), bis(dibenzylideneacetone)palladium (322 mg, 0.351 mmol) and 2-mercaptoethanol (0.46 mL, 6.86 mmol), and the dark brown reaction solution was heated at 110° C. for 16 hours. The reaction was allowed to cool to room temperature, diluted with water and extracted with ethyl acetate four times. The organic extracts were combined and dried over magnesium sulfate, The mixture was filtered, and the filtrate concentrated under reduced pressure to give a maroon oil which was purified by chromatography on silicon gel to afford the title compound (985 mg, 76%) as a maroon solid. ¹H NMR (400 MHz, deuterochloroform) delta 3.00 (t, 2H, J=5.95 Hz) 3.69 (d, 2H, J=3.71 Hz) 6.89-6.95 (m, 1H) 7.11 (ddd, 1H, J=8.39, 2.15, 1.17 Hz) 7.17 (dd, 1H, J=10.54, 2.15 Hz).

### Preparation 7: 4-[(2-{[tert-Butyl(dimethyl)silyl]oxy}ethyl)thio]-2-fluorophenol

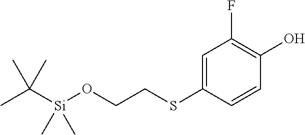

To a solution of 2-fluoro-4-[(2-hydroxyethyl)thio]phenol (985 mg, 5.24 mmol) and imidazole (371 mg, 5.30 mmol) in N,N-dimethylformamide (5 mL) was added tert-butyldimethylsilyl chloride (814 mg, 5.24 mmol) portion-wise, and the reaction was stirred at room temperature for 4 hours. The reaction was concentrated under reduced pressure, and the residue diluted with water followed by extraction with ethyl acetate three times. The combined organic extracts were washed with brine and dried over magnesium sulfate. The mixture was filtered, and the filtrate concentrated under reduced pressure to give the title compound as an orange oil (1.43 g, 90%) which was used without further purification. LCMS (ES+): 301.1 (M−1).

### Preparation 8: 1-[4-(Benzyloxy)-3-fluorophenyl]-1H-tetrazole

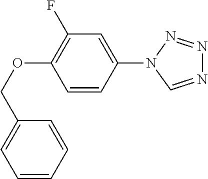

To a suspension of 4-(benzyloxy)-3-fluoroaniline (1.04 g, 4.8 mmol) (WO 2005030140) under a nitrogen atmosphere was added acetic acid (2.3 mL, 38.3 mmol), triethy lorthoformate (2.44 mL, 14.4 mmol) and sodium azide (0.34 g, 5.3 mmol), and the reaction mixture heated at 95° C. for 2.5 hours. The solution was then allowed to cool to room temperature, and water was added followed by extraction with ethyl acetate three times. The extracts were combined and washed with brine and dried over magnesium sulfate. The mixture was filtered and concentrated under reduced pressure, and the crude material purified by chromatography on silicon gel (20-40% ethyl acetate in heptane) to give the title compound as a white solid (1.12 g, 86%). ¹H NMR (400 MHz, deuteromethanol) delta 9.65 (s, 1H), 7.73-7.68 (dd, 1H, J=11, 2.5 Hz), 7.60-7.57 (m, 1H) 7.47-7.45 (m, 2H), 7.40-7.30 (m, 5H), 5.24 (s, 2H); LCMS (ES+): 271.1 (M+1).

### Preparation 9: 2-Fluoro-4-(1H-tetrazol-1-yl)phenol

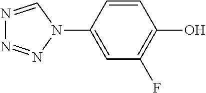

To 1-[4-(benzyloxy)-3-fluorophenyl]-1H-tetrazole (1.12 g, 4.14 mmol) in a Parr shaker flask was added ethanol (40 mL), and the solution purged with nitrogen gas. 10% palladium on carbon (0.30 g) was added, and the reaction hydrogenated on a Parr shaker apparatus at 40 psi of hydrogen for 30 minutes. The mixture was then filtered through a micro pore filter, and the filtrate was concentrated under reduced pressure to yield the title compound as a white solid (0.67 g, 90%) which was use without purification. ¹H NMR (400 MHz, deuteromethanol) delta 9.62 (s, 1H), 7.65-7.62 (dd, 1H, J=11, 2.5 Hz), 7.50-7.46 (m, 1H) 7.47-7.45 (dd, 1H, J=9.0, 9.0 Hz); LCMS (ES+): 181.1 (M+1).

### Preparation 10: Isopropyl 4-(5-cyano-4-((methylsulfonyloxy)methyl)-1H-pyrazol-1-yl)piperidine-1-carboxylate

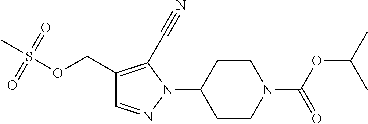

Isopropyl 4-(5-cyano-4-(hydroxymethyl)-1H-pyrazol-1-yl)piperidine-1-carboxylate (Preparation 5) (75 mg, 0.24 mmol) was dissolved in 1 mL of anhydrous dichloromethane and triethylamine (0.1 mL, 0.74 mmol) was added. The reaction mixture was cooled in an ice bath and methanesulfonic anhydride (62 mg, 0.34 mmol) was then added. The solution was removed from the ice bath and stirred for 30 minutes. The reaction was quenched by addition of saturated aqueous sodium bicarbonate and the layers were separated. The aqueous layer was extracted three more times with dichloromethane. The organic extracts were combined and washed with brine, dried over sodium sulfate, filtered and the filtrate was concentrated to give an oil (75 mg, 100% yield). The crude material was used in subsequent steps without further purification.

### Preparation 11: tert-Butyl 4-hydrazinopiperidine-1-carboxylate hydrochloride salt

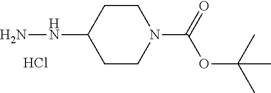

Into a solution of tert-butyl 4-oxopiperidine-1-carboxylate (50 g, 0.25 mmol) in methanol (500 mL) in an autoclave was added hydrazine mono-hydrochloride (17.2 g, 0.25 mmol) in water (100 mL). The white mixture was stirred under argon followed by the addition of 5% platinum on carbon (750 mg) as a slurry in water. The autoclave was sealed and charged to 60 atmospheres with hydrogen, and the reaction was stirred for 15 hours. Upon completion, the reaction was filtered through Celite®, and the pad washed with methanol. This preparation was carried out six times. The combined filtrates were concentrated under reduced pressure, and the resulting white precipitate (di-tert-butyl-4,4′-hydrazine-1,2-diyldipiperidine-1-carboxylate) by-product was collected by filtration and washed several times with water. The aqueous filtrate was then concentrated under reduced pressure to give the desired product (221 g, 59%) as a colorless solid. ¹H NMR (400 MHz, deuterochloroform) delta 4.13 (br s, 2H), 3.32 (br t, 1H), 2.77 (br t, 2H), 2.16 (m, 2H), 1.66 (m, 2H), 1.43 (s, 9H).

### Preparation 12: tert-Butyl 4-[5-amino-4-(ethoxycarbonyl)-1H-pyrazol-1-yl]piperidine-1-carboxylate

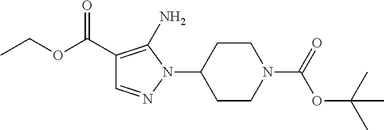

A mixture of tert-butyl 4-hydrazinopiperidine-1-carboxylate hydrochloride salt (221 g, 880 mmol), ethyl 2-cyano-3-ethoxyacrylate (153 g, 880 mmol), sodium acetate trihydrate (477 g, 352 mmol) and ethanol (2000 mL) was stirred at 85 degrees Celsius for 8 hours. The mixture was concentrated under reduced pressure, and the residue dissolved in ethyl acetate and water. The layers were separated, and the aqueous layer extracted with ethyl acetate. The combined organic extracts were then dried over magnesium sulfate. The mixture was filtered, and the filtrate concentrated under reduced pressure. The crude material was purified by filtration through a short plug of silica gel eluting with 40% ethyl acetate in heptane to produce the product as a pale yellow solid (214 g, 72%). ¹H NMR (500 MHz, deuterochloroform) delta 7.60 (s, 1H), 5.27 (br s, 2H), 4.23 (br q, 4H), 3.91 (m, 1H), 2.81 (br s, 2H), 2.04 (m, 2H), 1.86 (m, 2H), 1.44 (s, 9H), 1.29 (t, 3H).

### Preparation 13: tert-Butyl 4-[5-bromo-4-(ethoxycarbonyl)-1H-pyrazol-1-yl]piperidine-1-carboxylate

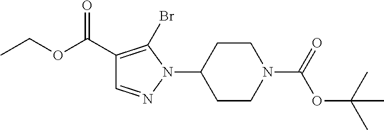

To a solution of copper (II) bromide (1.69 g, 770 mmol) in acetonitrile (1000 mL) was slowly added tert-butyl nitrite (112 mL, 960 mmol), and the solution was heated to 65 degrees Celsius. To this was added a solution of tert-butyl 4-[5-amino-4-(ethoxycarbonyl)-1H-pyrazol-1-yl]piperidine-1-carboxylate (215 g, 640 mmol) in acetonitrile (650 mL) drop-wise over 30 minutes. After 4 hours, the reaction was allowed to cool to room temperature and was then poured into 2 M hydrochloric acid (1500 mL) in ice. The mixture was extracted with ethyl acetate three times, and the combined organic extracts were washed with saturated aqueous sodium bicarbonate and then dried over magnesium sulfate. The mixture was filtered, and the filtrate concentrated under reduced pressure. The resulting residue was purified by filtration through a short plug of silica gel eluting initially with 10% heptane in dichloromethane followed by dichloromethane to give the title compound (137 g, 53%) as a yellow oil which solidified on standing. ¹H NMR (400 MHz, deuterochloroform) delta 7.95 (s, 1H), 4.48 (m, 1H), 4.28 (br q, 4H), 2.86 (br s, 2H), 2.06 (m, 2H), 1.90 (m, 2H), 1.44 (s, 9H), 1.34 (t, 3H).

### Preparation 14: tert-Butyl 4-[5-bromo-4-(hydroxymethyl)-1H-pyrazol-1-yl]piperidine-1-carboxylate

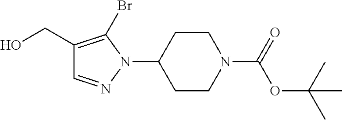

To a solution of tert-butyl 4-[5-bromo-4-(ethoxycarbonyl)-1H-pyrazol-1-yl]piperidine-1-carboxylate (137 g, 0.34 mol) in tetrahydrofuran (1300 mL) cooled to 0 degrees Celsius was slowly added borane-methyl sulfide (97 mL, 1.02 mol). The solution was allowed to warm to room temperature and then heated at reflux for 15 hours. The reaction was then cooled in an ice bath, and methanol (40 mL) added drop-wise. The solution was then stirred at room temperature for 20 minutes. Aqueous 2 M sodium hydroxide (1200 mL) was added, and the layers were separated. The aqueous layer was extracted with ethyl acetate, and the combined organics layers were washed with brine, dried over magnesium sulfate, and the solvent removed under reduced pressure. The resulting residue was purified by filtration through a short plug of silica gel eluting with 30% ethyl acetate in heptane to reveal the title compound as an colorless solid (61.4 g, 50%). Impure material from this purification was further purified via the above chromatographic procedure to provide a second batch of the title compound (22 g, 18%) as a colorless solid. ¹H NMR (400 MHz, deuterochloroform) delta 7.59 (s, 1H), 4.52 (s, 2H), 4.37 (m, 1H), 4.25 (br s, 2H), 2.86 (br s, 2H), 2.06 (m br s, 2H), 1.89 (m, 2H), 1.45 (s, 9H).

### Preparation 15: tert-Butyl 4-[5-cyano-4-(hydroxymethyl)-1H-pyrazol-1-yl]piperidine-1-carboxylate

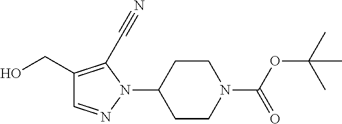

Copper (I) cyanide (2.97 g, 33.3 mmol) was added to a stirred solution of tert-butyl 4-[5-bromo-4-(hydroxymethyl)-1H-pyrazol-1-yl]piperidine-1-carboxylate (10 g, 27.8 mmol) in degassed dimethylformamide (100 mL). The reaction was then heated at 165 degrees Celsius for 4 hours and allowed to cool to room temperature. It was further cooled in an ice-bath, and a solution of ethylenediamine (5.5 mL) in water (20 mL) was added followed by dilution with more water (70 mL). The mixture was then extracted with ethyl acetate, and the layers separated. The organic layer was washed sequentially with water and brine and then dried over magnesium sulfate. The mixture was filtered and the filtrate concentrated under reduced pressure. This procedure was carried out in 8 batches. The final crude residues were combined and purified by repeated silica gel column chromatography eluting with 40% ethyl acetate in heptane to give the title compound (11.6 g, 17%) as a colorless solid. ¹H NMR (400 MHz, deuterochloroform) 7.59 (s, 1H), 4.71 (s, 2H), 4.45 (m, 1H), 4.26 (br s, 2H), 2.88 (br t, 2H), 2.08 (m, 2H), 1.98 (m, 2H), 1.48 (s, 9H); LCMS (ES+): 207.1 (M-Boc+H).

### Preparation 16: tert-Butyl 4-(5-cyano-4-((methylsulfonyloxy)methyl)-1H-pyrazol-1-yl)piperidine-1-carboxylate

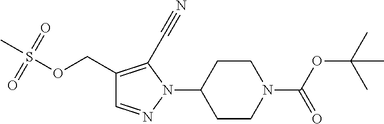

To a stirred solution of tert-butyl 4-(5-cyano-4-(hydroxymethyl)-1H-pyrazol-1-yl)piperidine-1-carboxylate (202 mg, 0.659 mmol) in dichloromethane (6.6 mL) was added triethylamine (0.18 mL, 1.32 mmol) followed by methanesulfonic anhydride (189 mg, 1.1 mmol) at room temperature. The mixture was stirred for 4.5 hours before it was diluted with dichloromethane and saturated aqueous bicarbonate. The layers were separated and the aqueous layer was extracted with dichloromethane. The combined organic extracts were washed with brine, dried over magnesium sulfate, filtered and the filtrate was concentrated in vacuo to give tent-butyl 4-(5-cyano-4-((methylsulfonyloxy)methyl)-1H-pyrazol-1-yl)piperidine-1-carboxylate as an oil which was used without further purification.

### Preparation 17: 2-Fluoro-4-(1-((2-(trimethylsilyl)ethoxy)methyl)-1H-tetrazol-5-yl)phenol and 2-Fluoro-4-(2-((2-(trimethylsilyl)ethoxy)methyl)-2H-tetrazol-5-yl)phenol

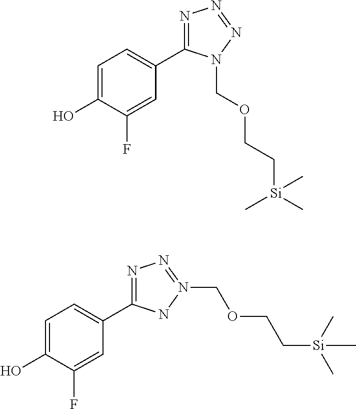

### A) 4-(Benzyloxy)-3-fluorobenzonitrile

To a stirred solution of 3-fluoro-4-hydroxybenzonitrile (1.00 g, 7.30 mmol) in 20 mL of acetonitrile was added portion-wise potassium carbonate (2.02 g, 14.6 mmol). The resulting mixture was stirred for 10 minutes before benzyl bromide (1.33 mL, 10.9 mmol) was added. The mixture was stirred at room temperature for 70 hours before it was diluted with ethyl acetate and water. The organic phase was separated and washed with water, brine, dried over magnesium sulfate, filtered and the filtrate was concentrated in vacuo. The residue was purified by flash chromatography, eluting with a gradient of 5 to 20% of ethyl acetate in heptane to give 4-(benzyloxy)-3-fluorobenzonitrile as a white solid (1.33 g).

### B) 5-(4-(Benzyloxy)-3-fluorophenyl)-1H-tetrazole and 5-(4-(Benzyloxy)-3-fluorophenyl)-2H-tetrazole

A vial charged with 4-(benzyloxy)-3-fluorobenzonitrile (250 mg, 1.10 mmol), sodium azide (214 mg, 3.30 mmol), ammonium chloride (176 mg, 3.30 mmol) and 3 mL of N,N-dimethylformamide was heated at 110 degrees Celsius for 18 hours. The reaction mixture was cooled to room temperature, diluted with water and ethyl acetate and the pH was adjusted to 3 using aqueous 1 N hydrochloric acid. The organic phase was separated and washed with brine, dried over magnesium sulfate, filtered and the filtrate was concentrated in vacuo to give the title compounds as a white solid (270 mg). This material was used in subsequent steps without purification.

### C) 5-(4-(Benzyloxy)-3-fluorophenyl)-1-((2-(trimethylsilyl)ethoxy)methyl)-1H-tetrazole and 5-(4-(Benzyloxy)-3-fluorophenyl)-2-((2-(trimethylsilyl)ethoxy)methyl)-2H-tetrazole

To a solution of 5-(4-(benzyloxy)-3-fluorophenyl)-1H-tetrazole and 5-(4-(benzyloxy)-3-fluorophenyl)-2H-tetrazole (270 mg, 1 mmol) dissolved in tetrahydrofuran was added sodium hydride (44 mg, 1.1 mmol) in four portions and the resulting mixture was stirred at room temperature for 15 minutes. (2-(Chloromethoxy)ethyl)trimethylsilane (0.19 mL, 1.0 mmol) was then added and the reaction mixture was stirred at room temperature for 16 hours. The reaction was quenched by the addition of water and ethyl acetate was added. The organic phase was separated and the aqueous phase was extracted twice with ethyl acetate. The combined organic extracts were washed with brine, dried over magnesium sulfate, filtered and the filtrate was concentrated under reduced pressure. Purification by flash chromatography, eluting with a gradient of ethyl acetate and heptane (5 to 20% ethyl acetate) gave the desired product as a white solid (270 mg, 67% yield).

### D) 2-Fluoro-4-(1-((2-(trimethylsilyl)ethoxy)methyl)-1H-tetrazol-5-yl)phenol and 2-Fluoro-4-(2-((2-(trimethylsilyl)ethoxy)methyl)-2H-tetrazol-5-yl)phenol

To a solution of 5-(4-(benzyloxy)-3-fluorophenyl)-1-((2-(trimethylsilyl)ethoxy)methyl)-1H-tetrazole and 5-(4-(benzyloxy)-3-fluorophenyl)-2-((2-(trimethylsilyl)ethoxy)methyl)-2H-tetrazole (140 mg, 0.35 mmol) dissolved in a mixture of 2 mL of ethanol and 2 mL of tetrahydrofuran was added palladium black (56 mg, 0.53 mmol) and formic acid (0.14 mL, 3.5 mmol). The resulting mixture was stirred at room temperature for 4 hours before being filtered though a pad of Celite®. The filtrate was concentrated under reduced pressure and the resulting crude material was used in the subsequent step without further purification.

### Preparation 18: 5-(4-(Benzyloxy)-3-fluorophenyl)-1-(2-(trimethylsilyloxy)ethyl)-1H-tetrazole and 5-(4-(Benzyloxy)-3-fluorophenyl)-2-(2-(trimethylsilyloxy)ethyl)-2H-tetrazole

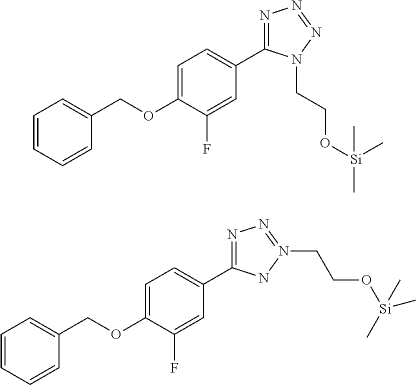

To a solution of 5-(4-(benzyloxy)-3-fluorophenyl)-1H-tetrazole and 5-(4-(Benzyloxy)-3-fluorophenyl)-2H-tetrazole (Preparation 17, Step B) (550 mg, 2 mmol) dissolved in N,N-dimethylformamide (8 mL) was added sodium hydride (163 mg, 4 mmol) in two portions and the resulting mixture was stirred at room temperature for 5 minutes. (2-Bromoethoxy)trimethylsilane (1.3 mL, 6 mmol) was then added and the reaction mixture was stirred at 70 degrees Celsius for 16 hours before being cooled to room temperature. The reaction was quenched by addition of water and ethyl acetate was added. The organic phase was separated and the aqueous phase was extracted twice with ethyl acetate. The combined organic extracts were washed with brine, dried over magnesium sulfate, filtered and concentrated under reduced pressure. The residue was purified by flash chromatography, eluting with a gradient of ethyl acetate and heptane (5 to 30% ethyl acetate) to give 5-(4-(benzyloxy)-3-fluorophenyl)-1-(2-(trimethylsilyloxy)ethyl)-1H-tetrazole (100 mg, 12% yield) and 5-(4-(benzyloxy)-3-fluorophenyl)-2-(2-(trimethylsilyloxy)ethyl)-2H-tetrazole (600 mg, 69% yield).

### Preparation 19: 2-Fluoro-4-(2-(2-(trimethylsilyloxy)ethyl)-2H-tetrazol-5-yl)phenol

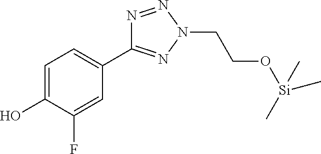

To a solution of 5-(4-(benzyloxy)-3-fluorophenyl)-2-(2-(trimethylsilyloxy)ethyl)-2H-tetrazole (Preparation 18) (230 mg, 0.54 mmol) dissolved in a mixture of 6 mL of ethanol and 6 mL of tetrahydrofuran was added palladium black (86 mg, 0.806 mmol) and formic acid (0.215 mL, 5.4 mmol). The resulting mixture was stirred at room temperature for 4 hours before being filtered through a pad of Celite®. The filtrate was concentrated under reduced pressure and the resulting crude material (180 mg) was used in the subsequent step without further purification.

### Preparation 20: 2-Fluoro-4-(1-(2-(trimethylsilyloxy)ethyl)-1H-tetrazol-5-yl)phenol

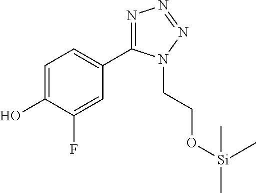

To a solution of 5-(4-(benzyloxy)-3-fluorophenyl)-1-(2-(trimethylsilyloxy)ethyl)-1H-tetrazole (Preparation 18) (130 mg, 0.30 mmol) dissolved in a mixture of 2 mL of ethanol and 2 mL of tetrahydrofuran was added palladium black (48 mg, 0.45 mmol) and formic acid (0.12 mL, 3 mmol). The resulting mixture was stirred at room temperature for 4 hours before being filtered over a pad of Celite®. The filtrate was concentrated under reduced pressure and the resulting crude material (94 mg) was used in the subsequent step without further purification.

### Preparation 21: 2-Fluoro-4-(1-methyl-1H-tetrazol-5-yl)phenol

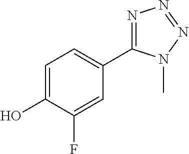

### A) 5-(4-(Benzyloxy)-3-fluorophenyl)-1-methyl-1H-tetrazole and 5-(4-(Benzyloxy)-3-fluorophenyl)-2-methyl-2H-tetrazole

To a stirred solution of 5-(4-(benzyloxy)-3-fluorophenyl)-1H-tetrazole and 5-(4-(benzyloxy)-3-fluorophenyl)-2H-tetrazole (Preparation 17, Step B) (1.50 g, 5.55 mmol) in 30 mL of tetrahydrofuran was added sodium hydride (444 mg, 11.1 mmol) in two portions at room temperature. After 5 minutes, iodomethane (1.04 mL, 16.6 mmol) was added and the reaction was stirred under a nitrogen atmosphere for 15 hours at room temperature. The mixture was diluted with water and extracted twice with ethyl acetate. The combined organic extracts were washed with brine, dried with magnesium sulfate, filtered and the filtrate was concentrated in vacuo. The residue was purified by flash chromatography, eluting with 10-40% ethyl acetate in heptane to give 5-(4-(benzyloxy)-3-fluorophenyl)-2-methyl-2H-tetrazole as a white solid (1.1 g) and 5-(4-(benzyloxy)-3-fluorophenyl)-1-methyl-1H-tetrazole as a white solid (450 mg).

5-(4-(Benzyloxy)-3-fluorophenyl)-1-methyl-1H-tetrazole. ¹H NMR (400 MHz, deuterochloroform) delta 4.15 (s, 3H) 5.23 (s, 2H) 7.15 (t, J=8.39 Hz, 1H) 7.31-7.48 (m, 6H) 7.52 (dd, J=11.13, 2.15 Hz, 1H). LCMS (M+1) 285.1.

### B) 2-Fluoro-4-(1-methyl-1H-tetrazol-5-yl)phenol

To a solution of 5-(4-(benzyloxy)-3-fluorophenyl)-1-methyl-1H-tetrazole (500 mg, 1.76 mmol) in 6 mL of ethanol and 6 mL of tetrahydrofuran was added formic acid (0.7 mL, 17.6 mmol) followed by palladium black (281 mg, 2.64 mmol): The reaction mixture was stirred at room temperature for 4 hours. The reaction mixture was filtered through Celite® and the filtrate was concentrated in vacuo to give 2-fluoro-4-(1-methyl-1H-tetrazol-5-yl)phenol as a white solid (330 mg) which was used for in subsequent reactions without further purification.

### Preparation 22: 4-(1-Methyl-1H-tetrazol-5-yl)phenol

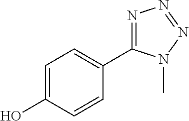

### A) 4-(Benzyloxy)-N-methylbenzamide

To a flask charged with thionyl chloride (0.35 mL, 4.82 mmol) was added a solution of commercially available 4-benzyloxybenzoic acid (1.00 g, 4.38 mmol) in 10 mL of dichloromethane and 0.01 mL of N,N-dimethylformamide at zero degrees Celsius with stirring. The ice bath was removed and the solution was stirred for 4 hours at room temperature. The mixture was concentrated in vacuo to give a white solid. This solid was taken up in 10 mL of methyl amine (2 M in tetrahydrofuran) and the resulting solution was stirred at room temperature for 70 hours. The mixture was diluted with ethyl acetate and water and the organic layer was separated, dried over magnesium sulfate, filtered and the filtrate was concentrated in vacuo to give a white solid. This solid was recrystallized from ethyl acetate and heptane to give 4-(benzyloxy)-N-methylbenzamide as white needles (850 mg).

### B) 5-(4-(Benzyloxy)phenyl)-1-methyl-1H-tetrazole

To a stirred solution of 4-(benzyloxy)-N-methylbenzamide (200 mg, 0.829 mmol) in 3 mL of acetonitrile and one drop of N,N-dimethylformamide, in a flask topped with a reflux condenser, was added triethylamine (0.12 mL) under a nitrogen atmosphere. The reaction mixture was stirred for 10 minutes before thionyl chloride (0.078 mL, 1.08 mmol) was added drop-wise. The yellow reaction mixture was stirred for 1 hour at room temperature under a nitrogen atmosphere. Triethylamine (0.36 mL) was then added slowly, followed by tetrabutylammonium chloride (37.4 mg, 0.12 mmol) and sodium azide (611 mg, 1.82 mmol). The resulting yellow suspension was vigorously stirred for 70 hours at room temperature under a nitrogen atmosphere. The mixture was diluted with water and ethyl acetate. The organic layer was separated, washed with brine, dried over magnesium sulfate, filtered and the filtrate was concentrated in vacuo. The residue was purified by flash chromatography, eluting with a gradient of 10 to 40% ethyl acetate in heptane to give 5-(4-(benzyloxy)phenyl)-1-methyl-1H-tetrazole as a white solid (180 mg).

### C) 4-(1-Methyl-1H-tetrazol-5-yl)phenol

To a stirred solution 5-(4-(benzyloxy)phenyl)-1-methyl-1H-tetrazole (180 mg, 0.676 mmol) in 3 mL of ethanol and 3 mL of tetrahydrofuran was added formic acid (0.27 mL, 6.76 mmol) followed by palladium black (108 mg, 1.01 mmol). The mixture was stirred at room temperature for 4 hours. The reaction mixture was filtered through Celite® and the filtrate was concentrated to give 4-(1-methyl-1H-tetrazol-5-yl)phenol as a white solid (110 mg), which was used in subsequent reactions without further purification.

### Preparation 23: 3-Fluoro-4-hydroxybenzamide

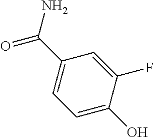

A mixture of commercially available 3-fluoro-4-hydroxybenzonitrile (500 mg, 3.65 mmol) and potassium hydroxide (1.02 g, 18.2 mmol) in 10 mL of 80% ethanol was heated at reflux for 16 hours. After cooling to room temperature the mixture was concentrated in vacuo and the residue was taken up into water, acidified with acetic acid and extracted with ethyl acetate. The combined organic extracts were dried over magnesium sulfate, filtered and the filtrate was concentrated in vacuo. The residue was purified by flash chromatography, eluting with a gradient of 20 to 60% ethyl acetate in heptane to give 3-fluoro-4-hydroxybenzamide as a white solid (210 mg).

Alternatively, 3-fluoro-4-hydroxybenzamide can be prepared as follows:

To a stirred solution of urea hydrogen peroxide (4.2 g, 43.8 mmol) in 12 mL of water was added solid sodium hydroxide (1.04 g, 25.5 mmol). The resulting solution was cooled in an ice bath before a solution of 3-fluoro-4-hydroxybenzonitrile (1.00 g, 7.29 mmol)in 5 mL of ethanol was added. The mixture was vigorously stirred for 2 hours at room temperature before it was diluted with water (100 mL) and ethyl acetate (100 mL). The mixture was stirred for 5 minutes before 1 M hydrochloric acid was added until pH 4. The aqueous layer was separated and extracted with ethyl acetate (3×100 mL). The combined organic layers were dried over magnesium sulfate, filtered, and the filtrate was concentrated to give a white solid. This solid was triturated with diethyl ether and heptane (2:1, 90 mL) for 1 hour, before filtering to give 3-fluoro-4-hydroxybenzamide as a white solid (1.05 g). ¹H NMR (400 MHz, deutero dimethyl sulfoxide) delta 6.93 (t, J=8.69 Hz, 1H) 7.19 (br. s., 1H) 7.53 (dd, J=8.39, 1.95 Hz, 1H) 7.62 (dd, J=12.40, 2.05 Hz, 1H) 7.77 (br. s., 1H) 10.39 (s, 1H). LCMS (ES) 156.0 (M+1).

### Preparation 24: 2-Fluoro-4-hydroxybenzamide

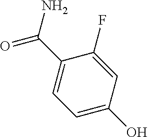

To a stirred solution of urea hydrogen peroxide (2.1 g, 21.9 mmol) in 6 mL of water was added solid sodium hydroxide (521 mg, 12.8 mmol). The resulting solution was cooled in an ice bath before a solution of 2-fluoro-4-hydroxybenzonitrile (500 mg, 3.65 mmol) in 2 mL of ethanol was added. The mixture was vigorously stirred for 2 hours at room temperature before it was diluted with water (100 mL) and ethyl acetate (100 mL). The mixture was stirred for 5 minutes before 1 M hydrochloric acid was added until pH=4. The aqueous layer was separated and extracted with ethyl acetate (3×50 mL). The combined organic layers were dried over magnesium sulfate, filtered, and the filtrate was concentrated to give 2-fluoro-4-hydroxybenzamide as a white solid.

### Preparation 25: Isopropyl 4-(5-cyano-4-(1-hydroxyethyl)-1H-pyrazol-1-yl)piperidine-1-carboxylate

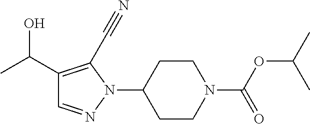

Isopropyl 4-(5-cyano-4-formyl-1H-pyrazol-1-yl)piperidine-1-carboxylate (Example 9, Step A) (51 mg, 0.18 mmol) was dissolved in 2 mL of anhydrous tetrahydrofuran and cooled to negative 78 degrees Celsius under a nitrogen atmosphere. Methylmagnesium bromide (0.070 mL, 0.21 mmol, 3 M in diethyl ether) was then added drop-wise. The cold bath was removed and the mixture was stirred for 1 hour at room temperature. The mixture was diluted with 1 M aqueous potassium bisulfate and extracted three times with ethyl acetate. The combined organic extracts were washed with brine, dried over sodium sulfate, filtered and the filtrate was concentrated in vacuo. The residue was purified by flash chromatography, eluting with a gradient of ethyl acetate in heptane (20 to 100% ethyl acetate) to give isopropyl 4-(5-cyano-4-(1-hydroxyethyl)-1H-pyrazol-1-yl)piperidine-1-carboxylate as a white solid (33 mg) which was used in subsequent steps without purification.

### Preparation 26: 1-Methylcyclopropyl 4-nitrophenyl carbonate

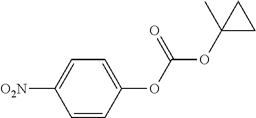

### A) 1-Methylcyclopropanol

A 1 L flask was charged with titanium methoxide (100 g), cyclohexanol (232 g), and toluene (461 mL). The flask was equipped with a Dean-Stark trap and condenser. The mixture was heated at 140 degrees Celsius until the methanol was removed. The toluene was removed at 180 degrees Celsius. More toluene was added and this process was repeated twice. After all the toluene was removed the flask was dried under high vacuum. Diethyl ether (580 mL) was added to the flask to prepare a 1 M solution in diethyl ether. A 5 L, 3-neck flask was equipped with an overhead stirrer, inert gas inlet and a pressure-equalizing addition funnel. The flask was flushed with nitrogen gas and charged with methyl acetate (60.1 mL, 756 mmol), titanium cyclohexyloxide (1 M solution in ether 75.6 mL), and diethyl ether (1500 mL). The solution was stirred while keeping the reaction flask in a room temperature water bath. The addition funnel was charged with the 3 M ethylmagnesium bromide solution (554 mL, 1.66 moles). The Grignard reagent was added drop-wise over 3 hours at room temperature. The mixture became a light yellow solution, and then gradually a precipitate formed which eventually turned to a dark green/brown/black colored mixture. After stirring for an additional 15 minutes, following the addition of the Grignard, the mixture was carefully poured into a mixture of 10% concentrated sulfuric acid in 1 L of water. The resulting mixture was stirred until all the solids dissolved. The aqueous layer was separated and extracted with diethyl ether 2×500 mL. The combined organic extracts were washed sequentially with water, brine, dried over potassium carbonate (500 g) for 30 minutes, filtered and the filtrate was concentrated in vacuo to an oil. Sodium bicarbonate (200 mg) was added and the crude material was distilled, collecting fractions boiling around 100 degrees Celsius to give the title compound (23 grams) with methyl ethyl ketone and 2-butanol as minor impurities. ¹H NMR (500 MHz, deuterochloroform) delta 0.45 (app. t, J=6.59 Hz, 2H), 0.77 (app. t, J=5.61 Hz, 2H), 1.46 (s, 3H). The preparation of the title compound is also described in WO09105717.

### B) 1-Methylcyclopropyl 4-nitrophenyl carbonate

A solution of 1-methylcyclopropanol (10 g, 137 mmol), 4-nitrophehyl chloroformate (32 g, 152 mmol), and a few crystals of 4-dimethylaminopyridine (150 mg, 1.2 mmol) in dichloromethane (462 mL), was cooled to zero degree Celsius. Triethylamine (36.5 g, 361 mmol) was added drop-wise. After 10 minutes, the ice bath was removed and the reaction was allowed to stir at room temperature for 14 hours. The reaction mixture was washed twice with saturated aqueous sodium carbonate. The aqueous phase was extracted with dichloromethane. The combined organic extracts were washed with water, dried over magnesium sulfate, filtered and the filtrate concentrated in vacuo. The residue was purified by flash silica gel chromatography, eluting with a gradient mixture of ethyl acetate in heptane (0 to 5% ethyl acetate over the first 10 minutes, then isocratic at 5% ethyl acetate to heptane) to give 20.8 g of the desired carbonate as a clear oil. This oil solidified upon standing.

¹H NMR (500 MHz, deuterochloroform) delta 0.77 (app. t, J=6.59 Hz, 2H), 1.09 (app. t, J=7.07 Hz, 2H), 1.67 (s, 3H), 7.40 (app. dt, J=9.27, 3.17 Hz, 2H), 8.29 (app. dt, J=9.27, 3.17 Hz, 2H).

Alternatively the 1-methylcyclopropanol can be prepared as follows:

### 1-Methylcyclopropanol

A 2000 mL 4-neck flask was equipped with a mechanical stirrer, inert gas inlet, thermometer, and two pressure-equalizing addition funnels. The flask was flushed with nitrogen and charged with 490 mL of diethyl ether followed by 18.2 mL (30 mmol) of titanium tetra(2-ethylhexyloxide). One addition funnel was charged with a solution prepared from 28.6 mL (360 mmol) of methyl acetate diluted to 120 mL with ether. The second addition funnel was charged with 200 mL of 3 M ethylmagnesium bromide in ether solution. The reaction flask was cooled in an ice water bath to keep the internal temperature at 10 degrees Celsius or below. Forty milliliters of the methyl acetate solution was added to the flask. The Grignard reagent was then added drop-wise from the addition funnel at a rate of about 2 drops every second, and no faster than 2 mL per minute. After the first 40 mL of Grignard reagent had been added, another 20 mL portion of methyl acetate in ether solution was added. After the second 40 mL of Grignard reagent had been added, another 20 mL portion of methyl acetate in diethyl ether solution was added. After the third 40 mL of Grignard reagent had been added, another 20 mL portion of methyl acetate in ether solution was added. After the fourth 40 mL of Grignard reagent had been added, the last 20 mL portion of methyl acetate in ether solution was added.

The mixture was stirred for an additional 15 minutes following the completion of the addition of Grignard reagent. The mixture was then poured into a mixture of 660 g of ice and 60 mL of concentrated sulfuric acid with rapid stirring to dissolve all solids. The phases were separated and the aqueous phase was extracted again with 50 mL of diethyl ether. The combined ether extracts were washed with 15 mL of 10% aqueous sodium carbonate, 15 mL of brine, and dried over 30 grams magnesium sulfate for 1 hour with stirring. The ether solution was then filtered. Tri-n-butylamine (14.3 mL, 60 mmol) and mesitylene (10 mL were added. Most of the diethyl ether was removed by distillation at atmospheric pressure using a 2.5 cm×30 cm jacketed Vigreux column. The remaining liquid was transferred to a smaller distillation flask using two 10 mL portions of hexane to facilitate the transfer. Distillation at atmospheric pressure was continued through a 2 cm×20 cm jacketed Vigreux column. The liquid distilling at 98-105° C. was collected to provide 14 g of the title compound as a colorless liquid. ¹H NMR (400 MHz, deuterochloroform) delta 0.42-0.48 (m, 2H), 0.74-0.80 (m, 2H), 1.45 (s, 3H), 1.86 (br. s., 1H).

### Preparation 27: 2-fluoro-4(1-methyl-1H-imidazol-5-yl)phenol

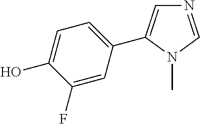

### A) 5-(3-Fluoro-4-methoxyphenyl)-1-methyl-1H Imidazole

2-Fluoro-4-bromo anisole (0.216 mL, 1.63 mmol), tri(2-furyl)phosphine (25.9 mg, 0.108 mmol), and potassium carbonate (300 mg, 2.17 mmol) were placed in a microwave vial and dissolved in anhydrous N,N-dimethylformamide (4.8 mL). The mixture was degassed with a stream of nitrogen gas for 10 minutes, 1-methylimidazole (0.087 mL, 1.1 mmol) and palladium(II) acetate (12.4 mg, 0.054 mmol) were added, and the mixture was degassed for another 10 minutes. The vessel was placed in a microwave reactor at 140 degrees Celsius for 2 hours. The mixture was diluted with ethyl acetate, filtered through Celite®, and the filtrate was concentrated under reduced pressure. The crude material was purified by chromatography eluting with a 25 to 100% ethyl acetate in heptane then 0 to 10% methanol in dichloromethane gradient to give the title compound as a yellow oil (210 mg). ¹H NMR (500 MHz, deuterochloroform) delta 3.57 (s, 3H), 3.85 (s, 3H), 6.95-6.98 (m, 2H), 7.00-7.07 (m, 2H), 7.42 (s, 1H). Proton shift at 7.42 is indicative of desired imidazole isomer as compared to literature (Eur. J. Org. chem., 2008, 5436 and Eur. J. Org., 2006, 1379).

### B) 2-Fluoro-4-(1-methyl-1H-imidazol-5-yl)phenol

To a solution of 5-(3-fluoro-4-methoxyphenyl)-1-methyl-1H Imidazole (101.8 mg, 0.494 mmol) in dichloromethane (2.0 mL) was added a solution of boron(III) bromide (0.50 mL, 1.0 M solution in heptane) at −30 degrees Celsius. The mixture was stirred at room temperature for 20 hours. The mixture was then cooled to −30 degrees Celsius and methanol (2 mL) was added to the mixture. The mixture was concentrated in vacuo, and the residue was dissolved in water and neutralized with 1M sodium hydroxide. The solution was concentrated to give the title compound as a yellow solid (90 mg). This compound was used further without purification.

### Preparation 28: 2-Fluoro-4-(1-methyl-1H-imidazol-2-yl)phenol

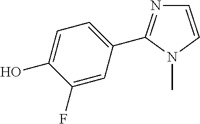

### A) 2-(3-Fluoro-4-methoxyphenyl)-1-methyl-1H Imidazole

2-Fluoro-4-bromoanisole (0.256 mL, 1.93 mmol) and copper(I) iodide (375 mg, 1.93 mmol) were placed in a microwave vial and dissolved in N,N-dimethylformamide (4.8 mL). The mixture was degassed for 10 minutes with a stream of nitrogen gas, 1-methylimidazole (0.078 mL, 0.96 mmol) and palladium(II) acetate (11 mg, 0.048 mmol) were added, and the mixture was degassed for another 10 minutes. The vessel was placed in a microwave reactor at 140 degrees Celsius for 2 hours. The mixture was diluted with ethyl acetate (3 mL), poured into saturated aqueous ammonium chloride solution, stirred in the open air for 30 minutes, and extracted twice with ethyl acetate. The combined organic phases were washed with water, dried over sodium sulfate, filtered and the filtrate was concentrated in vacuo. The crude material was purified by chromatography, eluting with a gradient mixture of ethyl acetate to heptane (25 to 100% ethyl acetate/heptane then 0 to 10% methanol in dichloromethane) to give 2-(3-fluoro-4-methoxyphenyl)-1-methyl-1H Imidazole as a yellow oil (35.8 mg). ¹H NMR (400 MHz, deuterochloroform) delta 3.66 (s, 3H), 3.88 (s, 3H), 6.90 (s, 1H), 6.96 (m 1H), 7.10 (s, 1H), 7.24-7.33 (m, 2H). Proton NMR indicates desired imidazole isomer as compared to the proton NMR of 5-(3-fluoro-4-methoxyphenyl)-1-methyl-1H Imidazole (preparation 27) and the literature Eur. J. Org. chem., 2008, 5436 and Eur. J. Org., 2006, 1379).

### B) 2-Fluoro-4-(1-methyl-1H-imidazol-2-yl)phenol

2-Fluoro-4-(1-methyl-1H-imidazol-2-yl)phenol was prepared from 2-(3-fluoro-4-methoxyphenyl)-1-methyl-1H Imidazole following a procedure analogous to that in Preparation 27 (B) to give the title compound as a brown solid (33.4 mg). The crude material was used further without purification.

### Preparation 29: 2-Fluoro-4-(methylsulfonyl)-1-(prop-1-en-2-yl)benzene

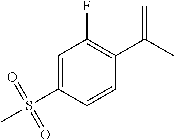

To a solution of 1-bromo-2-fluoro-4-(methylsulfonyl)benzene (199 mg, 0.790 mmol) and potassium isopropenyltrifluoroborate (300 mg, 2.57 mmol) in 2-propanol (10 mL) was added the catalyst 1,1′-bis-(diphenylphosphino)-ferrocene palladium dichloride (67 mg, 0.089 mmol) and triethylamine (0.17 mL, 1.20 mmol) sequentially. The reaction was heated at 90 degrees Celsius for 15 hours, and then the reaction was stirred at room temperature for 48 hours. Water and ethyl acetate were then added, and the layers were separated. The aqueous layer was extracted with ethyl acetate. The organic extracts were combined, washed with brine and dried over sodium sulfate. The mixture was filtered, and the filtrate concentrated under reduced pressure. The residue was purified by silica gel chromatography (10 to 100% ethyl acetate in heptane) to give the title compound as a white solid (130 mg, 80%). ¹H NMR (500 MHz, deuterochloroform) delta 2.17 (s, 3H), 3.08 (s, 3H), 5.29-5.43 (m, 2H), 7.51 (t, J=7.56 Hz, 1H), 7.64 (dd, J=9.88, 1.59 Hz, 1H), 7.70 (dd, J=8.05, 1.71 Hz, 1H).

### Example 1

### Isopropyl 4-[5-cyano-4-({2-fluoro-4-[(2-hydroxyethyl)sulfonyl]-phenoxy}methyl)-1H-pyrazol-1-yl]piperidine-1-carboxylate

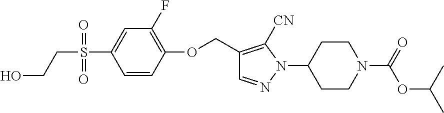

### A) Isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate

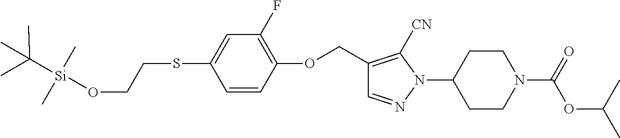

To a solution of isopropyl 4-[5-cyano-4-(hydroxymethyl)-1H-pyrazol-1-yl]piperidine-1-carboxylate (54.4 mg, 0.19 mmol), 4-[(2-{[tert-butyl(dimethyl)silyl]oxy}ethyl)thio]-2-fluorophenol (64 mg, 0.21 mmol) and polymer-bound triphenylphosphine (3 mmol/g, 310 mg, 0.93 mmol) in 1,4-dioxane (1.7 mL) was added dropwise diethyl azodicarboxylate (0.033 mL, 0.205 mmol). The reaction mixture was stirred 16 hours under an atmosphere of nitrogen. The polymer was filtered off, and the filtrate was then evaporated under vacuum. The residue was purified by chromatography over silica gel eluting with 20% to 70% ethyl acetate in heptane to give the title compound as an oil (44 mg, 41%). ¹H NMR (400 MHz, deuterochloroform) delta 0.02 (s, 6H), 0.86 (s, 9H), 1.24 (d, J=6.3 Hz, 6H), 2.00 (br d, 2H), 2.06-2.17 (m, 2H), 2.86-3.01 (m, 4H), 3.72-3.77 (m, 2H), 4.10-4.22 (m, 2H), 4.45-4.53 (m, 1H), 4.90 (m, 1H), 5.06 (s, 2H) 6.90-6.96 (m, 1H), 7.05-7.10 (m, 1H), 7.12-7.16 (m, 1H), 7.65 (s, 1H).

### B) Isopropyl 4-[5-cyano-4-({2-fluoro-4-[(2-hydroxyethyl)sulfonyl]phenoxy}methyl)-1H-pyrazol-1-yl]piperidine-1-carboxylate

To a solution of isopropyl 4-[4-({4-[(2-{[tert-butyl(dimethyl)silyl]oxy}ethyl)thio]-2-fluorophenoxy}methyl)-5-cyano-1H-pyrazol-1-yl]piperidine-1-carboxylate (44 mg, 0.076 mmol) in dichloromethane (2 mL) was added a 4 M solution of hydrogen chloride in 1,4-dioxane (0.2 mL, 0.76 mmol). The resulting mixture was stirred for 16 hours at room temperature. The solvent was evaporated, and the residue was dried under vacuum. The residue was taken up in dichloromethane (1 mL) and 3-chloroperbenzoic acid (48 mg, 0.21 mmol) was added. The resulting solution was stirred at room temperature for 1 hour. The reaction mixture was diluted with dichloromethane, and the organic phase was washed with a saturated aqueous sodium carbonate solution and then brine. The organic phase was dried over magnesium sulfate and filtered. The filtrate was evaporated under reduced pressure, and the residue purified by chromatography on silica gel (70% to 100% ethyl acetate in heptane) to give the titled compound as an oil (15 mg, 40%). ¹H NMR (400 MHz, deuterochloroform) delta 1.24 (d, J=6.3 Hz, 6H), 2.02 (br d, 2H), 2.07-2.19 (m, 2H), 2.94 (br t, 2H), 3.30-3.36 (m, 2H), 3.97-4.03 (m, 2H), 4.31 (br s, 2H), 4.47-4.56 (m, 1H), 4.93 (m, 1H), 5.18 (s, 2H), 7.14-7.22 (m, 1H), 7.63-7.74 (m, 3H); LCMS (ES+): 495.0 (M+H).

### Example 2

### Isopropyl 4-(5-cyano-4-{[2-fluoro-4-(methylsulfonyl)phenoxy]methyl}-1H-pyrazol-1-yl)piperidine-1-carboxylate

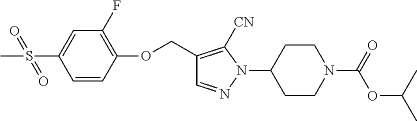

### A) Isopropyl 4-(5-bromo-4-{[2-fluoro-4-(methylsulfonyl)phenoxy]methyl}-1H-pyrazol-1-yl)piperidine-1-carboxylate

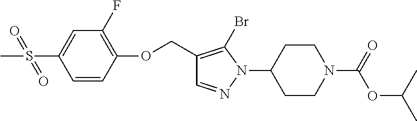

This compound was prepared from 2-fluoro-4-(methylsulfonyl)phenol (WO 2007054668) and isopropyl 4-[5-bromo-4-(hydroxymethyl)-1H-pyrazol-1-yl]piperidine-1-carboxylate (Preparation 4) in a manner similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The compound was purified by chromatography on silica gel (60% ethyl acetate in hexane). ¹H NMR (400 MHz, deuterochloroform) delta 7.70 (ddd, 1H), 7.67 (s, 1H), 7.65 (dd; 1H), 7.18 (t, 1H), 5.03 (s, 2H), 4.92 (m, 1H), 4.43 (m, 1H), 4.31 (br s, 2H), 3.03 (s, 3H), 2.91 (br t, 2H), 2.09 (m, 2H), 1.92 (br d, 2H), 1.25 (d, 6H).

### B) Isopropyl 4-(5-cyano-4-{[2-fluoro-4-(methylsulfonyl)phenoxy]methyl}-1H-pyrazol-1-yl)piperidine-1-carboxylate

A mixture of isopropyl 4-(5-bromo-4-{[2-fluoro-4-(methylsulfonyl)phenoxy]methyl}-1H-pyrazol-1-yl)piperidine-1-carboxylate (237 mg, 0.46 mmol) and copper cyanide (82 mg, 0.91 mmol) in anhydrous N,N-dimethylformamide (4.0 mL) was heated at 165° C. for 24 hours under an argon atmosphere. The resulting dark brown mixture was allowed to cool to room temperature and was then poured carefully into a stirred solution of ferric chloride hexahydrate (618 mg, 2.29 mmol), concentrated aqueous hydrochloric acid (2 mL) and water (10 mL). Ethyl acetate (10 mL) was added, and the resulting mixture was stirred at 65° C. for 30 minutes. The mixture was allowed to cool to room temperature and was then extracted with ethyl acetate three times. The combined extracts were washed sequentially with 2 M aqueous hydrochloric acid solution, 2 M aqueous sodium hydroxide solution, water and brine and then dried over magnesium sulfate. The mixture was filtered, and the filtrate was concentrated under vacuum to give the crude product as a brown oil that was purified by chromatography on silica gel (60% ethyl acetate in hexane) to give isopropyl 4-(5-cyano-4-{[2-fluoro-4-(methylsulfonyl)phenoxy]methyl}-1H-pyrazol-1-yl)piperidine-1-carboxylate as a pale yellow oil which solidified on standing (77 mg, 36%). ¹H NMR (400 MHz, deuterochloroform) delta 7.72-7.70 (m, 1H), 7.69 (s, 1H), 7.67 (dd, 1H), 7.18 (t, 1H), 5.18 (s, 2H), 4.92 (m, 1H), 4.52 (m, 1H), 4.31 (br s, 2H), 3.04 (s, 3H), 2.93 (br t, 2H), 2.12 (m, 2H), 2.01 (br d, 2H), 1.25 (d, 6H); LCMS (ES+): 465.06 (M+1).

### Example 3

### Isopropyl 4-(5-cyano-4-{[2-fluoro-4-(1H-tetrazol-1-yl)phenoxy]-methyl}-1H-pyrazol-1-yl)piperidine-1-carboxylate

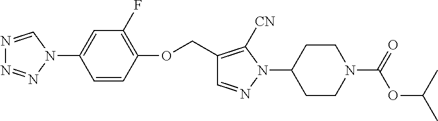

This compound was prepared from 2-fluoro-4-(1H-tetrazol-1-yl)phenol (Preparation 9) and isopropyl 4-[5-cyano-4-(hydroxymethyl)-1H-pyrazol-1-yl]piperidine-1-carboxylate (Preparation 5) in a manner similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The crude product was purified by chromatography on silica gel (50% to 70% ethyl acetate in heptane). ¹H NMR (400 MHz, deuterochloroform) delta 1.24 (d, J=6.3 Hz, 6H), 2.01 (br d, 2H), 2.07-2.18 (m, 2H), 2.93 (br t, 2H), 4.32 (br s, 2H), 4.46-4.56 (m, 1H), 4.91 (septet, 1H), 5.18 (s, 2H), 7.16-7.26 (m, 1H), 7.42-7.49 (m, 1H), 7.49-7.58 (m, 1H), 7.69 (s, 1H), 8.93 (s, 1H); LCMS (ES+): 455.1 (M+H).

### Example 4

### Isopropyl 4-(5-cyano-4-{[4-(1H-tetrazol-1-yl)phenoxy]methyl}-1H-pyrazol-1-yl)piperidine-1-carboxylate

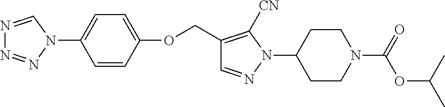

This compound was prepared from 4-(1H-tetrazol-1-yl)phenol and isopropyl 4-[5-cyano-4-(hydroxymethyl)-1H-pyrazol-1-yl]piperidine-1-carboxylate (Preparation 5) in a manner similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The crude product was purified by chromatography over silica gel using a gradient of 50% to 70% ethyl acetate in heptane. ¹H NMR (400 MHz, deuterochloroform) delta 1.24 (d, J=6.3 Hz, 6H), 2.02 (br d, 2H), 2.07-2.20 (m, 2H), 2.94 (br t, 2H), 4.32 (br s, 2H), 4.47-4.56 (m, 1H), 4.88-4.97 (m, 1H), 5.11 (s, 2H), 7.11-7.16 (m, 2H); 7.59-7.65 (m, 2H), 7.68 (s, 1H), 8.90 (s, 1H); LCMS (ES+): 437.0 (M+H).

### Example 5

### Isopropyl 4-(5-cyano-4-{[(2-methylpyridin-3-yl)oxy]methyl}-1H-pyrazol-1-yl)piperidine-1-carboxylate

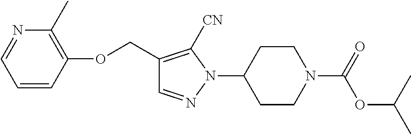

This compound was prepared from 2-methylpyridin-3-ol and isopropyl 4-[5-cyano-4-(hydroxymethyl)-1H-pyrazol-1-yl]piperidine-1-carboxylate (Preparation 5) in a manner similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The crude material was purified by preparative reverse phase HPLC on a Phenomenex Gemini C₁₈ 21.2×150 mm, 0.005 mm column eluting with a gradient of water in methanol (0.1% ammonium hydroxide as modifier). ¹H NMR (400 MHz, deuterochloroform) delta 1.25 (d, J=6.4 Hz, 6H), 2.02 (br d, 2H), 2.06-2.21 (m, 2H), 2.52 (s, 3H), 2.94 (br t, 2H), 4.33 (br s, 2H), 4.47-4.57 (m, 1H), 4.91 (m, 1H), 5.06 (s, 2H), 7.11-7.22 (m, 2H), 7.66 (s, 1H), 8.11-8.19 (m, 1H); LCMS (ES+): 384.1 (M+H).

### Example 6

### Isopropyl 4-(5-cyano-4-{[(2-methylpyridin-3-yl)amino]methyl}-1H-pyrazol-1-yl)piperidine-1-carboxylate

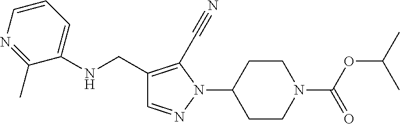

To a flask charged with isopropyl 4-(5-cyano-4-formyl-1H-pyrazol-1-yl)piperidine-1-carboxylate (Example 9, Step A) (50 mg, 0.17 mmol) and 2-methylpyridin-3-amine (19 mg, 0.17 mmol) was added 2 mL of dichloroethane followed by N,N-diisopropylethylamine (0.03 mL, 0.17 mmol). The flask was flushed with nitrogen gas and titanium isopropoxide (97.8 mg, 0.34 mmol) was added at room temperature. The reaction mixture was stirred at this temperature for 19 hours before sodium triacetoxyborohydride (75.2 mg, 0.34 mmol) was added. The mixture was stirred for 24 hours at room temperature. The reaction mixture was diluted with dichloromethane and saturated aqueous bicarbonate was added. The mixture was filtered through a pad of Celite®. The filtrate layers were separated and the aqueous phase was extracted once with dichloromethane. The combined organic layers were dried over magnesium sulfate, filtered and the filtrate was concentrated in vacuo. The residue was purified by flash chromatography, eluting with a gradient mixture of ethyl acetate in heptane (60 to 80% ethyl acetate). Proton NMR showed that the material was the imine. The imine was then dissolved in 2 mL of methanol and 1 mL of tetrahydrofuran, and the mixture was cooled to zero degrees Celsius. Sodium borohydride (10 mg, 0.26 mmol) was added and the ice bath was removed. The mixture was stirred for at room temperature for 4 hours before saturated aqueous sodium bicarbonate was added. The mixture was partially concentrated in vacuo and the aqueous mixture was extracted once with ethyl acetate. The organic extracts were concentrated in vacuo and the residue was purified by flash chromatography, eluting with a gradient mixture of ethyl acetate in heptane (80 to 100% ethyl acetate) to give the title compound (24 mg, 63% yield). ¹H NMR (400 MHz, deuterochloroform) delta 1.15-1.33 (m, 6H), 1.93-2.04 (m, 2H), 2.11 (qd, J=12.2, 4.6 Hz, 2H), 2.43 (s, 3H), 2.84-2.99 (m, 2H), 3.94 (t, J=5.2 Hz, 1H), 4.31 (br. s., 2H), 4.37 (d, J=5.5 Hz, 2H), 4.41-4.54 (m, 1H), 4.86-4.98 (m, 1H), 6.78-6.87 (m, 1H), 6.96-7.07 (m, 1H), 7.52-7.62 (m, 1H), 7.87-7.97 (m, 1H). LCMS (ES+) 383.1 (M+1).

### Example 7

### Isopropyl 4-(5-cyano-4-{[4-(dimethoxyphosphoryl)-2-fluorophenoxy]methyl}-1H-pyrazol-1-yl)piperidine-1-carboxylate

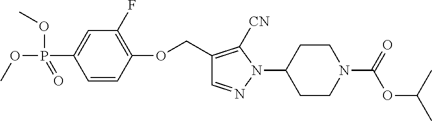

### A) Isopropyl 4-{4-[(4-bromo-2-fluorophenoxy)methyl]-5-cyano-1H-pyrazol-1-yl}piperidine-1-carboxylate

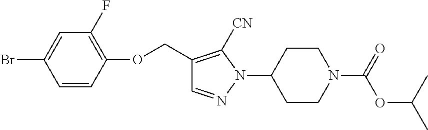

This compound was prepared from 4-bromo-2-fluorophenol and isopropyl 4-[5-cyano-4-(hydroxymethyl)-1H-pyrazol-1-yl]piperidine-1-carboxylate (Preparation 5) in a manner similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The crude compound was purified by chromatography on silica gel eluting with 0% to 100% ethyl acetate in heptane. ¹H NMR (deuterochlorform): delta 1.35 (6H, d), 2.1 (2H, m), 2.2 (2H, m) 3.0 (2H, m), 4.3 (2H, m), 4.5 (1H, m), 4.95 (1H, m), 5.15 (2H, s), 6.95 (1H, d,d), 7.2 (1H,d), 7.3 (1H, d), 7.7 (1H, s); LCMS (ES+): 464.8 (M−1).

### B) Isopropyl 4-(5-cyano-4-{[4-(dimethoxyphosphoryl)-2-fluorophenoxy]methyl}-1H-pyrazol-1-yl)piperidine-1-carboxylate

Isopropyl 4-{4-[(4-bromo-2-fluoro-phenoxy)methyl]-5-cyano-1H-pyrazol-1-yl}piperidine-1-carboxylate (200 mg, 0.711 mmol) was dissolved in degassed tetrahydrofuran (5 mL). Tetrakis triphenylphosphine palladium (0) (170 mg, 0.144 mmol) and dimethyl phosphite (0.084 mL, 0.875 mmol) were added followed by triethylamine (0.152 mL, 1.09 mmol). The vessel was capped, and the reaction mixture was heated at 75° C. for 5 hours. The solvent was evaporated under vacuum, and the crude product was purified by preparative reverse phase HPLC on a Waters XBridge C₁₈ 19×100 mm, 5 μm column eluting with 80% water/20% acetonitrile to 100% acetonitrile (0.03% ammonium hydroxide modifier). Analytical LCMS: retention time 1.06 minutes (Acquity HSS T3 2.1×50 mm, 1.8 μm column; 95% water/acetonitrile linear gradient to 5% water/acetonitrile over 1.8 minutes, held at 5% water/acetonitrile for 2.0 minutes; 0.05% trifluoroacetic acid modifier; flow rate 1.3 mL/minute); LCMS (ES+): 495.1 (M+H).

### Example 8

### Isopropyl 4-{5-cyano-4-[(4-cyano-2-fluorophenoxy)methyl]-1H-pyrazol-1-yl}piperidine-1-carboxylate

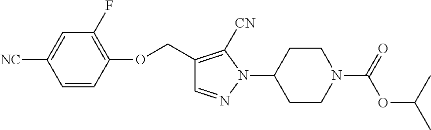

This compound was prepared from 4-cyano-3-fluorophenol and isopropyl 4-[5-cyano-4-(hydroxymethyl)-1H-pyrazol-1-yl]piperidine-1-carboxylate (Preparation 5) in a manner 20. similar to that described for the preparation of isopropyl 4-[4-({4-[(2-{[tert-butyl(dimethyl)silyl]oxy}ethyl)thio]-2-fluorophenoxy}-methyl)-5-cyano-1H-pyrazol-1-yl]piperidine-1-carboxylate (Example 1, Step A, Mitsunobu reaction). The crude product was purified by preparative HPLC on a Waters XBridge C₁₈ column 19×100 mm, 5 μm column eluting with a gradient of water in acetonitrile (0.03% ammonium hydroxide modifier). Analytical LCMS: retention time 3.39 minutes (Atlantis C₁₈ 4.6×50 mm, 5 μm column; 80% H₂O/acetonitrile linear gradient to 5% water/acetonitrile for 4.0 minutes; 0.05% trifluoroacetic acid modifier; flow rate 2.0 mL/minute); LCMS (ES+): 412 (M+H).

### Example 9

### Isopropyl 4-(5-cyano-4-{2-[2-fluoro-4-(methylsulfonyl)phenyl]ethyl}-1H-pyrazol-1-yl)piperidine-1-carboxylate

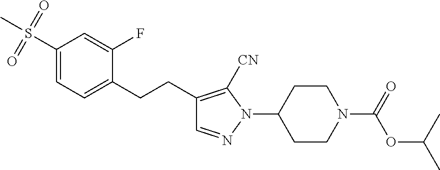

### Step A: Isopropyl 4-(5-cyano-4-formyl-1H-pyrazol-1-yl)piperidine-1-carboxylate

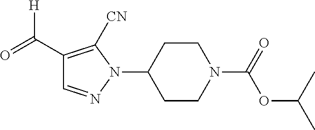

Into a solution of isopropyl 4-[5-cyano-4-(hydroxymethyl)-1H-pyrazol-1-yl]piperidine-1-carboxylate (Preparation 5) (400 mg, 1.37 mmol) in anhydrous dichloromethane (10 mL) at 0° C. was added trichloroisocyanuric acid (379 mg, 1.63 mmol) and 2,2,6,6-tetramethylpiperidine-1-oxyl (TEMPO, 22 mg, 0.14 mmol). The yellow mixture was removed from the ice bath and stirred at room temperature for 45 minutes. The reaction mixture was filtered through a Celite™ pad, which was washed with dichloromethane. The filtrate was combined with saturate aqueous sodium bicarbonate, and the layers separated. The aqueous layer was extracted with dichloromethane. Both of the organic solutions were combined and washed with brine and dried over sodium sulfate. The mixture was filtered, and the filtrate was concentrated under reduced pressure to give an oily mixture which was purified by chromatography on silica gel (5-100% ethyl acetate in heptane) to give isopropyl 4-(5-cyano-4-formyl-1H-pyrazol-1-yl)piperidine-1-carboxylate as a clear oil that partially solidified under vacuum (311 mg, 78%). ¹H NMR (400 MHz, deuterochloroform) delta 10.0 (s, 1H), 8.06 (s, 1H), 4.99-4.94 (m, 1H), 4.65-4.59 (m, 1H), 4.37 (m, 2H), 2.98-2.95 (m, 2H), 2.23-2.15 (m, 2H), 2.06-2.04 (m, 2H), 1.28 (d, 6H, J=6.3 Hz); LCMS (ES+): 290.1 (M+).

### Step B: Isopropyl 4-(5-cyano-4-ethynyl-1H-pyrazol-1-yl)piperidine-1-carboxylate

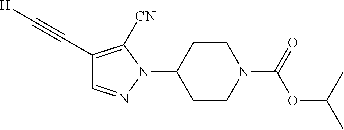

Into a solution of isopropyl 4-(5-cyano-4-formyl-1H-pyrazol-1-yl)piperidine-1-carboxylate (50 mg, 0.17 mmol) in anhydrous methanol (1.5 mL) was added (1-diazo-2-oxo-propyl)-phosphonic acid dimethyl ester (40 mg, 0.21 mmol) and powdered potassium carbonate (48 mg, 0.35 mmol). The mixture was stirred at room temperature for 3.5 hours and then was quenched by the addition of excess saturated aqueous sodium bicarbonate solution. The layers were separated, and the aqueous layer extract two times with ethyl acetate. The organic extracts were combined and dried over sodium sulfate. The mixture was filtered, and the filtrate concentrated under reduced pressure to give an oil which was purified by chromatography on silica gel (10% to 100% ethyl acetate in heptane) to give isopropyl 4-(5-cyano-4-ethynyl-1H-pyrazol-1-yl)piperidine-1-carboxylate as a pale yellow solid (18 mg, 37%). ¹H NMR (400 MHz, deuterochloroform) delta 7.68 (s, 1H), 4.96-4.94 (m, 1H) 4.52-4.48 (m, 1H), 4.34 (m, 2H), 3.34 (s, 1H), 2.95-2.93 (m, 2H), 2.16-2.10 (m, 2H), 2.03-2.01 (m, 2H), 1.28 (d, 6H, J=6.3 Hz); LCMS (ES+): 287.5 (M+1).

### Step C: Isopropyl 4-(5-cyano-4-{[2-fluoro-4-(methylsulfonyl)phenyl]ethynyl}-1H-pyrazol-1-yl)piperidine-1-carboxylate

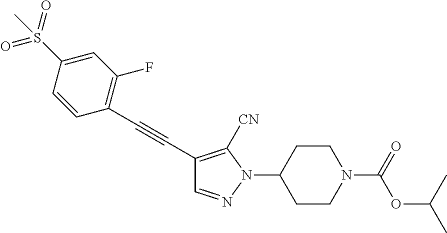

A solution of copper iodide (1.5 mg, 0.008 mmol), dichloro-bis(triphenylphosphine)palladium (II) (4.0 mg, 0.006 mmol), 1-bromo-2-fluoro-4-(methylsulfonyl)benzene (21 mg, 0.083 mmol) and triethylamine (0.028 mL, 0.19 mmol) in degassed N,N-dimethylformamide (0.5 mL) was added to a flask containing isopropyl 4-(5-cyano-4-ethynyl-1H-pyrazol-1-yl)piperidine-1-carboxylate (18 mg, 0.063 mmol) in degassed N,N-dimethylformamide (1.0 mL). The flask containing the initial solution was washed with degassed N,N-dimethylformamide (0.5 mL) which was then added to the reaction. The yellow solution was heated at 90° C. for 1.5 hours and then stirred at room temperature for 15 hours. The reaction was partitioned between water and ethyl acetate, and the layers were separated. The aqueous layer was extracted with ethyl acetate, and the organic extracts were combined and washed sequentially with water and brine and then dried over sodium sulfate. The mixture was filtered, and the filtrate concentrated under reduced pressure to an amber oil which was purified by chromatography on silica gel (10-90% ethyl acetate in heptane) to give isopropyl 4-(5-cyano-4-{[2-fluoro-4-(methylsulfonyl)phenyl]ethynyl}-1H-pyrazol-1-yl)piperidine-1-carboxylate as a pale yellow solid (20 mg, 69%). ¹H NMR (400 MHz, deuterochloroform) delta 7.75 (s, 1H), 7.73-7.68 (m, 3H), 4.94-4.91 (m, 1H) 4.52-4.48 (m, 1H), 4.33 (m, 2H), 3.07 (s, 3H), 2.97-2.91 (m, 2H), 2.16-2.09 (m, 2H), 2.09-2.01 (m, 2H), 1.25 (d, 6H, J=6.3 Hz); LCMS (ES+): 459.0 (M+1).

### Step D: Isopropyl 4-(5-cyano-4-{2-[2-fluoro-4-(methylsulfonyl)phenyl]ethyl}-1H-pyrazol-1-yl)piperidine-1-carboxylate

Isopropyl 4-(5-cyano-4-{[2-fluoro-4-(methylsulfonyl)phenyl]ethynyl}-1H-pyrazol-1-yl)piperidine-1-carboxylate (15 mg, 0.033 mmol) was dissolved in ethyl acetate (5.0 mL) and hydrogenated on a H-Cube flow reactor (ThalesNano, U.K.) at the full hydrogen setting with a flow rate of 1 mL/minute through a 10% Pd/C cartridge. The collected product in ethyl acetate was concentrated under reduced pressure to give isopropyl 4-(5-cyano-4-{2-[2-fluoro-4-(methylsulfonyl)phenyl]ethyl}-1H-pyrazol-1-yl)piperidine-1-carboxylate as a highly-pure, white solid (14 mg, 91%). ¹H NMR (400 MHz, deuterochloroform) delta 7.69-7.63 (m, 2H), 7.39-7.34 (m, 1H), 4.98-4.93 (m, 1H) 4.48-4.41 (m, 1H), 4.33 (m, 2H), 3.08 (s, 3H), 3.06-3.03 (t, 2H, J=7.6 Hz), 2.96-2.93 (m, 4H), 2.16-2.10 (m, 2H), 2.08-1.98 (m, 2H), 1.28 (d, 6H, J=6.3 Hz); LCMS (ES+): 463.1 (M+1).

### Example 10

### Isopropyl 4-[5-cyano-4-({[2-fluoro-4-(methylsulfonyl)phenyl]amino}methyl)-1H-pyrazol-1-yl]piperidine-1-carboxylate

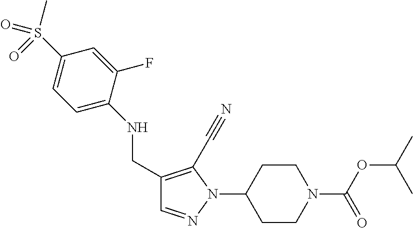

To a stirred solution of isopropyl 4-(5-cyano-4-formyl-1H-pyrazol-1-yl)piperidine-1-carboxylate (Example 9, Step A) (43 mg, 0.15 mmol) in 1.5 mL dichloroethane was added 2-fluoro-4-(methylthio)aniline (24 mg, 0.15 mmol) followed by 0.01 mL of acetic acid. The mixture was stirred at room temperature for 1.5 hours under a nitrogen atmosphere before adding sodium triacetoxy borohydride (52 mg, 0.24 mmol). After 115 hours the reaction mixture was diluted with dichloromethane and saturated aqueous sodium bicarbonate. The layers were separated and the aqueous layer was extracted twice with dichloromethane. The combined organic extracts were washed with brine, dried over magnesium sulfate, filtered, and the filtrate was concentrated in vacuo. The residue was purified by flash chromatography, eluting with a gradient mixture of ethyl acetate to heptane (0 to 50% ethyl acetate) to give 45 mg of the intermediate sulfide. Part of this material (23 mg, 0.053 mmol) was dissolved in 1 mL of dichloromethane and meta-chloroperbenzoic acid (36 mg, 0.16 mmol) was added in one portion. The mixture was stirred at room temperature for 2.5 hours before it was diluted with dichloromethane and saturated aqueous sodium carbonate solution. The organic layer was separated and was washed with saturated aqueous sodium carbonate solution, brine, dried over magnesium sulfate, filtered and the filtrate was concentrated in vacuo. The sample was purified by reversed-phase HPLC (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 90% water/10% acetonitrile linear to 0% water/100% acetonitrile in 8.5 minutes, hold at 0% water/100% acetonitrile to 10.0 minutes. Flow: 25 mL/minute. LCMS: (MS ES+: 464.2).

### Example 11

### Isopropyl 4-{5-cyano-4-[(2,4-difluorophenoxy)methyl]-1H-pyrazol-1-yl}piperidine-1-carboxylate

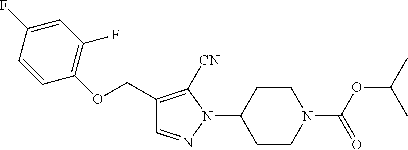

Isopropyl 4-(5-cyano-4-((methylsulfonyloxy)methyl)-1H-pyrazol-1-yl)piperidine-1-carboxylate (Preparation 10) (166.5 mg, 0.449 mmol), 2,4-difluorophenol (0.052 mL, 0.539 mmol), and cesium carbonate (293 mg, 0.898 mmol) were placed in microwave vial, dissolved in acetonitrile (3 mL), and heated in a microwave reactor at 110 degrees Celsius for 20 minutes. The mixture was cooled to room temperature and concentrated under vacuum, diluted with 1 N sodium hydroxide solution, and extracted three times with dichloromethane. The combined organic extracts were washed with brine, dried over sodium sulfate, filtered, and the filtrate was concentrated under vacuum. The crude material was purified by preparative reverse-phase HPLC on a Waters Atlantis C₁₈ column 4.6×50 mm, 0.005 mm eluting with a gradient of water in acetonitrile (0.05% trifluoroacetic acid modifier) to give isopropyl 4-{5-cyano-4-[(2,4-difluorophenoxy)methyl]-1H-pyrazol-1-yl}piperidine-1-carboxylate. Analytical LCMS: retention time: 3.62 minutes (Waters Atlantis C₁₈ 4.6×50 mm, 0.005 mm; 95% water/acetonitrile linear gradient to 5% water/acetonitrile over 4.0 min; 0.05% trifluoroacetic acid modifier; flow rate 2.0 mL/minute); LCMS (ES+): 405.18 (M+H).

### Example 12

### Isopropyl 4-{5-cyano-4-[(2-methylphenoxy)methyl]-1H-pyrazol-1-yl}piperidine-1-carboxylate

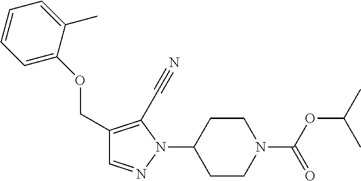

To a stirred solution of ortho-cresol (21 mg, 0.19 mmol) and isopropyl 4-(5-cyano-4-((methylsulfonyloxy)methyl)-1H-pyrazol-1-yl)piperidine-1-carboxylate (Preparation 10) (60 mg, 0.16 mmol) in acetonitrile (1.6 mL) was added cesium carbonate (106 mg, 0.32 mmol). The mixture was heated at reflux for 15 hours. After cooling to room temperature the crude material was concentrated to dryness in vacuo, and the residue was taken up in water and extracted 3 times with ethyl acetate (20 mL each extraction). The combined organic extracts were washed with brine, dried over sodium sulfate, filtered and the filtrate was concentrated to dryness under vacuum to give a tan residue (0.065 g, 100%). The crude sample was dissolved in dimethyl sulfoxide (1 mL) and purified by preparative reverse phase HPLC on a Waters Sunfire C₁₈ 19×100 mm, 0.005 mm column, eluting with a linear gradient of 80% water/acetonitrile to 0% water/acetonitrile in 8.5 minutes, followed by a 1.5 minute period at 0% water/acetonitrile (0.05% trifluoroacetic acid modifier); flow rate: 25 mL/minute. Analytical LCMS: retention time 3.82 minutes (Waters Atlantis C₁₈ 4.6×50 mm, 0.005 mm column; 95% water/acetonitrile linear gradient to 5% water/acetonitrile over 4.0 minutes, followed by a 1 minute period at 5% water/acetonitrile; 0.05% trifluoroacetic acid modifier; flow rate: 2.0 mL/minute); LCMS (ES+) 383.2 (M+1).

### Example 13

### 1-Methylcyclopropyl 4-{5-cyano-4-[(2,5-difluorophenoxy)methyl]-1H-pyrazol-1-yl}piperidine-1-carboxylate

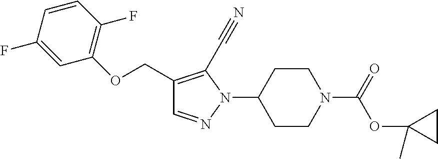

### A) tert-butyl 4(5-cyano-4-((2,5-difluorophenoxy)methyl)-1H-pyrazol-1-yl)piperidine-1-carboxylate

To a stirred solution of 2,5-difluorophenol (54 mg, 0.39 mmol) and tert-butyl 4-(5-cyano-4-((methylsulfonyloxy)methyl)-1H-pyrazol-1-yl)piperidine-1-carboxylate (Preparation 16) (126 mg, 0.33 mmol) in 3 mL of acetonitrile was added cesium carbonate (214 mg, 0.66 mmol). The mixture was heated at reflux for 15 hours. The mixture was cooled to room temperature and diluted with ethyl acetate and water. The layers were separated and the aqueous phase was extracted with ethyl acetate. The combined organic phases were washed with brine, dried over magnesium sulfate, filtered, and the filtrate was concentrated in vacuo to give tert-butyl 4-(5-cyano-4-((2,5-difluorophenoxy)methyl)-1H-pyrazol-1-yl)piperidine-1-carboxylate which was used in the next step without purification.

### B) 4((2,5-Difluorophenoxy)methyl)-1-(piperidin-4-yl)-1H-pyrazole-5-carbonitrile

To a solution of tert-butyl 4-(5-cyano-4-((2,5-difluorophenoxy)methyl)-1H-pyrazol-1-yl)piperidine-1-carboxylate (137 mg, 0.33 mmol) in 5 mL of dichloromethane was added 0.82 mL of hydrochloric acid (4 M in 1,4-dioxane). The mixture was stirred at room temperature for 2 hours before the mixture was concentrated in vacuo to give 4-((2,5-difluorophenoxy)methyl)-1-(piperidin-4-yl)-1H-pyrazole-5-carbonitrile which was used in the next step without purification.

### C) 1-Methylcyclopropyl 4-{5-cyano-4-[(2,5-difluorophenoxy)methyl]-1H-pyrazol-1-yl}piperidine-1-carboxylate

To a stirred solution of 4-((2,5-difluorophenoxy)methyl)-1-(piperidin-4-yl)-1H-pyrazole-5-carbonitrile (104 mg, 0.33 mmol) in 3.3 mL of dichloromethane was added triethylamine (0.18 mL, 1.3 mmol) followed by 1-methylcyclopropyl 4-nitrophenyl carbonate (see Preparation 26 and WO09105717) (171 mg, 0.72 mmol) at room temperature. The resulting bright yellow mixture was stirred for 15 hours under a nitrogen atmosphere. The reaction mixture was diluted with dichloromethane and water. The layers were separated and the aqueous phase was extracted with dichloromethane. The combined organic phases were washed with saturated aqueous sodium bicarbonate, brine, dried over magnesium sulfate, filtered and the filtrate was concentrated in vacuo to give 225 mg of crude material. Part (45 mg) of this material was dissolved in dimethyl sulfoxide (0.9 mL) and purified by preparative reverse-phase HPLC on a Waters XBridge C₁₈ column 19×100 mm, 0.005 column eluting with a gradient of water in acetonitrile (0.03% ammonium hydroxide modifier). Analytical LCMS: retention time 3.60 minutes (Atlantis C₁₈ 4.6×50 mm, 5 micrometer column; 95% water/acetonitrile linear gradient to 5% water/acetonitrile over 4 minutes; 0.05% trifluoroacetic modifier; flow rate 2.0 mL/minute; LCMS (ES+): 417.1 (M+H).

### Example 14

### 1-Methylcyclopropyl 4-{5-cyano-4-[(2,3-difluorophenoxy)methyl]-1H-pyrazol-1-yl}piperidine-1-carboxylate

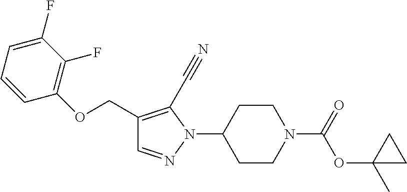

The title compound was prepared using commercially available 2,3-diflurophenol, following procedures analogous to Example 13. The crude material (49 mg) was dissolved in dimethyl sulfoxide (0.9 mL) and purified by preparative reverse-phase HPLC on a Waters XBridge C₁₈ column 19×100 mm, 0.005 column eluting with a gradient of water in acetonitrile (0.03% ammonium hydroxide modifier). Analytical LCMS: retention time 3.62 minutes (Atlantis C₁₈ 4.6×50 mm, 5 micrometer column; 95% water/acetonitrile linear gradient to 5% water/acetonitrile over 4 minutes; 0.05% trifluoroacetic modifier; flow rate 2.0 mL/minute; LCMS (ES+): 417.2 (M+H).

### Example 15

### 1-Methylcyclopropyl 4-{4-[(4-carbamoyl-2-fluorophenoxy)methyl]-5-cyano-1H-pyrazol-1-yl}piperidine-1-carboxylate

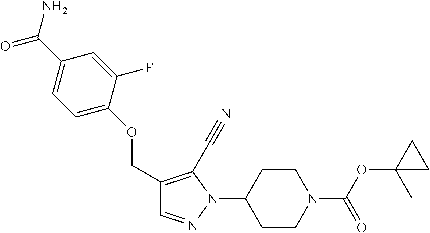

### A) tert-Butyl 4-(4-((4-carbamoyl-2-fluorophenoxy)methyl)-5-cyano-1H-pyrazol-1-yl)piperidine-1-carboxylate

To a stirred solution of tert-butyl 4-(5-cyano-4-(hydroxymethyl)-1H-pyrazol-1-yl)piperidine-1-carboxylate (Preparation 15) (200 mg, 0.65 mmol), 3-fluoro-4-hydroxybenzamide (Preparation 23) (100 mg, 0.64 mmol) and triphenylphosphine (188 mg, 0.72 mmol) in 3 mL of 1,4-dioxane was added drop-wise diethyl azodicarboxylate (0.11 mL, 0.69 mmol). The resulting mixture was stirred overnight at room temperature before the mixture was concentrated in vacuo. The residue was purified by flash chromatography, eluting with a gradient of 30 to 70% ethyl acetate in heptane to give tert-butyl 4-(4-((4-carbamoyl-2-fluorophenoxy)methyl)-5-cyano-1H-pyrazol-1-yl)piperidine-1-carboxylate as a white solid (215 mg).

### B) 4-((5-Cyano-1-(piperidin-4-yl)-1H-pyrazol-4-yl)methoxy)-3-fluorobenzamide

To a stirred solution of tent-butyl 4-(4-((4-carbamoyl-2-fluorophenoxy)methyl)-5-cyano-1H-pyrazol-1-yl)piperidine-1-carboxylate (215 mg, 0.48 mmol) in 2 mL of dichloromethane was added 1 mL of trifluoroacetic acid at room temperature. After 1 hour the solution was concentrated in vacuo. The residue was purified by flash chromatography, eluting with a gradient mixture of 1 to 15% of methanol in dichloromethane containing 2% of aqueous ammonia) to give 4-((5-cyano-1-(piperidin-4-yl)-1H-pyrazol-4-yl)methoxy)-3-fluorobenzamide as a white solid (150 mg).

### C) 1-Methylcyclopropyl 4-{4-[(4-carbamoyl-2-fluorophenoxy)methyl]-5-cyano-1H-pyrazol-1-yl}piperidine-1-carboxylate

To a stirred solution of 4-((5-cyano-1-(piperidin-4-yl)-1H-pyrazol-4-yl)methoxy)-3-fluorobenzamide (40 mg, 0.12 mmol) in 1 mL of dichloromethane was added triethylamine (0.036 mL, 0.26 mmol), followed by 1-methylcyclopropyl 4-nitrophenyl carbonate (Preparation 26 and WO09105717) (60 mg, 0.26 mmol) at room temperature. The resulting bright yellow mixture was stirred for 2 hours under a nitrogen atmosphere at 65 degrees Celsius. The reaction was cooled to room temperature, diluted with water and extracted twice with dichloromethane. The combined organic extracts were washed with saturated sodium bicarbonate, dried over sodium sulfate, filtered and the filtrate was concentrated in vacuo. The residue was purified by flash chromatography, eluting with a gradient of 40 to 90% ethyl acetate in heptane to give 1-methylcyclopropyl 4-{4-[(4-carbamoyl-2-fluorophenoxy)methyl]-5-cyano-1H-pyrazol-1-yl}piperidine-1-carboxylate as white solid (34 mg). ¹H NMR (400 MHz, deuterochloroform) delta 0.59-0.67 (m, 2H), 0.83-0.92 (m, 2H), 1.54 (s, 3H), 2.02 (d, J=4.10 Hz, 2H), 2.04-2.22 (m, 2H), 2.91 (br. s., 2H), 4.11-4.43 (m, 2H), 4.44-4.55 (m, 1H), 5.15 (s, 2H), 7.03-7.10 (m, 1H), 7.52-7.62 (m, 2H), 7.68 (s, 1H). 1H NMR indicated the presence of less than 10% of what is believed to be the corresponding isopropyl carbamate derivative (from the isopropyl 4-nitrophenyl carbonate contaminating the 1-methylcyclopropyl 4-nitrophenyl carbonate). LCMS (ES) 442.4 (M+1).

### Example 16

### 1-Methylcyclopropyl 4-{4-[(4-carbamoylphenoxy)methyl]-5-cyano-1H-pyrazol-1-yl}piperidine-1-carboxylate

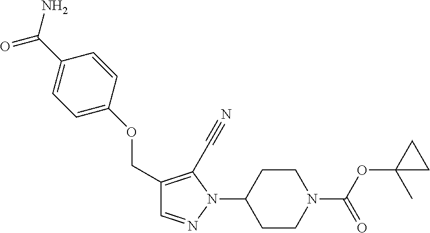

The title compound was prepared using commercially available 4-hydroxybenzamide, following procedures analogous to Example 15. ¹H NMR (400 MHz, deuterochloroform) delta 0.57-0.67 (m, 2H), 0.84-0.91 (m, 2H), 1.56 (s, 3H), 1.93-2.05 (m, 2H), 2.05-2.19 (m, 2H), 2.91 (t, J=15.62 Hz, 2H), 4.26 (br. s., 2H), 4.44-4.55 (m, 1H), 5.09 (s, 2H), 6.96-7.04 (m, 2H), 7.66 (s, 1H), 7.75-7.82 (m, 2H). ¹H NMR indicated the presence of less than 10% of what is believed to be the corresponding isopropyl carbamate derivative (from the isopropyl 4-nitrophenyl carbonate contaminating the 1-methylcyclopropyl 4-nitrophenyl carbonate). LCMS (ES) 424.4 (M+1).

### Example 17

### 1-Methylcyclopropyl 4-(5-cyano-4-((4-cyanophenoxy)methyl)-1H-pyrazol-1-yl)piperidine-1-carboxylate

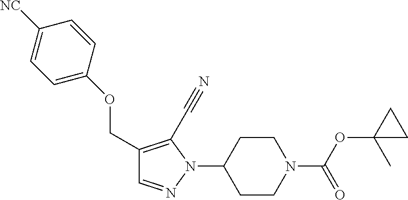

The title compound was prepared using commercially available 4-hydroxybenzonitrile, following procedures analogous to Example 15. The purification of the crude reaction mixture was performed by flash chromatography, eluting with a gradient mixture of ethyl acetate in heptane (0 to 100% ethyl acetate). 1H NMR (500 MHz, deuterochloroform) delta 0.60-0.70 (m, 2H), 0.84,-0.94 (m, 2H), 1.23-1.31 (m, 1H), 1.56 (s, 3H), 2.01-2.15 (m, 4H), 2.93 (m, 2H), 4.11-4.37 (m, 1H), 4.49-4.55 (m, 1H), 5.10 (s, 2H), 7.03 (d, J=8.78 Hz, 2H), 7.63 (d, J=8.78 Hz, 2H), 7.67 (s, 1H).

### Example 18

### Isopropyl 4-(4-((4-(1H-pyrazol-1-yl)phenoxy)methyl)-5-cyano-1H-pyrazol-1-yl)piperidine-1-carboxylate

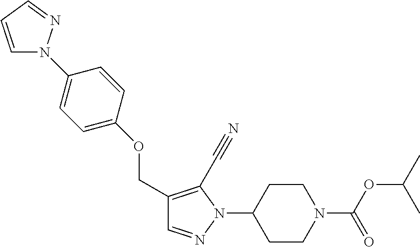

The title compound was prepared using 4-(1H-pyrazol-1-yl)phenol (WO 2003072547), following a procedure analogous to Example 12. The purification of the crude reaction mixture was performed by flash chromatography, eluting with a gradient mixture of ethyl acetate in heptane (0 to 100% ethyl acetate). 1H NMR (500 MHz, deuterochloroform) delta 1.28 (d, J=6.34 Hz, 6H), 2.01-2.09 (m, 2H), 2.17 (m, 2H), 2.91-2.99 (m, 2H), 4.37 (m, 2H), 4.50-4.58 (m, 1H), 4.93-4.98 (m, 1H), 5.11 (s, 2H), 6.47 (t, J=2.07 Hz, 1H), 7.07 (d, J=9.03 Hz, 2H), 7.64 (d, J=9.03 Hz, 2H), 7.70 (s, 1H), 7.72 (d, J=1.71 Hz, 1H), 7.86 (d, J=2.44 Hz, 1H). LCMS (ES) 435.4(M+1).

### Example 19

### Isopropyl 4-(5-cyano-4-((2-fluoro-4-(1H-tetrazol-5-yl)phenoxy)methyl)-1H-pyrazol-1-yl)piperidine-1-carboxylate and Isopropyl 4-(5-cyano-4-((2-fluoro-4-(2H-tetrazol-5-yl)phenoxy)methyl)-1H-pyrazol-1-yl)piperidine-1-carboxylate

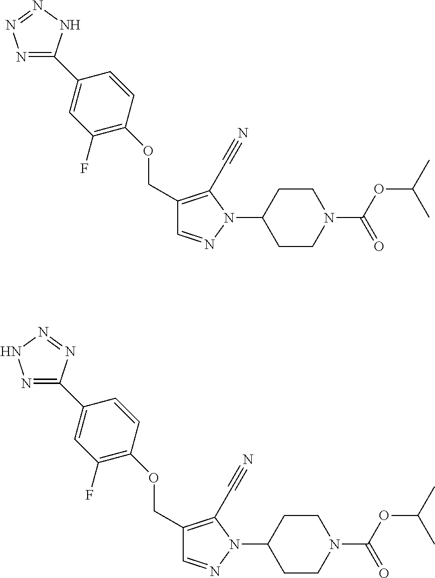

### A) Isopropyl 4-(5-cyano-4-((2-fluoro-4-(1-((2-(trimethylsilyl)ethoxy)methyl)-1H-tetrazol-5-yl)phenoxy)methyl)-1H-pyrazol-1-yl)piperidine-1-carboxylate and Isopropyl 4-(5-cyano-4-((2-fluoro-4-(2-((2-(trimethylsilyl)ethoxy)methyl)-2H-tetrazol-5-yl)phenoxy)methyl)-1H-pyrazol-1-yl)piperidine-1-carboxylate

To a stirred solution of isopropyl 4-(5-cyano-4-(hydroxymethyl)-1H-pyrazol-1-yl)piperidine-1-carboxylate (94 mg, 0.322 mmol), 2-fluoro-4-(1-((2-(trimethylsilyl)ethoxy)methyl)-1H-tetrazol-5-yl)phenol and 2-fluoro-4-(2-((2-(trimethylsilyl)ethoxy)methyl)-2H-tetrazol-5-yl)phenol (Preparation 17) (100 mg, 0.322 mmol) and triphenylphosphine (110 mg, 0.42 mmol) in 5 mL of 1,4-dioxane was added drop-wise diethyl azodicarboxylate (0.060 mL, 0.39 mmol). The resulting mixture was stirred overnight at room temperature before the mixture was concentrated in vacuo. The residue was purified by flash chromatography, eluting with a gradient of 10 to 40% ethyl acetate in heptane to give isopropyl 4-(5-cyano-4-((2-fluoro-4-(1-((2-(trimethylsilyl)ethoxy)methyl)-1H-tetrazol-5-yl)phenoxy)methyl)-1H-pyrazol-1-yl)piperidine-1-carboxylate and isopropyl 4-(5-cyano-4-((2-fluoro-4-(2-((2-(trimethylsilyl)ethoxy)methyl)-2H-tetrazol-5-yl)phenoxy)methyl)-1H-pyrazol-1-yl)piperidine-1-carboxylate (140 mg, 74% yield).

Isopropyl 4-(5-cyano-4-((2-fluoro-4-(2-((2-(trimethylsilyl)ethoxy)methyl)-2H-tetrazol-5-yl)phenoxy)methyl)-1H-pyrazol-1-yl)piperidine-1-carboxylate. ¹H NMR (400 MHz, deuterochloroform) delta −0.05-0.01 (m, 9H), 0.90-1.00 (m, 2H), 1.18-1.27 (m, 6H), 2.02 (br. s., 2H), 2.13 (m, 2H) 2.93 (br. s., 2H), 3.65-3.78 (m, 2H), 4.30 (d, J=7.22 Hz, 2H), 4.46-4.58 (m, 1H), 4.86-4.98 (m, 1H), 5.16 (s, 2H), 5.89 (s, 2H), 7.09-7.18 (m, 1H), 7.69 (s, 1H), 7.88-7.96 (m, 2H). LCMS (ES) 585.1 (M+1).

### B) Isopropyl 4-(5-cyano-4-((2-fluoro-4-(1H-tetrazol-5-yl)phenoxy)methyl)-1H-pyrazol-1-yl)piperidine-1-carboxylate and Isopropyl 4-(5-cyano-4-((2-fluoro-4-(2H-tetrazol-5-yl)phenoxy)methyl)-1H-pyrazol-1-yl)piperidine-1-carboxylate

Isopropyl 4-(5-cyano-4-((2-fluoro-4-(1-((2-(trimethylsilyl)ethoxy)methyl)-1H-tetrazol-5-yl)phenoxy)methyl)-1H-pyrazol-1-yl)piperidine-1-carboxylate and isopropyl 4-(5-cyano-4-((2-fluoro-4-(2-((2-(trimethylsilyl)ethoxy)methyl)-2H-tetrazol-5-yl)phenoxy)methyl)-1H-pyrazol-1-yl)piperidine-1-carboxylate (220 mg, 0.38 mmol) were dissolved in ethanol (3 mL) and a solution of aqueous 2 M hydrochloric acid (3 mL) was added drop-wise, The resulting mixture was stirred at 50 degrees Celsius for 4 hours before being cooled down to room temperature and filtered. The resulting white solid was washed with ethyl acetate and heptane (1/1 volume) and dried under reduced pressure to give the title compound (80 mg, 47% yield). ¹H NMR (400 MHz, deutero dimethyl sulfoxide) delta 1.16 (d, J=6.25 Hz, 6H), 1.76-1.90 (m, 2H), 1.98 (dd, J=14.45, 3.12 Hz, 2H), 2.99 (br. s., 2H), 4.04 (d, J=15.81 Hz, 2H), 4.59-4.71 (m, 1H), 4.70-4.82 (m, 1H), 5.27 (s, 2H), 7.47-7.57 (m, 1H), 7.80-7.83 (m, 1H), 7.83-7.87 (m, 1H), 7.90 (s, 1H). LCMS (ES) 455.0 (M+1).

### Example 20

### Isopropyl 4-(5-cyano-4-((2-fluoro-4-(1-methyl-1H-tetrazol-5-yl)phenoxy)methyl)-1H-pyrazol-1-yl)piperidine-1-carboxylate and

### Example 21

### Isopropyl 4-(5-cyano-4-((2-fluoro-4-(2-methyl-2H-tetrazol-5-yl)phenoxy)methyl)-1H-pyrazol-1-yl)piperidine-1-carboxylate

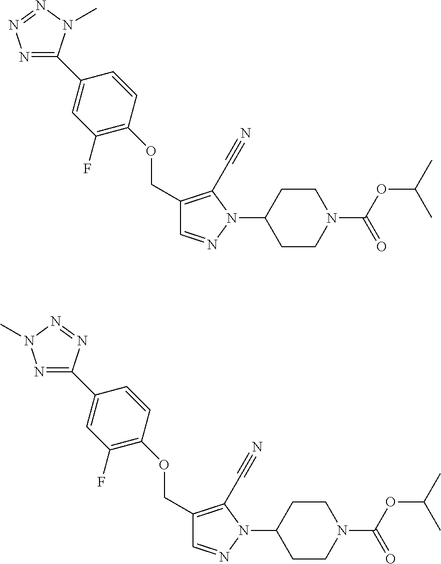

To a solution of isopropyl 4-(5-cyano-4-((2-fluoro-4-(1H-tetrazol-5-yl)phenoxy)methyl)-1H-pyrazol-1-yl)piperidine-1-carboxylate and isopropyl 4-(5-cyano-4-((2-fluoro-4-(2H-tetrazol-5-yl)phenoxy)methyl)-1H-pyrazol-1-yl)piperidine-1-carboxylate (70 mg, 0.15 mmol) at room temperature in tetrahydrofuran (2 mL) was added sodium hydride (14 mg, 0.31 mmol) in two portions, and the resulting mixture was stirred for 5 minutes. Iodomethane (0.03 mL, 0.46 mmol) was then added and the reaction mixture was stirred at room temperature for an additional 16 hours. The reaction was quenched by addition of water and the mixture was diluted with ethyl acetate. The organic phase was separated and the aqueous phase was extracted twice with ethyl acetate. The combined organic extracts were washed with brine, dried over magnesium sulfate, filtered and the filtrate was concentrated in vacuo. The residue was purified by flash silica gel chromatography, eluting with a gradient mixture of ethyl acetate in heptane (30 to 60% ethyl acetate) to give isopropyl 4-(5-cyano-4-((2-fluoro-4-(1-methyl-1H-tetrazol-5-yl)phenoxy)methyl)-1H-pyrazol-1-yl)piperidine-1-carboxylate (10 mg, 14% yield) and isopropyl 4-(5-cyano-4-((2-fluoro-4-(2-methyl-2H-tetrazol-5-yl)phenoxy)methyl)-1H-pyrazol-1-yl)piperidine-1-carboxylate (30 mg, 42% yield).

Isopropyl 4-(5-cyano-4-((2-fluoro-4-(1-methyl-1H-tetrazol-5-yl)phenoxy)methyl)-1H-pyrazol-1-yl)piperidine-1-carboxylate (Example 20). ¹H NMR (400 MHz, deuterochloroform) delta 1.18-1.28 (m, 6H), 1.95-2.06 (m, 2H), 2.13 (m, 2H), 2.85-3.02 (m, 2H), 4.17 (s, 3H), 4.36 (d, J=10.15 Hz, 2H), 4.46-4.57 (m, 1H) 4.92 (spt, 1H), 5.19 (s, 2H), 7.17-7.24 (m, 1H), 7.48-7.58 (m, 2H), 7.70 (s, 1H). LCMS (ES) 469.0 (M+1).

Isopropyl 4-(5-cyano-4-((2-fluoro-4-(2-methyl-2H-tetrazol-5-yl)phenoxy)methyl)-1H-pyrazol-1-yl)piperidine-1-carboxylate (Example 21). ¹H NMR (400 MHz, deuterochloroform) delta 1.24 (d, J=6.25 Hz, 6H) 1.95-2.05 (m, 2H) 2.13 (m, 2H) 2.93 (t, J=12.59 Hz, 2H) 4.31 (br. s., 2H) 4.37 (s, 3H) 4.51 (m, 1H) 4.92 (m, 1H) 5.16 (s, 2H) 7.09-7.16 (m, 1H) 7.69 (s, 1H) 7.83-7.87 (m, 1H) 7.87-7.90 (m, 1H). LCMS (ES) 469.0 (M+1).

### Example 22

### Isopropyl 4-(5-cyano-4-((2-fluoro-4-(2-(2-hydroxyethyl)-2H-tetrazol-5-yl)phenoxy)methyl)-1H-pyrazol-1-yl)piperidine-1-carboxylate

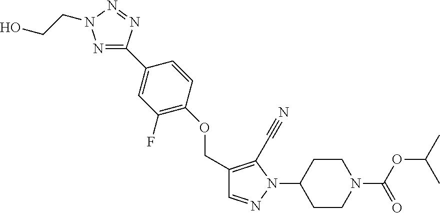

### A) Isopropyl 4-(5-cyano-4-((2-fluoro-4-(2-(2-(trimethylsilyloxy)ethyl)-2H-tetrazol-5-yl)phenoxy)methyl)-1H-pyrazol-1-yl)piperidine-1-carboxylate

To a stirred solution of isopropyl 4-(5-cyano-4-(hydroxymethyl)-1H-pyrazol-1-yl)piperidine-1-carboxylate (Preparation 5) (78 mg, 0.266 mmol), 2-fluoro-4-(2-(2-(trimethylsilyloxy)ethyl)-2H-tetrazol-5-yl)phenol (Preparation 19) (90 mg, 0.27 mmol) and triphenylphosphine (77 mg, 0.29 mmol) in 5 mL of 1,4-dioxane was added drop-wise diethyl azodicarboxylate (0.046 mL, 0.28 mmol). The resulting mixture was stirred for 15 hours at room temperature before the mixture was concentrated in vacuo. The residue was purified by flash chromatography, eluting with a gradient of 5 to 40% ethyl acetate in heptane to give isopropyl 4-(5-cyano-4-((2-fluoro-4-(2-(2-(trimethylsilyloxy)ethyl)-2H-tetrazol-5-yl)phenoxy)methyl)-1H-pyrazol-1-yl)piperidine-1-carboxylate (140 mg, 86% yield).

### B) Isopropyl 4-(5-cyano-4-((2-fluoro-4-(2-(2-hydroxyethyl)-2H-tetrazol-5-yl)phenoxy)methyl)-1H-pyrazol-1-yl)piperidine-1-carboxylate

Isopropyl 4-(5-cyano-4-((2-fluoro-4-(2-(2-(trimethylsilyloxy)ethyl)-2H-tetrazol-5-yl)phenoxy)methyl)-1H-pyrazol-1-yl)piperidine-1-carboxylate (140 mg, 0.228 mmol) was dissolved in methanol (2 mL), and a solution of 4 M hydrochloric acid (1 mL) in 1,4-dioxane was added drop-wise, The resulting mixture was stirred at room temperature for 2 hours before being concentrated under reduced pressure. The residue (160 mg) was divided and ca. 50 mg of the crude was purified by reverse-phase HPLC to give the title compound (30 mg, 26%) (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/min. Detection: 215 nm. LCMS (ES+): 499.5 (M+1).

### Example 23

### Isopropyl 4-(5-cyano-4-((2-fluoro-4-(1-(2-hydroxyethyl)-1H-tetrazol-5-yl)phenoxy)methyl)-1H-pyrazol-1-yl)piperidine-1-carboxylate

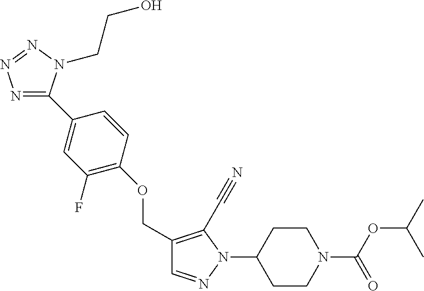

### A) Isopropyl 4-(5-cyano-4-((2-fluoro-4-(1-(2-(trimethylsilyloxy)ethyl)-1H-tetrazol-5-yl)phenoxy)methyl)-1H-pyrazol-1-yl)piperidine-1-carboxylate

To a stirred solution of isopropyl 4-(5-cyano-4-(hydroxymethyl)-1H-pyrazol-1-yl)piperidine-1-carboxylate (43 mg, 0.15 mmol), 2-fluoro-4-(1-(2-(trimethylsilyloxy)ethyl)-1H-tetrazol-5-yl)phenol (preparation 20) (50 mg, 0.15 mmol) and triphenylphosphine (43 mg, 0.16 mmol) in 3 mL of 1,4-dioxane was added drop-wise diethyl azodicarboxylate (0.025 mL, 0.16 mmol). The resulting mixture was stirred overnight at room temperature before the mixture was concentrated in vacuo. The residue was purified by flash chromatography, eluting with a gradient of 30 to 70% ethyl acetate in heptane to give isopropyl 4-(5-cyano-4-((2-fluoro-4-(1-(2-(trimethylsilyloxy)ethyl)-1H-tetrazol-5-yl)phenoxy)methyl)-1H-pyrazol-1-yl)piperidine-1-carboxylate (50 mg, 55% yield).

### B) Isopropyl 4-(5-cyano-4-((2-fluoro-4-(1-(2-hydroxyethyl)-1H-tetrazol-5-yl)phenoxy)methyl)-1H-pyrazol-1-yl)piperidine-1-carboxylate

Isopropyl 4-(5-cyano-4-((2-fluoro-4-(1-(2-(trimethylsilyloxy)ethyl)-1H-tetrazol-5-yl)phenoxy)methyl)-1H-pyrazol-1-yl)piperidine-1-carboxylate (50 mg, 0.082 mmol) was dissolved in methanol (2 mL) and a solution of 4 M hydrochloric acid (1 mL) in 1,4-dioxane was added drop-wise, The resulting mixture was stirred at room temperature for 2 hours before being concentrated under reduced pressure. The residue (60 mg) was purified by reversed-phase HPLC to give the title compound (20 mg, 49% yield) (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 80% water/20% acetonitrile linear to 0% water/100% acetonitrile in 8.5 minutes, hold at 0% water/100% acetonitrile to 10.0 minutes. Flow: 25 mL/min. Detection: 215 nm LCMS (ES+): 499.4 (M+1).

### Example 24

### 1-Methylcyclopropyl 4-(5-cyano-4-{[2-fluoro-4-(1-methyl-1H-tetrazol-5-yl)phenoxy]methyl}-1H-pyrazol-1-yl)piperidine-1-carboxylate

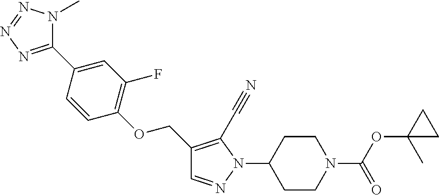

The title compound was prepared using 2-fluoro-4-(1-methyl-1H-tetrazol-5-yl)phenol (Preparation 21), following procedures analogous to Example 15. ¹H NMR (400 MHz, deuterochloroform) delta 0.58-0.67 (m, 2H), 0.83-0.92 (m, 2H), 1.57 (s, 3H), 1.94-2.05 (m, 2H), 2.05-2.21 (m, 2H), 2.92 (t, J=12.98 Hz, 2H), 4.17 (s, 3H), 4.32 (br. s., 2H), 4.43-4.56 (m, 1H), 5.19 (s, 2H), 7.17-7.24 (m, 1H), 7.48-7.58 (m, 2H), 7.70 (s, 1H). 1H NMR indicated the presence of less than 10% of what is believed to be the corresponding isopropyl carbamate derivative (from the isopropyl 4-nitrophenyl carbonate contaminating the 1-methylcyclopropyl 4-nitrophenyl carbonate). LCMS (ES) 481.6 (M+1).

### Example 25

### 1-Methylcyclopropyl 4-(5-cyano-4-{[4-(1-methyl-1H-tetrazol-5-yl)phenoxy]methyl}-1H-pyrazol-1-yl)piperidine-1-carboxylate

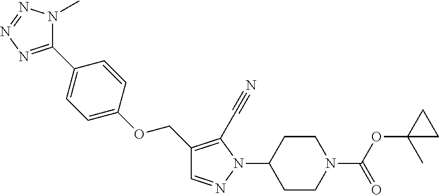

The title compound was prepared using 4-(1-methyl-1H-tetrazol-5-yl)phenol (Preparation 22), following procedures analogous to Example 15.

¹H NMR (400 MHz, deuterochloroform) delta 0.60-0.67 (m, 2H), 0.83-0.91 (m, 2H), 1.58 (s, 3H), 1.96-2.06 (m, 2H), 2.06-2.21 (m, 2H), 2.84-3.00 (m, 2H), 4.16 (s, 3H), 4.33 (br. s., 2H), 4.45-4.57 (m, 1H), 5.12 (s, 2H), 7.10-7.15 (m, 2H), 7.68 (s, 1H), 7.69-7.74 (m, 2H). 1H NMR indicated the presence of less than 10% of what is believed to be the corresponding isopropyl carbamate derivative (from the isopropyl 4-nitrophenyl carbonate contaminating the 1-methylcyclopropyl 4-nitrophenyl carbonate). LCMS (ES) 463.5 (M+1).

### Example 26

### 1-Methylcyclopropyl 4-(4-((4-carbamoyl-3-fluorophenoxy)methyl)-5-cyano-1H-pyrazol-1-yl)piperidine-1-carboxylate

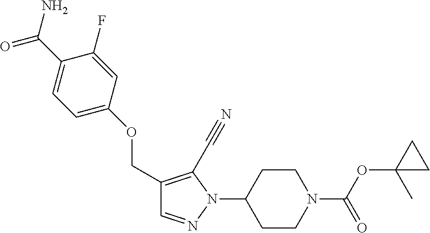

The title compound was prepared using 2-fluoro-4-hydroxybenzamide (Preparation 24), following procedures analogous to Example 13. ¹H NMR (400 MHz, deuterochloroform) delta 0.57-0.65 (m, 2H), 0.82-0.89 (m, 2H), 1.53 (s, 3H), 1.92-2.04 (m, 2H), 2.10 (qd, J=12.14, 4.20 Hz, 2H), 2.90 (br. s., 2H), 4.32 (br. s., 2H), 4.49 (tt, J=11.25, 4.37 Hz, 1H), 5.02-5.09 (m, 2H), 6.00 (br. s., 1H), 6.51-6.64 (m, 1H), 6.69 (dd, J=13.66, 2.54 Hz, 1H), 6.84 (dd, J=8.78, 2.54 Hz, 1H), 7.64 (s, 1H), 8.07 (t, J=9.08 Hz, 1H). 1H NMR indicated the presence of less than 10% of what is believed to be the corresponding isopropyl carbamate derivative (from the isopropyl 4-nitrophenyl carbonate contaminating the 1-methylcyclopropyl 4-nitrophenyl carbonate). LCMS (ES) 442.4 (M+1).

### Example 27

### Isopropyl 4-(5-cyano-4-{1-[2-fluoro-4-(methylsulfonyl)phenoxy]ethyl}-1H-pyrazol-1-yl)piperidine-1-carboxylate

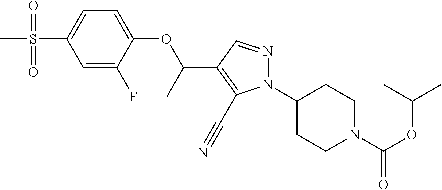

The title compound was prepared using 2-fluoro-4-(methylsulfonyl)phenol and isopropyl 4-(5-cyano-4-(1-hydroxyethyl)-1H-pyrazol-1-yl)piperidine-1-carboxylate (Preparation 25), following procedures analogous to Example 15. The sample was purified by reversed-phase HPLC (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 80% water/20% acetonitrile linear to 0% water/100% acetonitrile in 8.5 minutes, hold at 0% water/100% acetonitrile to 10.0 minutes. Flow: 25 mL/minute. LCMS (ES+): 479.2 M+1).

### Example 28

### Isopropyl 4-(5-cyano-4-{1-[(2-methylpyridin-3-yl)oxy]ethyl}-1H-pyrazol-1-yl)piperidine-1-carboxylate

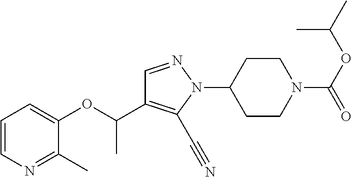

The title compound was prepared using 2-methylpyridin-3-ol and isopropyl 4-(5-cyano-4-(1-hydroxyethyl)-1H-pyrazol-1-yl)piperidine-1-carboxylate (Preparation 25), following procedures analogous to Example 15. The sample was purified by reversed-phase HPLC (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/minute. LCMS (ES+): 398.2 M+1).

### Example 29

### Isopropyl 4-(5-cyano-4-{2-[2-fluoro-4-(methylsulfonyl)phenyl]propyl}-1H-pyrazol-1-yl)piperidine-1-carboxylate

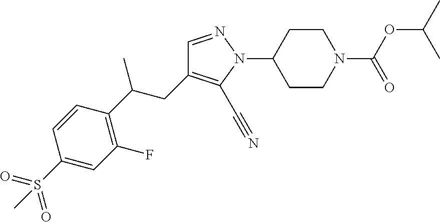

### A) Isopropyl 4-(5-cyano-4-vinyl-1H-pyrazol-1-yl)piperidine-1-carboxylate

To a stirred mixture of (methyl)-triphenylphosphonium bromide (323 mg, 0.88 mmol) in tetrahydrofuran (5 mL) at −78 degrees Celsius was added drop-wise n-butyllithium (0.360 mL, 0.89 mmol, 2.5 M in hexanes). The resulting yellow mixture was stirred at −78 degrees Celsius for 30 minutes, and then a solution of isopropyl 4-(5-cyano-4-formyl-1H-pyrazol-1-yl)piperidine-1-carboxylate (Example 9, Step A) (171 mg, 0.59 mmol) in tetrahydrofuran (2.5 mL) was added. The cold bath was removed, and the reaction mixture was stirred for 3.75 hours at room temperature. The reaction was quenched with saturated aqueous ammonium chloride, and the mixture was extracted twice with ethyl acetate. The combined extracts were washed sequentially with water and brine and then dried over sodium sulfate. The mixture was filtered, and the filtrate was concentrated in vacuo. The residue was purified by silica gel chromatography eluting with a gradient mixture of ethyl acetate in heptane (10 to 100%) to give the title compound as a clear oil (116 mg, 68%). ¹H NMR (500 MHz, deuterochloroform) delta 0.88 (d, J=6.10 Hz, 6H), 1.55-1.67 (m, 2H), 1.68-1.84 (m, 2H), 2.43-2.73 (m, 2H), 3.95 (br. s., 2H), 4.04-4.21 (m, 1H) 4.44-4.67 (m, 1H), 5.02 (d, J=11.22 Hz, 1H), 5.43 (d, J=17.81 Hz, 1H), 6.20 (dd, J=17.81, 11.22 Hz, 1H), 7.27 (s, 1H).

### B) (E,Z)-Isopropyl 4-(5-cyano-4-(2-(2-fluoro-4-(methylsulfonyl)phenyl)prop-1-enyl)-1H-pyrazol-1-yl)piperidine-1-carboxylate

To a solution of isopropyl 4-(5-cyano-4-vinyl-1H-pyrazol-1-yl)piperidine-1-carboxylate (116 mg, 0.4 mmol) and 2-fluoro-4-(methylsulfonyl)-1-(prop-1-en-2-yl)benzene (Preparation 29) (43 mg, 0.20 mmol) in anhydrous dichloromethane (2 mL) was added the second generation Hoveyda-Grubbs catalyst (commercially available from Aldrich) (12.5 mg, 0.020 mmol). The green solution was heated at 40 degrees Celsius for 72 hours periodically adding dichloromethane. The material was concentrated under reduced pressure, and the residue purified by silica gel chromatography (10 to 100% ethyl acetate in heptane) to give the product as an impure oil (8 mg, 8%). This material was used as is. LCMS (APCI): 473.2 (M−1).

### C) Isopropyl 4-(5-cyano-4-{2-[2-fluoro-4-(methylsulfonyl)phenyl]propyl}-1H-pyrazol-1-yl)piperidine-1-carboxylate

A solution of (E,Z)-isopropyl 4-(5-cyano-4-(2-(2-fluoro-4-(methylsulfonyl)-phenyl)prop-1-enyl)-1H-pyrazol-1-yl)piperidine-1-carboxylate (8 mg, 0.02 mmol) in ethyl acetate (3 mL) was hydrogenated on the H-Cube™ at the “full hydrogen” setting using a 10% palladium on carbon cartridge at a flow rate of 1 mL/minute. The material was concentrated in vacuo, and the residue (4 mg) was purified by reversed-phase HPLC (Column: Waters XBridge C18 19×100, 5 micrometer; Mobile phase A: 0.03% ammonium hydroxide in water (v/v); Mobile phase B: 0.03% ammonium hydroxide in acetonitrile (v/v); Gradient: 80% water/20% acetonitrile linear to 0% water/100% acetonitrile in 8.5 minutes, hold at 0% water/100% acetonitrile to 10.0 minutes. Flow: 25 mL/minute) to give the title compound (1.9 mg, 23%): LCMS (ES+): 477.2 (M+1).

### Example 30

### 1-Methylcyclopropyl 4-(5-cyano-4-{[(2-methylpyridin-3-yl)oxy]methyl}-1H-pyrazol-1-yl)piperidine-1-carboxylate

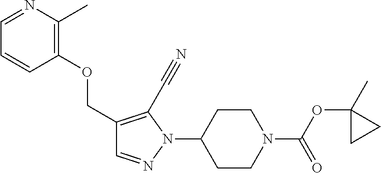

The title compound was prepared using 2-methylpyridin-3-ol, following procedures analogous to Example 13. The crude material was purified by flash chromatography, eluting with a gradient mixture of ethyl acetate in heptane (60 to 100% ethyl acetate) to give 77 mg of the title compound as a white solid. ¹H NMR (400 MHz, deuterochloroform) delta 0.60-0.66 (m, 2H), 0.83-0.90 (m, 2H), 1.55 (s, 3H), 1.96-2.05 (m, 2H), 2.05-2.20 (m, 2H), 2.49 (s, 3H), 2.84-2.98 (m, 2H), 4.11-4.42 (m, 2H), 4.46-4.55 (m, 1H), 5.04 (s, 2H), 7.06-7.16 (m, 2H), 7.65 (s, 1H), 8.12 (dd, J=4.49, 1.56 Hz, 1H).

### Example 31

### 1-Methylcyclopropyl 4-{5-cyano-4-[(2,3,6-trifluorophenoxy)methyl]-1H-pyrazol-1-yl}piperidine-1-carboxylate

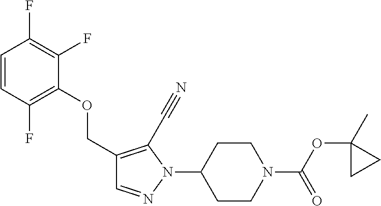

### A) tert-Butyl 4-(5-cyano-4-((2,3,6-trifluorophenoxy)methyl)-1H-pyrazol-1-yl)piperidine-1-carboxylate

tert-Butyl 4-(5-cyano-4-((methylsulfonyloxy)methyl)-1H-pyrazol-1-yl)piperidine-1-carboxylate (Preparation 16) (87.8 mg, 0.228 mmol), 2,3,6-trifluorophenol (51.7 mg, 0.342 mmol), and cesium carbonate (149 mg, 0.456 mmol) were placed in microwave vial and dissolved in acetonitrile (3 mL). The vial was heated in a microwave reactor at 110 degrees Celsius for 20 minutes. The mixture was concentrated under reduced pressure, and the residue was taken up in 1 N sodium hydroxide solution (5 mL) and extracted three times with dichloromethane. The combined organic extracts were washed with brine, dried over sodium sulfate, filtered, and the filtrate was concentrated under reduced pressure. The crude material was purified by chromatography eluting with a 0 to 30% ethyl acetate in heptane gradient to give 36.2 mg of tert-butyl 4-(5-cyano-4-((2,3,6-trifluorophenoxy)methyl)-1H-pyrazol-1-yl)piperidine-1-carboxylate as a clear oil.

### B) 1-Methylcyclopropyl 4-{5-cyano-4-[(2,3,6-trifluorophenoxy)methyl]-1H-pyrazol-1-yl}piperidine-1-carboxylate

1-Methylcyclopropyl 4-{5-cyano-4-[(2,3,6-trifluorophenoxy)methyl]-1H-pyrazol-1-yl}piperidine-1-carboxylate was prepared using commercially available 2,3,6-trifluorophenol, following procedures analogous to Example 13 (B and C). The crude material (17.1 mg) was purified by preparative reverse-phase HPLC on a Sepax 2-Ethyl Pyridine column 250×21.2 mm, 0.005 eluting with a gradient of ethanol in heptane. Analytical LCMS: retention time 11.769 minutes (Phenomenex Luna (2) C₁₈ 150×3.0 mm, 5 micrometer column; 95% water/methanol linear gradient to 100% methanol over 12.5 minutes; 0.1% formic acid modifier; flow rate 0.75 mL/minute; LCMS (ES+): 456.9 (M+Na). ¹H NMR (500 MHz, deuterochloroform) delta 0.64-0.66 (m, 2H), 0.88-0.91 (m, 2H), 1.57 (s, 3H), 2.00 (d, J=10.49 Hz, 2H), 2.07-2.18 (m, 2H), 2.91-2.95 (m, 2H), 4.18 (br. s., 1H), 4.36 (br. s., 1H), 4.50 (tt, J=11.34, 4.15 Hz, 1H), 5.19 (s, 2H), 6.83-6.90 (m, 2H), 7.67 (s, 1H).

### Example 32

### Isopropyl 4-{5-cyano-4-[(2,3,6-trifluorophenoxy)methyl]-1H-pyrazol-1-yl}piperidine-1-carboxylate

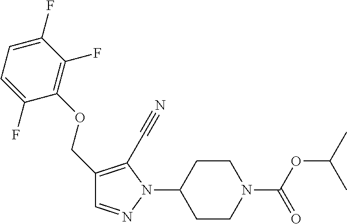

The title compound was prepared using commercially available 2,3,6-trifluorophenol following procedures analogous to Example 11. The crude material was purified by column, chromatography eluting with a 0 to 25% ethyl acetate in heptane gradient to give isopropyl 4-{5-cyano-4-[(2,3,6-trifluorophenoxy)methyl]-1H-pyrazol-1-yl}piperidine-1-carboxylate as a clear oil. ¹H NMR (500 MHz, deuterochloroform) delta 1.26 (d, J=6.10 Hz, 6H), 2.01 (d, J=11.22 Hz, 2H) 2.13 (qd, J=12.28, 4.64 Hz, 2H), 2.88-3.01 (m, 2H), 4.32 (br. s., 2H) 4.51 (tt, J=11.34, 4.15 Hz, 1H), 4.90-4.98 (m,1H), 5.18 (s, 2H), 6.82-6.92 (m, 2H), 7.67 (s, 1H); LCMS (ES+): 423.4 (M+H).

### Example 33

### Isopropyl 4-(5-cyano-4-{[2-fluoro-4-(1-methyl-1H-imidazol-2-yl)phenoxy]methyl}-1H-pyrazol-1-yl)piperidine-1-carboxylate

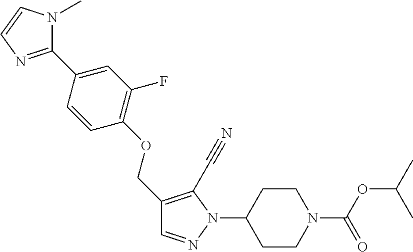

The title compound was prepared from 2-fluoro-4-(1-methyl-1H-imidazol-2-yl)phenol (Preparation 28) and isopropyl 4-(5-cyano-4-((methylsulfonyloxy)methyl)-1H-pyrazol-1-yl)piperidine-1-carboxylate (Preparation 10) following procedures analogous to Example 11. The crude material was purified by preparative reverse-phase HPLC on a Sepax Silica 250×21.2 mm, 0.005 mm, eluting with a gradient of ethanol in heptane. Analytical LCMS: retention time 8.598 minutes (Phenomenex Luna (2) C₁₈ 150×3.0 mm, 5 micrometer column; 95% water/methanol linear gradient to 100% methanol over 12.5 minutes; 0.1% formic acid modifier; flow rate 0.75 mL/minute; LCMS (ES+): 467.0 (M+H). ¹H NMR (500 MHz, deuterochloroform) delta 1.27 (d, J=6.10 Hz, 6H), 1.97-2.09 (m, 2H), 2.16 (m, 2H), 2.93-2.98 (m, 2H), 3.76 (s, 3H) 4.25-4.43 (m, 2H), 4.50-4.57 (m, 1H), 4.91-4.99 (m, 1H), 5.17 (s, 2H), 6.97 (s, 1H), 7.11 (s, 1H), 7.12-7.15 (m, 1H), 7.42 (dd, J=11.71, 1.95 Hz, 1H), 7.38-7.44 (m, 1H), 7.72 (s, 1H).

### Example 34

### Isopropyl 4-(5-cyano-4-{[2-fluoro-4-(1-methyl-1H-imidazol-5-yl)phenoxy]methyl}-1H-pyrazol-1-yl)piperidine-1-carboxylate

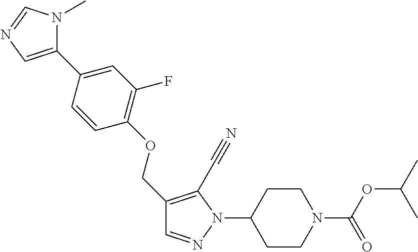

The title compound was prepared from 2-fluoro-4-(1-methyl-1H-imidazol-5-yl)phenol (Preparation 27) and Isopropyl 4-(5-cyano-4-((methylsulfonyloxy)methyl)-1H-pyrazol-1-yl)piperidine-1-carboxylate (Preparation 10) following procedures analogous to Example 11. The crude material was purified by preparative reverse-phase HPLC on a Sepax Silica 250×21.2 mm, 0.005 eluting with a gradient of ethanol in heptane. Analytical LCMS: retention time 8.797 minutes(Phenomenex Luna (2) C₁₈ 150×3.0 mm, 5 micrometer column; 95% water/methanol linear gradient to 100% methanol over 12.5 minutes; 0.1% formic acid modifier; flow rate 0.75 mL/minute; LCMS (ES+): 467.0 (M+H). ¹H NMR (500 MHz, deuterochloroform) delta 1.27 (d, J=6.34 Hz, 6H), 2.03 (d, J=11.22 Hz, 2H), 2.11-2.20 (m, 2H), 2.95 (br. s., 2H), 3.66 (s, 3H), 4.34 (br. s., 2H), 4.50-4.57 (m,1H), 4.94 (dt, J=12.44, 6.22 Hz, 1H), 5.15 (s, 2H), 7.07 (s, 1H), 7.10-7.17 (m, 3H), 7.51 (s, 1H), 7.71 (s, 1H).

### Example 35

### Isopropyl 4-[5-cyano-4-({[2-methyl-6-(1H-1,2,4-triazol-1-yl)pyridin-3-yl]oxy}methyl)-1H-pyrazol-1-yl]piperidine-1-carboxylate

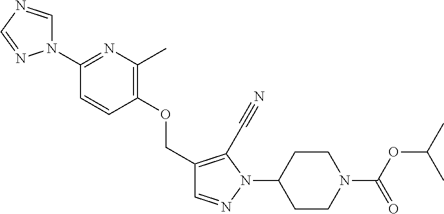

The title compound was prepared using 2-methyl-6-(1H-1,2,4-triazol-1-yl)pyridin-3-ol following procedures analogous to Example 12. The sample was purified by reversed-phase HPLC (Column: Waters XBridge C18 19×100, 5 micrometer; Mobile phase A: 0.03% ammonium hydroxide in water (v/v); Mobile phase B: 0.03% ammonium hyrdroxide in acetonitrile (v/v); Gradient: 80% water/20% acetonitrile linear to 0% water/100% acetonitrile in 8.0 minutes, hold at 0% water/100% acetonitrile to 9.5 minutes. Flow: 25 mL/minute. LCMS (MS ES+: 451.1).

### Example 36

### Isopropyl 4-[5-cyano-4-({[2-methyl-6-(1H-1,2,4-triazol-1-yl)pyridin-3-yl]amino}methyl)-1H-pyrazol-1-yl]piperidine-1-carboxylate

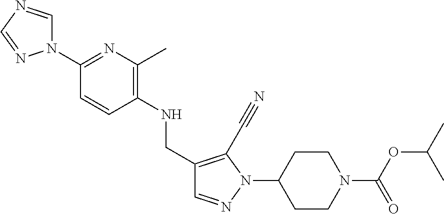

To a stirred solution of isopropyl 4-(5-cyano-4-((methylsulfonyloxy)methyl)-1H-pyrazol-1-yl)piperidine-1-carboxylate (Preparation 10) (44 mg, 0.12 mmol) in 0.75 mL of tetrahydrofuran was added N,N-diisopropylethylamine (0.042 mL, 0.24 mmol) followed by 2-methyl-6-(1H-1,2,4-triazol-1-yl)pyridin-3-amine (21 mg, 0.12 mmol). The reaction mixture was heated at 60 degrees Celsius for 16 hours before it was cooled to room temperature and diluted with water and brine. The mixture was then extracted three times with 15 mL ethyl acetate. The combined organic extracts were washed with brine, dried over sodium sulfate, filtered and the filtrate was concentrated in vacuo to give 52 mg of a yellow foam. The sample was purified by reversed-phase HPLC (Column: Waters Sunfire C18 19×100, 5 micrometer; Mobile phase A: 0.05% trifluoroacetic acid in water (v/v); Mobile phase B: 0.05% trifluoroacetic acid in acetonitrile (v/v)); Gradient: 90% water/10% acetonitrile linear to 0% water/100% acetonitrile in 8.5 minutes, hold at 0% water/100% acetonitrile to 10.0 minutes. Flow: 25 mL/minute. LCMS (MS ES+: 450.1).

### Example 37

### Isopropyl 4-[5-cyano-4-({[2-methyl-6-(methylsulfonyl)pyridin-3-yl]amino}methyl)-1H-pyrazol-1-yl]piperidine-1-carboxylate

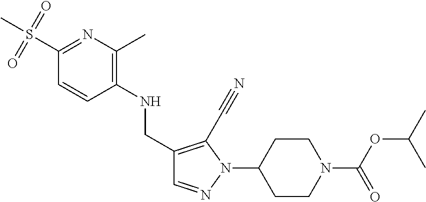

The title compound was prepared using 2-methyl-6-(methylsulfonyl)pyridin-3-amine following procedures analogous to Example 36. The sample was purified by reversed-phase HPLC (Column: Waters XBridge C18 19×100, 5 micrometer; Mobile phase A: 0.03% ammonium hydroxide in water (v/v); Mobile phase B: 0.03% ammonium hyrdroxide in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/minute. LCMS (ES+): 461.0 (M+1).

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having the Formula I: 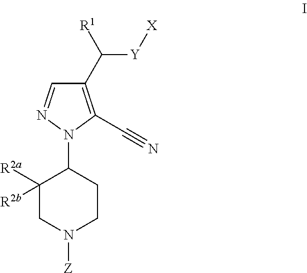
wherein:
X is 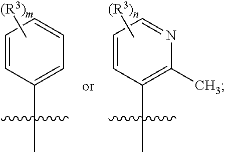
Y is O, CH(R⁵), or NR⁵;
Z is —C(O)—O—R⁶ or pyrimidine substituted with C₁-C₄ alkyl, CF₃, halogen, cyano, C₃-C₆ cycloalkyl or C₃-C₆ cycloalkyl wherein one carbon atom of said cycloalkyl moiety may optionally be substituted with methyl or ethyl;
m is 1, 2, or 3;
n is 0, 1 or 2;
R¹ is hydrogen, C₁-C₄ alkyl, or C₃-C₆ cycloalkyl;
R^{2a} is hydrogen, fluoro or C₁-C₄ alkyl;
R^{2b} is hydrogen or fluoro, with the proviso that when R^{2a} is C₁-C₄ alkyl, R^{2b} is hydrogen;
each R³ is individually selected from the group consisting of: hydroxy, halogen, cyano, C₁-C₄ alkyl, C₁-C₄ alkoxy, C₁-C₄ haloalkyl, C₁-C₄ haloalkoxy, —SO₂—R⁷, —P(O)(OR⁸)(OR⁹), —C(O)—NR⁸R⁹, —N(CH₃)—CO—O—(C₁-C₄)alkyl, —NH—CO—O—(C₁-C₄)alkyl, —NH—CO—(C₁-C₄)alkyl, —N(CH₃)—CO—(C₁-C₄)alkyl, —NH—(CH₂)₂—OH and a 5 to 6-membered heteroaryl group containing 1, 2, 3 or 4 heteroatoms each independently selected from oxygen, nitrogen and sulfur, wherein a carbon atom on said heteroaryl group is optionally substituted with R^{4a} or a nitrogen atom on said heteroaryl group is optionally substituted with R^{4b};
R^{4a} is hydrogen, C₁-C₄ alkyl, C₁-C₄ alkoxy, C₁-C₄ haloalkyl, or halogen, wherein said alkyl is optionally substituted with hydroxyl or C₁-C₄ alkoxy;
R^{4b} is hydrogen, C₁-C₄ alkyl, —CH₂—C₁-C₃ haloalkyl, —C₂-C₄ alkyl-OH or —CH₂—C₁-C₄ alkoxy;
R⁵ is hydrogen or when R¹ is hydrogen then R⁵ is hydrogen or C₁-C₄ alkyl;
R⁶ is C₁-C₄ alkyl or C₃-C₆ cycloalkyl wherein one carbon atom of said cycloalkyl moiety may optionally be substituted with methyl or ethyl;
R⁷ is represented by C₁-C₄ alkyl, C₃-C₆ cycloalkyl, NH₂, or —(CH₂)₂—OH;
R⁸ is represented by hydrogen or C₁-C₄ alkyl; and
R⁹ is represented by hydrogen, C₁-C₄ alkyl, C₃-C₆ cycloalkyl, —(CH₂)₂—OH, —(CH₂)₂—O—CH₃, —(CH₂)₃—OH, —(CH₂)₃—O—CH₃, 3-oxetanyl, or 3-hydroxycyclobutyl;
or when R³ is —C(O)—NR⁸R⁹, R⁸ and R⁹ can be taken together with the nitrogen atom to which they are attached to form an azetidine, a pyrrolidine, a piperidine or a morpholine ring;
or a pharmaceutically acceptable salt thereof.

2. A compound according to claim 1, wherein
X is 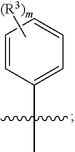
Y is O;
m is 1 or 2;
Z is —C(O)—O—R⁶,
R¹ is hydrogen;
R^{2a} is hydrogen;
R^{2b} is hydrogen; and
each R³ is independently hydroxy, halogen, cyano, CF₃, OCF₃, C₁-C₄ alkyl, C₁-C₄ alkoxy, SO₂—R⁷, —P(O)(OR⁸)(OR⁹), —CO—NR⁸R⁹, or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

3. A compound according to claim 1, wherein
X is 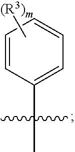
Y is O;
m is 1 or 2;
Z is —C(O)—O—R⁶;
R¹ is hydrogen;
R^{2a} is fluoro;
R^{2b} is hydrogen; and
each R³ is independently hydroxy, halogen, cyano, CF₃, OCF₃, C₁-C₄ alkyl, C₁-C₄ alkoxy, SO₂—R⁷, —P(O)(OR⁸)(OR⁹), —CO—NR⁸R⁹, or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

4. A compound according to claim 1 or 2 wherein each R³ is independently fluoro, methyl, cyano, —C(O)NR⁸R⁹, —SO₂—R⁷, tetrazole, pyrazole, imidazole or triazole.

5. A compound according to claim 1, 2 or 4 wherein
each R³ is independently fluoro, methyl, cyano, —C(O)NR⁸R⁹, —SO₂—R⁷, 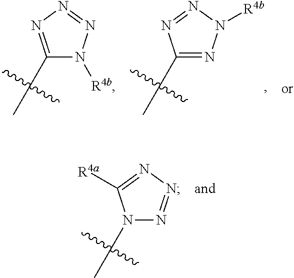
R^{4a} and R^{4b} are each independently hydrogen, C₁-C₄ alkyl, or C₂-C₄ alkyl-OH.

6. A compound according to claim 1 wherein
X is 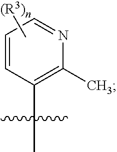
Y is O or NH;
Z is —C(O)—O—R⁸;
n is 0 or 1;
R¹ is hydrogen;
R^{2a} is hydrogen;
R^{2b} is hydrogen; and
R³, if present, is C₁-C₄ alkyl or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

7. A compound according to claim 1 wherein
X is 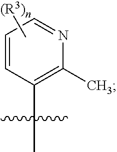
Y is O or NH;
Z is —C(O)—O—R⁶;
n is 0 or 1;
R¹ is hydrogen;
R^{2a} is fluoro;
R^{2b} is hydrogen; and
R³, if present, is C₁-C₄ alkyl or a 5- to 6-membered heteroaryl group containing 1, 2, 3 or 4 heteroatoms each independently selected from oxygen and nitrogen, wherein a carbon atom on said heteroaryl group is optionally substituted with R^{4a} or a nitrogen atom on said heteroaryl group is optionally substituted with R^{4b}.

8. A compound according to any of claims 1-3 wherein R⁶ is isopropyl or 1-methylcyclopropyl.

9. The compound:
1-methylcyclopropyl 4-{4-[(4-carbamoyl-3-fluorophenoxy)methyl]-5-cyano-1H-pyrazol-1-yl}piperidine-1-carboxylate,
1-methylcyclopropyl 4-{4-[(4-carbamoyl-2-fluorophenoxy)methyl]-5-cyano-1H-pyrazol-1-yl}piperidine-1-carboxylate;
isopropyl 4-(5-cyano-4-{[4-(1H-pyrazol-1-yl)phenoxy]methyl}-1H-pyrazol-1-yl)piperidine-1-carboxylate;
1-methylcyclopropyl 4-{5-cyano-4-[(2,3-difluorophenoxy)methyl]-1H-pyrazol-1-yl}piperidine-1-carboxylate;
1-methylcyclopropyl 4-{5-cyano-4-[(2,5-difluorophenoxy)methyl]-1H-pyrazol-1-yl}piperidine-1-carboxylate;
1-methylcyclopropyl 4-{5-cyano-4-[(2,3,6-trifluorophenoxy)methyl]-1H-pyrazol-1-yl}piperidine-1-carboxylate;
isopropyl 4-[5-cyano-4-({2-fluoro-4-[1-(2-hydroxyethyl)-1H-tetrazol-5-yl]phenoxy}methyl)-1H-pyrazol-1-yl]piperidine-1-carboxylate;
isopropyl 4-[5-cyano-4-({2-fluoro-4-[2-(2-hydroxyethyl)-2H-tetrazol-5-yl]phenoxy}methyl)-1H-pyrazol-1-yl]piperidine-1-carboxylate;
isopropyl 4-(5-cyano-4-{[2-fluoro-4-(1-methyl-1H-imidazol-2-yl)phenoxy]methyl}-1H-pyrazol-1-yl)piperidine-1-carboxylate;
1-methylcyclopropyl 4-{5-cyano-4-[(4-cyanophenoxy)methyl]-1H-pyrazol-1-yl}piperidine-1-carboxylate;
1-methylcyclopropyl 4-{4-[(4-carbamoylphenoxy)methyl]-5-cyano-1H-pyrazol-1-yl}piperidine-1-carboxylate,
1-methylcyclopropyl 4-(5-cyano-4-{[4-(1-methyl-1H-tetrazol-5-yl)phenoxy]methyl}-1H-pyrazol-1-yl)piperidine-1-carboxylate;
1-methylcyclopropyl 4-(5-cyano-4-{[2-fluoro-4-(1-methyl-1H-tetrazol-5-yl)phenoxy]methyl}-1H-pyrazol-1-yl)piperidine-1-carboxylate;
isopropyl 4-(5-cyano-4-{[2-fluoro-4-(1-methyl-1H-imidazol-5-yl)phenoxy]methyl}-1H-pyrazol-1-yl)piperidine-1-carboxylate;
isopropyl 4-{5-cyano-4-[(2,3,6-trifluorophenoxy)methyl]-1H-pyrazol-1-yl}piperidine-1-carboxylate;
isopropyl 4-{5-cyano-4-[(2,4-difluorophenoxy)methyl]-1H-pyrazol-1-yl}piperidine-1-carboxylate;
1-methylcyclopropyl 4-(5-cyano-4-{[(2-methylpyridin-3-yl)oxy]methyl}-1H-pyrazol-1-yl)piperidine-1-carboxylate;
isopropyl 4-[5-cyano-4-({[2-methyl-6-(1H-1,2,4-triazol-1-yl)pyridin-3-yl]oxy}methyl)-1H-pyrazol-1-yl]piperidine-1-carboxylate;
isopropyl 4-[5-cyano-4-({[2-methyl-6-(1H-1,2,4-triazol-1-yl)pyridin-3-yl]amino}methyl)-1H-pyrazol-1-yl]piperidine-1-carboxylate;
isopropyl 4-[5-cyano-4-({[2-methyl-6-(methylsulfonyl)pyridin-3-yl]amino}methyl)-1H-pyrazol-1-yl]piperidine-1-carboxylate;
isopropyl 4-{5-cyano-4-[(2-methylphenoxy)methyl]-1H-pyrazol-1-yl}piperidine-1-carboxylate;
isopropyl 4-(5-cyano-4-{[2-fluoro-4-(1-methyl-1H-tetrazol-5-yl)phenoxy]methyl}-1H-pyrazol-1-yl)piperidine-1-carboxylate;
isopropyl 4-(5-cyano-4-{[2-fluoro-4-(2-methyl-2H-tetrazol-5-yl)phenoxy]methyl}-1H-pyrazol-1-yl)piperidine-1-carboxylate;
isopropyl 4-(5-cyano-4-{[(2-methylpyridin-3-yl)amino]methyl}-1H-pyrazol-1-yl)piperidine-1-carboxylate;
isopropyl 4-(5-cyano-4-{1-[(2-methylpyridin-3-yl)oxy]ethyl}-1H-pyrazol-1-yl)piperidine-1-carboxylate;
isopropyl 4-[5-cyano-4-({[2-fluoro-4-(methylsulfonyl)phenyl]amino}methyl)-1H-pyrazol-1-yl]piperidine-1-carboxylate;
isopropyl 4-(5-cyano-4-{1-[2-fluoro-4-(methylsulfonyl)phenoxy]ethyl}-1H-pyrazol-1-yl)piperidine-1-carboxylate;
isopropyl 4-(5-cyano-4-{2-[2-fluoro-4-(methylsulfonyl)phenyl]propyl}-1H-pyrazol-1-yl)piperidine-1-carboxylate;
isopropyl 4-(5-cyano-4-{[2-fluoro-4-(1H-tetrazol-5-yl)phenoxy]methyl}-1H-pyrazol-1-yl)piperidine-1-carboxylate;
isopropyl 4-(5-cyano-4-{2-[2-fluoro-4-(methylsulfonyl)phenyl]ethyl}-1H-pyrazol-1-yl)piperidine-1-carboxylate;
isopropyl 4-{5-cyano-4-[(4-cyano-2-fluorophenoxy)methyl]-1H-pyrazol-1-yl}piperidine-1-carboxylate;
isopropyl 4-(5-cyano-4-{[4-(dimethoxyphosphoryl)-2-fluorophenoxy]methyl}-1H-pyrazol-1-yl)piperidine-1-carboxylate;
isopropyl 4-(5-cyano-4-{[(2-methylpyridin-3-yl)oxy]methyl}-1H-pyrazol-1-yl)piperidine-1-carboxylate;
isopropyl 4-[5-cyano-4-({2-fluoro-4-[(2-hydroxyethyl)sulfonyl]phenoxy}methyl)-1H-pyrazol-1-yl]piperidine-1-carboxylate;
isopropyl 4-(5-cyano-4-{[2-fluoro-4-(1H-tetrazol-1-yl)phenoxy]methyl}-1H-pyrazol-1-yl)piperidine-1-carboxylate;
isopropyl 4-(5-cyano-4-{[4-(1H-tetrazol-1-yl)phenoxy]methyl}-1H-pyrazol-1-yl)piperidine-1-carboxylate; or
isopropyl 4-(5-cyano-4-{[2-fluoro-4-(methylsulfonyl)phenoxy]methyl}-1H-pyrazol-1-yl)piperidine-1-carboxylate;
or a pharmaceutically acceptable salt thereof.

10. A pharmaceutical composition comprising a compound according to any of claim 1 or 9, present in a therapeutically effective amount, in admixture with at least one pharmaceutically acceptable excipient.

11. The composition of claim 10 further comprising at least one additional pharmaceutical agent selected from the group consisting of an anti-obesity agent and an anti-diabetic agent.

12. The composition of claim 11 wherein said anti-obesity agent is selected from the group consisting of dirlotapide, mitratapide, implitapide, R56918 (CAS No. 403987), CAS No. 913541-47-6, lorcaserin, cetilistat, PYY_{3-36,} naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

13. The composition of claim 11 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

14. A method for the treatment of diabetes comprising the administration of an effective amount of compound according to any of claim 1 or 9 to a patient in need thereof.

15. A method for treating a metabolic or metabolic-related disease, condition or disorder comprising the step of administering to a patient a therapeutically effective amount of a compound of any one of claim 1 or 9.

16. A method for treating a condition selected from the group consisting of hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, comprising the administration of an effective amount of a compound according to any of claim 1 or 9.

17. A method for treating a metabolic or metabolic-related disease, condition or disorder comprising the step of administering to a patient in need of such treatment two separate pharmaceutical compositions comprising
(i) a first composition according to claim 12; and
(ii) a second composition comprising at least one additional pharmaceutical agent selected from the group consisting of an anti-obesity agent and an anti-diabetic agent, and at least one pharmaceutically acceptable excipient.

18. The method of claim 17 wherein said first composition and said second composition are administered simultaneously.

19. The method of claim 17 wherein said first composition and said second composition are administered sequentially and in any order.

20. The use of a compound of claim 1 or 9 in the manufacture of a medicament for treating a disease, condition or disorder that modulates the activity of G-protein-coupled receptor GPR119.

21. The use of a compound according to any of claim 1 or 9 in the preparation of a medicament for the treatment of diabetes or a morbidity associated with said diabetes.
